(19) United States
(12) Patent Application Publication
(10) Pub. No.: US 2007/0155739 A1
(43) Pub. Date: Jul. 5, 2007

(54) Substituted bis-amide metalloprotease inhibitors

(75) Inventors: Irving Sucholeiki, Winchester, MA (US); Timothy Powers, Boston, MA (US); Christian Gege, Ehingen (DE); Harald Bluhm, Dossenheim (DE); Rory Dodd, Watertown, MA (US); Hongbo Deng, Southborough, MA (US); Xinyuan Wu, Newton, MA (US); Christoph Steeneck, Dossenheim (DE)

Correspondence Address: HOFFMANN & BARON, LLP, 6900 JERICHO TURNPIKE, SYOSSET, NY, 11791, US

(73) Assignee: ALANTOS PHARMACEUTICALS, INC.

(21) Appl. No.: 11/646,650

(22) Filed: Dec. 28, 2006

Related U.S. Application Data
(60) Provisional application No. 60/755,539, filed on Dec. 30, 2005.

(51) Int. Cl. A61K 31/5383; A61K 31/5377; A61K 31/506; C07D 491/02; C07D 413/02; C07D 403/02

(52) U.S. Cl. 514/230.5; 514/235.5; 514/252.14; 514/256; 544/105; 544/122; 544/295; 544/333; 544/335; 544/296

(57) ABSTRACT

This invention relates to substituted bis-amide pyrimidine compounds of Formula (I), which are useful for the treatment of metalloprotease mediated diseases, in particular MMP-13 related diseases.

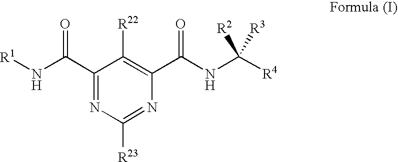

### CROSS REFERENCE TO RELATED APPLICATIONS

This application is a continuation in part of U.S. Application No. 60/755,539, filed Dec. 30, 2005, the contents of which are hereby incorporated by reference.

### FIELD OF THE INVENTION

The present invention relates generally to bis-amide containing metalloprotease inhibiting compounds, and more particularly to substituted bis-amide MMP-13 inhibiting compounds.

### BACKGROUND OF THE INVENTION

Matrix metalloproteinases (MMPs) and aggrecanases (ADAMTS=a disintegrin and metalloproteinase with thrombospondin motif) are a family of structurally related zinc-containing enzymes that have been reported to mediate the breakdown of connective tissue in normal physiological processes such as embryonic development, reproduction, and tissue remodelling. Over-expression of MMPs and aggrecanases or an imbalance between extracellular matrix synthesis and degradation has been suggested as factors in inflammatory, malignant and degenerative disease processes. MMPs and aggrecanases are, therefore, targets for therapeutic inhibitors in several inflammatory, malignant and degenerative diseases such as rheumatoid arthritis, osteoarthritis, osteoporosis, periodontitis, multiple sclerosis, gingivitis, corneal epidermal and gastric ulceration, atherosclerosis, neointimal proliferation (which leads to restenosis and ischemic heart failure) and tumor metastasis.

The ADAMTSs are a group of proteases that are encoded in 19 ADAMTS genes in humans. The ADAMTSs are extracellular, multidomain enzymes whose functions include collagen processing, cleavage of the matrix proteoglycans, inhibition of angiogenesis and blood coagulation homoeostasis (Biochem. J. 2005, 386, 15-27; Arthritis Res. Ther. 2005, 7, 160-169; Curr. Med. Chem. Anti-Inflammatory Anti-Allergy Agents 2005, 4, 251-264).

The mammalian MMP family has been reported to include at least 20 enzymes, (Chem. Rev. 1999, 99, 2735-2776). Collagenase-3 (MMP-13) is among three collagenases that have been identified. Based on identification of domain structures for individual members of the MMP family, it has been determined that the catalytic domain of the MMPs contains two zinc atoms; one of these zinc atoms performs a catalytic function and is coordinated with three histidines contained within the conserved amino acid sequence of the catalytic domain. MMP-13 is over-expressed in rheumatoid arthritis, osteoarthritis, abdominal aortic aneurysm, breast carcinoma, squamous cell carcinomas of the head and neck, and vulvar squamous cell carcinoma. The principal substrates of MMP-13 are fibrillar collagens (types I, I, III) and gelatins, proteoglycans, cytokines and other components of ECM (extracellular matrix).

The activation of the MMPs involves the removal of a propeptide, which features an unpaired cysteine residue complexes the catalytic zinc (II) ion. X-ray crystal structures of the complex between MMP-3 catalytic domain and TIMP-1 and MMP-14 catalytic domain and TIMP-2 also reveal ligation of the catalytic zinc (II) ion by the thiol of a cysteine residue. The difficulty in developing effective MMP inhibiting compounds comprises several factors, including choice of selective versus broad-spectrum MMP inhibitors and rendering such compounds bioavailable via an oral route of administration.

MMP-3 (stromelysin-1; transin-1) is another member of the MMP family (Woesner; FASEB J. 1991; 5:2145-2154). Human MMP-3 was initially isolated from cultured human synoviocytes. It is also expressed by chondrocytes and has been localized in OA cartilage and synovial tissues (Case; Am. J. Pathol. 1989 December; 135(6):1055-64).

MMP-3 is produced by basal keratinocytes in a variety of chronic ulcers. MMP-3 mRNA and Protein were detected in basal keratinocytes adjacent to but distal from the wound edge in what probably represents the sites of proliferating epidermis. MMP-3 may this prevent the epidermis from healing (Saarialho-Kere, J. Clin. Invest. 1994 July; 94(1):79-88)).

MMP-3 serum protein levels are significantly elevated in patients with early and long-term rheumatoid arthritis (Yamanaka; Arthritis Rheum. 2000 April; 43(4):852-8) and in osteoarthritis patients (Bramono; Clin Orthop Relat Res. 2004 November; (428):272-85) as well as in other inflammatory diseases like systemic lupus erythematosis and ankylosing spondylitis (Chen, Rheumatology 2006 April; 45(4):414-20.).

MMP-3 acts on components of the ECM as aggrecan, fibronectin, gelatine, laminin, elastin, fibrillin and others and on collagens of type III, IV, V, VI, KX, X (Bramono; Clin Orthop Relat Res. 2004 November; (428):272-85). On collagens of type II and IX, MMP-3 exhibits telopeptidase activity (Sandell, Arthritis Res. 2001; 3(2):107-13; Eyre, Clin Orthop Relat Res. 2004 October; (427 Suppl):S118-22.). MMP-3 can activate other MMP family members as MMP-1; MMP-7; MMP-8; MMP-9 and MMP-13 (Close, Ann Rheum Dis 2001 November; 60 Suppl 3:iii62-7).

MMP-3 is involved in the regulation of cytokines and chemokines by releasing TGFβ1 from the ECM, activating TNFα, inactivation of IL-1β and release of IGF (Parks, Nat Rev Immunol. 2004 August; 4(8):617-29). A potential role for MMP-3 in the regulation of macrophate infiltration is based on the ability of the enzyme to converse active MCP species into antagonistic peptides (McQuibban, Blood. 2002 Aug. 15; 100(4):1160-7.).

A series of MMP-13 inhibiting compounds containing a bis-amide functional group in combination with a pyrimidine ring is disclosed in WO 02/064571, WO 04/041788 and WO 04/060883. This invention discloses metalloprotease inhibitors with surprising and unexpected improvements in the properties metalloprotease inhibitors bearing an R² substituent in the compounds of Claim 1. Furthermore, the specific substitution (R² vs. R³) is critical as compounds bearing an R³ substituent have poorer activity. The unexpected advantages observed for selective R²-substituted compounds of this invention include improvements in microsomal stability and cell viability, as is evident by comparing the results observed for the unsubstituted pyrimidine-4,6-dicarboxylic acid 4-(3-methoxybenzylamide) 6-[4-(1H-tetrazol-5-yl)-benzylamide] (Example 1040d) with the improvements seen with Example 1005. It is believed that these new findings and the specific structural modifications which this invention discloses will lead to inhibitors of metalloproteases, in particular MMP-13 with improved pharmaceutical value.

### SUMMARY OF THE INVENTION

The present invention relates to a new class of substituted bis-amide containing pharmaceutical agents. In particular, the present invention provides a new class of metalloprotease inhibiting compounds containing a pyrimidinyl bis-amide group in combination with a substituted moiety that exhibit potent MMP-13 inhibiting activity and are highly selective toward MMP-13 compared to currently known MMP inhibitors.

The present invention provides a new class of substituted bis-amide metalloprotease inhibiting compounds that are represented by the general Formula (I):

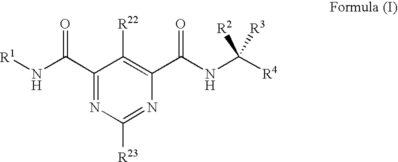

Wherein R¹, R², R³, R⁴, R²², and R²³ are as described hereinbelow.

The substituted bis-amide metalloprotease inhibiting compounds of the present invention may be used in the treatment of metalloprotease mediated diseases.

In particular, the substituted bis-amide metalloprotease inhibiting compounds of the present invention may be used in the treatment of MMP-13 mediated osteoarthritis and may be used for other MMP-13 mediated symptoms, inflammatory, malignant and degenerative diseases characterized by excessive extracellular matrix degradation and/or remodelling, such as cancer, and chronic inflammatory diseases such as arthritis, rheumatoid arthritis, osteoarthritis atherosclerosis, abdominal aortic aneurysm, inflammation, multiple sclerosis, and chronic obstructive pulmonary disease, and pain, such as inflammatory pain, bone pain and joint pain.

The present invention also provides substituted bis-amide metalloprotease inhibiting compounds that are useful as active ingredients in pharmaceutical compositions for treatment or prevention of metalloprotease—especially MMP-13—mediated diseases. The present invention also contemplates use of such compounds in pharmaceutical compositions for oral or parenteral administration, comprising one or more of the substituted bis-amide metalloprotease inhibiting compounds disclosed herein.

The present invention further provides methods of inhibiting metalloproteases, by administering formulations, including, but not limited to, oral, rectal, topical, intravenous, parenteral (including, but not limited to, intramuscular, intravenous), ocular (ophthalmic), transdermal, inhalative (including, but not limited to, pulmonary, aerosol inhalation), nasal, sublingual, subcutaneous or intraarticular formulations, comprising the heterobicyclic metalloprotease inhibiting compounds by standard methods known in medical practice, for the treatment of diseases or symptoms arising from or associated with metalloprotease, especially MMP-13, including prophylactic and therapeutic treatment. Although the most suitable route in any given case will depend on the nature and severity of the conditions being treated and on the nature of the active ingredient. The compounds from this invention are conveniently presented in unit dosage form and prepared by any of the methods well-known in the art of pharmacy.

The substituted bis-amide metalloprotease inhibiting compounds of the present invention may be used in combination with a disease modifying antirheumatic drug, a nonsteroidal anti-inflammatory drug, a COX-2 selective inhibitor, a COX-1 inhibitor, an immunosuppressive, a steroid, a biological response modifier or other anti-inflammatory agents or therapeutics useful for the treatment of chemokine mediated diseases.

### DETAILED DESCRIPTION OF THE INVENTION

The terms “alkyl” or “alk”, as used herein alone or as part of another group, denote optionally substituted, straight and branched chain saturated hydrocarbon groups, preferably having 1 to 10 carbons in the normal chain, most preferably lower alkyl groups. Exemplary unsubstituted such groups include methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl and the like. Exemplary substituents may include, but are not limited to, one or more of the following groups: halo, alkoxy, alkylthio, alkenyl, alkynyl, aryl (e.g., to form a benzyl group), cycloalkyl, cycloalkenyl, hydroxy or protected hydroxy, carboxyl (—COOH), alkyloxycarbonyl, alkylcarbonyloxy, alkylcarbonyl, carbamoyl (NH₂—CO—), substituted carbamoyl ((R¹⁰)(R¹¹)N—CO— wherein R¹⁰ or R¹¹ are as defined below, except that at least one of R¹⁰ or R¹¹ is not hydrogen), amino, heterocyclo, mono- or dialkylamino, or thiol (—SH).

The terms “lower alk” or “lower alkyl” as used herein, denote such optionally substituted groups as described above for alkyl having 1 to 4 carbon atoms in the normal chain.

The term “alkoxy” denotes an alkyl group as described above bonded through an oxygen linkage (—O—).

The term “alkenyl”, as used herein alone or as part of another group, denotes optionally substituted, straight and branched chain hydrocarbon groups containing at least one carbon to carbon double bond in the chain, and preferably having 2 to 10 carbons in the normal chain. Exemplary unsubstituted such groups include ethenyl, propenyl, isobutenyl, butenyl, pentenyl, hexenyl, heptenyl, octenyl, nonenyl, decenyl, and the like. Exemplary substituents may include, but are not limited to, one or more of the following groups: halo, alkoxy, alkylthio, alkyl, alkynyl, aryl, cycloalkyl, cycloalkenyl, hydroxy or protected hydroxy, carboxyl (—COOH), alkyloxycarbonyl, alkylcarbonyloxy, alkylcarbonyl, carbamoyl (NH₂—CO—), substituted carbamoyl ((R¹⁰)(R¹¹)N—CO— wherein R¹⁰ or R¹¹ are as defined below, except that at least one of R¹⁰ or R¹¹ is not hydrogen), amino, heterocyclo, mono- or dialkylamino, or thiol (—SH).

The term “alkynyl”, as used herein alone or as part of another group, denotes optionally substituted, straight and branched chain hydrocarbon groups containing at least one carbon to carbon triple bond in the chain, and preferably having 2 to 10 carbons in the normal chain. Exemplary unsubstituted such groups include, but are not limited to, ethynyl, propynyl, butynyl, pentynyl, hexynyl, heptynyl, octynyl, nonynyl, decynyl, and the like. Exemplary substituents may include, but are not limited to, one or more of the following groups: halo, alkoxy, alkylthio, alkyl, alkenyl, aryl, cycloalkyl, cycloalkenyl, hydroxy or protected hydroxy, carboxyl (—COOH), alkyloxycarbonyl, alkylcarbonyloxy, alkylcarbonyl, carbamoyl (NH₂—CO—), substituted carbamoyl ((R¹⁰(R¹¹)N—CO— wherein R¹⁰ or R¹¹ are as defined below, except that at least one of R¹⁰ or R¹¹ is not hydrogen), amino, heterocyclo, mono- or dialkylamino, or thiol (—SH).

The term “cycloalkyl”, as used herein alone or as part of another group, denotes optionally substituted, saturated cyclic hydrocarbon ring systems, including bridged ring systems, desirably containing 1 to 3 rings and 3 to 9 carbons per ring. Exemplary unsubstituted such groups include, but are not limited to, cyclopropyl, cyclobutyl, cyclopentyl, cyclohexyl, cycloheptyl, cyclooctyl, cyclodecyl, cyclododecyl, and adamantyl. Exemplary substituents include, but are not limited to, one or more alkyl groups as described above, or one or more groups described above as alkyl substituents.

The term “bicycloalkyl”, as used herein alone or as part of another group, denotes optionally substituted, saturated cyclic bridged hydrocarbon ring systems, desirably containing 2 or 3 rings and 3 to 9 carbons per ring. Exemplary unsubstituted such groups include, but are not limited to, adamantyl, bicyclo[2.2.2]octane, bicyclo[2.2.1]heptane and cubane. Exemplary substituents include, but are not limited to, one or more alkyl groups as described above, or one or more groups described above as alkyl substituents.

The term “spiroalkyl”, as used herein alone or as part of another group, denotes optionally substituted, saturated hydrocarbon ring systems, wherein two rings of 3 to 9 carbons per ring are bridged via one carbon atom. Exemplary unsubstituted such groups include, but are not limited to, spiro[3.5]nonane, spiro[4.5]decane or spiro[2.5]octane. Exemplary substituents include, but are not limited to, one or more alkyl groups as described above, or one or more groups described above as alkyl substituents.

The term “spiroheteroalkyl”, as used herein alone or as part of another group, denotes optionally substituted, saturated hydrocarbon ring systems, wherein two rings of 3 to 9 carbons per ring are bridged via one carbon atom and at least one carbon atom is replaced by a heteroatom independently selected from N, O and S. The nitrogen and sulfur heteroatoms may optionally be oxidized. Exemplary unsubstituted such groups include, but are not limited to, 1,3-diaza-spiro[4.5]decane-2,4-dione. Exemplary substituents include, but are not limited to, one or more alkyl groups as described above, or one or more groups described above as alkyl substituents.

The terms “ar” or “aryl”, as used herein alone or as part of another group, denote optionally substituted, homocyclic aromatic groups, preferably containing 1 or 2 rings and 6 to 12 ring carbons. Exemplary unsubstituted such groups include, but are not limited to, phenyl, biphenyl, and naphthyl. Exemplary substituents include, but are not limited to, one or more nitro groups, alkyl groups as described above or groups described above as alkyl substituents.

The term “heterocycle” or “heterocyclic system” denotes a heterocyclyl, heterocyclenyl, or heteroaryl group as described herein, which contains carbon atoms and from 1 to 4 heteroatoms independently selected from the group consisting of N, O and S and including any bicyclic or tricyclic group in which any of the above-defined heterocyclic rings is fused to one or more heterocycle, aryl or cycloalkyl groups. The nitrogen and sulfur heteroatoms may optionally be oxidized. The heterocyclic ring may be attached to its pendant group at any heteroatom or carbon atom which results in a stable structure. The heterocyclic rings described herein may be substituted on carbon or on a nitrogen atom.

Examples of heterocycles include, but are not limited to, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolinyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isatinoyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, oxindolyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl.

Further examples of heterocycles include, but not are not limited to, “heterobicycloalkyl” groups such as 7-oxa-bicyclo[2.2.1]heptane, 7-aza-bicyclo[2.2.1]heptane, and 1-aza-bicyclo[2.2.2]octane.

“Heterocyclenyl” denotes a non-aromatic monocyclic or multicyclic hydrocarbon ring system of about 3 to about 10 atoms, desirably about 4 to about 8 atoms, in which one or more of the carbon atoms in the ring system is/are hetero element(s) other than carbon, for example nitrogen, oxygen or sulfur atoms, and which contains at least one carbon-carbon double bond or carbon-nitrogen double bond. Ring sizes of rings of the ring system may include 5 to 6 ring atoms. The designation of the aza, oxa or thia as a prefix before heterocyclenyl define that at least a nitrogen, oxygen or sulfur atom is present respectively as a ring atom. The heterocyclenyl may be optionally substituted by one or more substituents as defined herein. The nitrogen or sulphur atom of the heterocyclenyl may also be optionally oxidized to the corresponding N-oxide, S-oxide or S,S-dioxide. “Heterocyclenyl” as used herein includes by way of example and not limitation those described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and “J. Am. Chem. Soc.”, 82:5566 (1960), the contents all of which are incorporated by reference herein. Exemplary monocyclic azaheterocyclenyl groups include, but are not limited to, 1,2,3,4-tetrahydrohydropyridine, 1,2-dihydropyridyl, 1,4-dihydropyridyl, 1,2,3,6-tetrahydropyridine, 1,4,5,6-tetrahydropyrimidine, 2-pyrrolinyl, 3-pyrrolinyl, 2-imidazolinyl, 2-pyrazolinyl, and the like. Exemplary oxaheterocyclenyl groups include, but are not limited to, 3,4-dihydro-2H-pyran, dihydrofuranyl, and fluorodihydrofuranyl. An exemplary multicyclic oxaheterocyclenyl group is 7-oxabicyclo[2.2.1]heptenyl.

“Heterocyclyl,” or “heterocycloalkyl,” denotes a non-aromatic saturated monocyclic or multicyclic ring system of about 3 to about 10 carbon atoms, desirably 4 to 8 carbon atoms, in which one or more of the carbon atoms in the ring system is/are hetero element(s) other than carbon, for example nitrogen, oxygen or sulfur. Ring sizes of rings of the ring system may include 5 to 6 ring atoms. The designation of the aza, oxa or thia as a prefix before heterocyclyl define that at least a nitrogen, oxygen or sulfur atom is present respectively as a ring atom. The heterocyclyl may be optionally substituted by one or more substituents which may be the same or different, and are as defined herein. The nitrogen or sulphur atom of the heterocyclyl may also be optionally oxidized to the corresponding N-oxide, S-oxide or S,S-dioxide.

“Heterocyclyl” as used herein includes by way of example and not limitation those described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and “J. Am. Chem. Soc.”, 82:5566 (1960). Exemplary monocyclic heterocyclyl rings include, but are not limited to, piperidyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like.

“Heteroaryl” denotes an aromatic monocyclic or multicyclic ring system of about 5 to about 10 atoms, in which one or more of the atoms in the ring system is/are hetero element(s) other than carbon, for example nitrogen, oxygen or sulfur. Ring sizes of rings of the ring system include 5 to 6 ring atoms. The “heteroaryl” may also be substituted by one or more subsituents which may be the same or different, and are as defined herein. The designation of the aza, oxa or thia as a prefix before heteroaryl define that at least a nitrogen, oxygen or sulfur atom is present respectively as a ring atom. A nitrogen atom of a heteroaryl may be optionally oxidized to the corresponding N-oxide. Heteroaryl as used herein includes by way of example and not limitation those described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and “J. Am. Chem. Soc.”, 82:5566 (1960). Exemplary heteroaryl and substituted heteroaryl groups include, but are not limited to, pyrazinyl, thienyl, isothiazolyl, oxazolyl, pyrazolyl, furazanyl, pyrrolyl, 1,2,4-thiadiazolyl, pyridazinyl, quinoxalinyl, phthalazinyl, imidazo[1,2-a]pyridine, imidazo[2,1-b]thiazolyl, benzofurazanyl, azaindolyl, benzimidazolyl, benzothienyl, thienopyridyl, thienopyrimidyl, pyrrolopyridyl, imidazopyridyl, benzoazaindole, 1,2,3-triazinyl, 1,2,4-triazinyl, 1,3,5-triazinyl, benzthiazolyl, dioxolyl, furanyl, imidazolyl, indolyl, indolizinyl, isoxazolyl, isoquinolinyl, isothiazolyl, morpholino, oxadiazolyl, oxazinyl, oxiranyl, piperazinyl, piperidinyl, pyranyl, pyrazinyl, pyridazinyl, pyrazolyl, pyridyl, pyrimidinyl, pyrrolyl, pyrrolidinyl, quinazolinyl, quinolinyl, tetrazinyl, tetrazolyl, 1,3,4-thiadiazolyl, 1,2,3-thiadiazolyl, 1,2,4-thiadiazolyl, 1,2,5-thiadiazolyl, thiatriazolyl, thiazinyl, thiazolyl, thienyl, 5-thioxo-1,2,4-diazolyl, thiomorpholino, thiophenyl, thiopyranyl, triazolyl and triazolonyl.

The phrase “fused” means, that the group, mentioned before “fused” is connected via two adjacent atoms to the ring system mentioned after “fused” to form a bicyclic system. For example, “heterocycloalkyl fused aryl” includes, but is not limited to, 2,3-dihydro-benzo[1,4]dioxine, 4H-benzo[1,4]oxazin-3-one, 3H-Benzooxazol-2-one and 3,4-dihydro-2H-benzo[f][1,4]oxazepin-5-one.

The term “amino” denotes the radical —NH₂ wherein one or both of the hydrogen atoms may be replaced by an optionally substituted hydrocarbon group. Exemplary amino groups include, but are not limited to, n-butylamino, tert-butylamino, methylpropylamino and ethyldimethylamino.

The term “cycloalkylalkyl” denotes a cycloalkyl-alkyl group wherein a cycloalkyl as described above is bonded through an alkyl, as defined above. Cycloalkylalkyl groups may contain a lower alkyl moiety. Exemplary cycloalkylalkyl groups include, but are not limited to, cyclopropylmethyl, cyclopentylmethyl, cyclohexylmethyl, cyclopropylethyl, cyclopentylethyl, cyclohexylpropyl, cyclopropylpropyl, cyclopentylpropyl, and cyclohexylpropyl.

The term “arylalkyl” denotes an aryl group as described above bonded through an alkyl, as defined above.

The term “heteroarylalkyl” denotes a heteroaryl group as described above bonded through an alkyl, as defined above.

The term “heterocyclylalkyl,” or “heterocycloalkylalkyl,” denotes a heterocyclyl group as described above bonded through an alkyl, as defined above.

The terms “halogen”, “halo”, or “hal”, as used herein alone or as part of another group, denote chlorine, bromine, fluorine, and iodine.

The term “haloalkyl” denotes a halo group as described above bonded though an alkyl, as defined above. Fluoroalkyl is an exemplary group.

The term “aminoalkyl” denotes an amino group as defined above bonded through an alkyl, as defined above.

The term “pharmaceutically acceptable salts” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. Examples therefore may be, but are not limited to, sodium, potassium, choline, lysine, arginine or N-methyl-glucamine salts, and the like.

The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as, but not limited to, hydrochloric, hydrobromic, sulfuric, sulfamic, phosphoric, nitric and the like; and the salts prepared from organic acids such as, but not limited to, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Organic solvents include, but are not limited to, nonaqueous media like ethers, ethyl acetate, ethanol, isopropanol, or acetonitrile. Lists of suitable salts are found in Remington's Pharmaceutical Sciences, 18th ed., Mack Publishing Company, Easton, Pa., 1990, p. 1445, the disclosure of which is hereby incorporated by reference.

The phrase “pharmaceutically acceptable” denotes those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The phrase “pharmaceutically acceptable carrier” denotes media generally accepted in the art for the delivery of biologically active agents to mammals, e.g., humans. Such carriers are generally formulated according to a number of factors well within the purview of those of ordinary skill in the art to determine and account for. These include, without limitation: the type and nature of the active agent being formulated; the subject to which the agent-containing composition is to be administered; the intended route of administration of the composition; and, the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, well known to those of ordinary skill in the art. Non-limiting examples of a pharmaceutically acceptable carrier are hyaluronic acid and salts thereof, and microspheres (including, but not limited to poly(D,L)-lactide-co-glycolic acid copolymer (PLGA), poly(L-lactic acid) (PLA), poly(caprolactone (PCL) and bovine serum albumin (BSA)). Descriptions of suitable pharmaceutically acceptable carriers, and factors involved in their selection, are found in a variety of readily available sources, e.g., Remington's Pharmaceutical Sciences, 17th ed., Mack Publishing Company, Easton, Pa., 1985, the contents of which are incorporated herein by reference.

Pharmaceutically acceptable carriers particularly suitable for use in conjunction with tablets include, for example, inert diluents, such as celluloses, calcium or sodium carbonate, lactose, calcium or sodium phosphate; disintegrating agents, such as croscarmellose sodium, cross-linked povidone, maize starch, or alginic acid; binding agents, such as povidone, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

The compositions of the invention may also be formulated as suspensions including a compound of the present invention in admixture with at least one pharmaceutically acceptable excipient suitable for the manufacture of a suspension. In yet another embodiment, pharmaceutical compositions of the invention may be formulated as dispersible powders and granules suitable for preparation of a suspension by the addition of suitable excipients.

Carriers suitable for use in connection with suspensions include suspending agents, such as sodium carboxymethylcellulose, methylcellulose, hydroxypropyl methylcelluose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); and thickening agents, such as carbomer, beeswax, hard paraffin or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

Cyclodextrins may be added as aqueous solubility enhancers. Preferred cyclodextrins include hydroxypropyl, hydroxyethyl, glucosyl, maltosyl and maltotriosyl derivatives of α-, β, and γ-cyclodextrin. The amount of solubility enhancer employed will depend on the amount of the compound of the present invention in the composition.

The term “formulation” denotes a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical formulations of the present invention encompass any composition made by admixing a compound of the present invention and a pharmaceutical carrier.

The term “N-oxide” denotes compounds that can be obtained in a known manner by reacting a compound of the present invention including a nitrogen atom (such as in a pyridyl group) with hydrogen peroxide or a peracid, such as 3-chloroperoxy-benzoic acid, in an inert solvent, such as dichloromethane, at a temperature between about −10-80° C., desirably about 0° C.

The term “polymorph” denotes a form of a chemical compound in a particular crystalline arrangement. Certain polymorphs may exhibit enhanced thermodynamic stability and may be more suitable than other polymorphic forms for inclusion in pharmaceutical formulations.

The compounds of the invention can contain one or more chiral centers and/or double bonds and, therefore, exist as stereoisomers, such as double-bond isomers (i.e., geometric isomers), enantiomers, or diastereomers. According to the invention, the chemical structures depicted herein, and therefore the compounds of the invention, encompass all of the corresponding enantiomers and stereoisomers, that is, both the stereomerically pure form (e.g., geometrically pure, enantiomerically pure, or diastereomerically pure) and enantiomeric and stereoisomeric mixtures.

The term “racemic mixture” denotes a mixture that is about 50% of one enantiomer and about 50% of the corresponding enantiomer relative to all chiral centers in the molecule. Thus, the invention encompasses all enantiomerically-pure, enantiomerically-enriched, and racemic mixtures of compounds of Formulas (I) through (VI).

Enantiomeric and stereoisomeric mixtures of compounds of the invention can be resolved into their component enantiomers or stereoisomers by well-known methods. Examples include, but are not limited to, the formation of chiral salts and the use of chiral or high performance liquid chromatography “HPLC” and the formation and crystallization of chiral salts. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); Wilen, S. H., Tables of Resolving Agents and Optical Resolutions p. 268 (E. L. Eliel, Ed., Univ. of Notre Dame Press, Notre Dame, Ind., 1972); Stereochemistry of Organic Compounds, Ernest L. Eliel, Samuel H. Wilen and Lewis N. Manda (1994 John Wiley & Sons, Inc.), and Stereoselective Synthesis A Practical Approach, Mihaly Nogradi (1995 VCH Publishers, Inc., NY, N.Y.). Enantiomers and stereoisomers can also be obtained from stereomerically- or enantiomerically-pure intermediates, reagents, and catalysts by well-known asymmetric synthetic methods.

“Substituted” is intended to indicate that one or more hydrogens on the atom indicated in the expression using “substituted” is replaced with a selection from the indicated group(s), provided that the indicated atom's normal valency is not exceeded, and that the substitution results in a stable compound. When a substituent is keto (i.e., ═O) group, then 2 hydrogens on the atom are replaced.

Unless moieties of a compound of the present invention are defined as being unsubstituted, the moieties of the compound may be substituted. In addition to any substituents provided above, the moieties of the compounds of the present invention may be optionally substituted with one or more groups independently selected from:

C₁-C₄ alkyl;

C₂-C₄ alkenyl;

C₂-C₄ alkynyl;

CF₃;

halo;

OH;

O—(C₁-C₄ alkyl);

OCH₂F;

OCHF₂;

OCF₃;

OC(O)—(C₁-C₄ alkyl);

OC(O)—(C₁-C₄ alkyl);

OC(O)NH—(C₁-C₄ alkyl);

OC(O)N(C₁-C₄ alkyl)₂;

OC(S)NH—(C₁-C₄ alkyl);

OC(S)N(C₁-C₄ alkyl)₂;

SH;

S—(C₁-C₄ alkyl);

S(O)—(C₁-C₄ alkyl);

S(O)₂—(C₁-C₄ alkyl);

SC(O)—(C₁-C₄ alkyl);

SC(O)O—(C₁-C₄ alkyl);

NH₂;

N(H)—(C₁-C₄ alkyl);

N(C₁-C₄ alkyl)₂;

N(H)C(O)—(C₁-C₄ alkyl);

N(CH₃)C(O)—(C₁-C₄ alkyl);

N(H)C(O)—CF₃;

N(CH₃)C(O)—CF₃;

N(H)C(S)—(C₁-C₄ akyl);

N(CH₃)C(S)—(C₁-C₄ alkyl);

N(H)S(O)₂—(C₁-C₄ alkyl);

N(H)C(O)NH₂;

N(H)C(O)NH—(C₁-C₄ alkyl);

N(CH₃)C(O)NH—(C₁-C₄ alkyl);

N(H)C(O)N(C₁-C₄ alkyl)₂;

N(CH₃)C(O)N(C₁-C₄ alkyl)₂;

N(H)S(O)₂NH₂);

N(H)S(O)₂NH—(C₁-C₄ alkyl);

N(CH₃)S(O)₂NH—(C₁-C₄ alkyl);

N(H)S(O)₂N(C₁-C₄ alkyl)₂;

N(CH₃)S(O)₂N(C₁-C₄ alkyl)₂;

N(H)C(O)O—(C₁-C₄ alkyl);

N(CH₃)C(O)O—(C₁-C₄ alkyl);

N(H)S(O)₂O—(C₁-C₄ alkyl);

N(CH₃)S(O)₂O—(C₁-C₄ alkyl);

N(CH₃)C(S)NH—(C₁-C₄ alkyl);

N(CH₃)C(S)N(C₁-C₄ alkyl)₂;

N(CH₃)C(S)O—(C₁-C₄ alkyl);

N(H)C(S)NH₂;

NO₂;

CO₂H;

CO₂—(C₁-C₄ alkyl);

C(O)N(H)OH;

C(O)N(CH₃)OH:

C(O)N(CH₃)OH;

C(O)N(CH₃)O—(C₁-C₄ alkyl);

C(O)N(H)—(C₁-C₄ alkyl);

C(O)N(C₁-C₄ alkyl)₂;

C(S)N(H)—(C₁-C₄ alkyl);

C(S)N(C₁-C₄ alkyl)₂;

C(NH)N(H)—(C₁-C₄ alkyl);

C(NH)N(C₁-C₄ alkyl)₂;

C(NCH₃)N(H)—(C₁-C₄ alkyl);

C(NCH₃)N(C₁-C₄ alkyl)₂;

C(O)—(C₁-C₄ alkyl);

C(NH)—(C₁-C₄ alkyl);

C(NCH₃)—(C₁-C₄ alkyl);

C(NOH)—(C₁-C₄ alkyl);

C(NOCH₃)—(C₁-C₄ alkyl);

CN;

CHO;

CH₂OH;

CH₂O—(C₁-C₄ alkyl);

CH₂NH₂;

CH₂N(H)—(C₁-C₄ alkyl);

CH₂N(C₁-C₄alkyl)₂;

aryl;

heteroaryl;

cycloalkyl; and

heterocyclyl.

In some cases, a ring substituent may be shown as being connected to the ring by a bond extending from the center of the ring. The number of such substituents present on a ring is indicated in subscript by a number. Moreover, the substituent may be present on any available ring atom, the available ring atom being any ring atom which bears a hydrogen which the ring substituent may replace. For illustrative purposes, if variable R^{x} were defined as being: 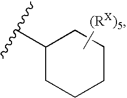
this would indicate a cyclohexyl ring bearing five R^{x} substituents. The R^{x} substituents may be bonded to any available ring atom. For example, among the configurations encompassed by this are configurations such as:

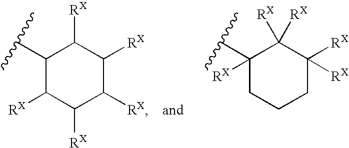

These configurations are illustrative and are not meant to limit the scope of the invention in any way.

In some embodiments of the present invention, the substituted bis-amide metalloprotease inhibiting compounds are represented by the general Formula (I):

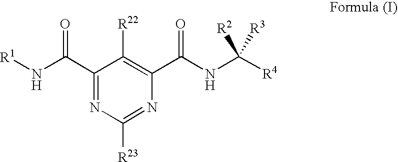

wherein:

R¹ is selected from the group consisting of hydrogen, alkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,

wherein R¹ is optionally substituted one or more times, or

wherein R¹ is optionally substituted by one R¹⁶ group and optionally substituted by one or more R⁹ groups;

R² is selected from the group consisting of hydrogen, alkyl, haloalkyl, fluoroalkyl, cycloalkyl, alkenyl, alkynyl, aryl, heteroaryl, cycloalkyl-alkyl, arylalkyl, heteroarylalkyl, COOR¹⁰, CONR¹⁰R¹¹, SO₂R¹⁰ and SO₂NR¹⁰R¹¹ wherein alkyl, haloalkyl, fluoroalkyl, cycloalkyl, alkenyl, alkynyl, aryl, heteroaryl, cycloalkyl-alkyl, arylalkyl, and heteroarylalkyl are optionally substituted one or more times;

R³ is selected from the group consisting of hydrogen, alkyl, haloalkyl, fluoroalkyl, cycloalkyl, alkenyl, alkynyl, aryl, heteroaryl, cycloalkyl-alkyl, arylalkyl, heteroarylalkyl, COOR¹⁰, CONR¹⁰R¹¹, SO₂R¹⁰ and SO₂NR¹⁰R¹¹ wherein alkyl, haloalkyl, fluoroalkyl, cycloalkyl, alkenyl, alkynyl, aryl, heteroaryl, cycloalkyl-alkyl, arylalkyl, and heteroarylalkyl are optionally substituted one or more times;

R⁴ is selected from the group consisting of alkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl, wherein R⁴ is optionally substituted one or more times;

R⁵ in each occurrence is independently selected from the group consisting of hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;

R⁹ in each occurrence is independently selected from the group consisting of R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)alkyl-P(O)₂OH, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)R¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,

wherein each R⁹ group is optionally substituted, or

wherein each R⁹ group is optionally substituted by one or more R¹⁴ groups;

R¹⁰ and R¹¹ are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted, or R¹⁰ and R¹¹ when taken together with the nitrogen to which they are attached complete a 3- to 8-membered ring containing carbon atoms and optionally containing a heteroatom selected from O, S, or NR⁵⁰ and which is optionally substituted;

R¹⁴ is independently selected from the group consisting of hydrogen, alkyl, arylalkyl, cycloalkylalkyl, heteroarylalkyl, heterocyclylalkyl and halo, wherein alkyl, arylalkyl, cycloalkylalkyl, heteroarylalkyl and heterocyclylalkyl are optionally substituted one or more times;

R¹⁶ is selected from the group consisting of cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii): 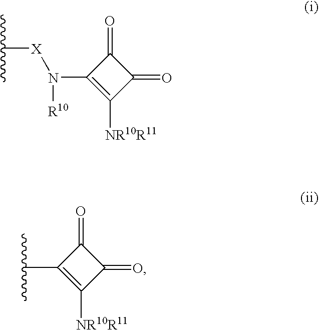
wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;

R²² and R²³ are independently selected from the group consisting of hydrogen, hydroxy, halo, alkyl, cycloalkyl, alkoxy, alkenyl, alkynyl, NO₂, NR¹⁰R¹¹, CN, SR¹⁰, SSR¹⁰, PO₃R¹⁰, NR¹⁰NR¹⁰R¹¹, NR¹⁰N═CR¹⁰R¹¹, NR¹⁰SO₂R¹¹, C(O)OR¹⁰, C(O)NR¹⁰R¹¹, SO₂R¹⁰, SO₂NR¹⁰R¹¹ and fluoroalkyl, wherein alkyl, cycloalkyl, alkoxy, alkenyl, alkynyl, and fluoroalkyl are optionally substituted one or more times;

R³⁰ is selected from the group consisting of alkyl and (C₀-C₆)-alkyl-aryl, wherein alkyl and aryl are optionally substituted;

R⁵⁰ is selected from the group consisting of hydrogen, alkyl, aryl, heteroaryl, C(O)R¹⁰, C(O)NR¹⁰R¹¹, SO₂R¹⁰ and SO₂NR¹⁰R¹¹, wherein alkyl, aryl, and heteroaryl are optionally substituted;

E is selected from the group consisting of a bond, CR¹⁰R¹¹, O, NR⁵, S, S═O, S(═O)₂, C(═O), N(R¹⁰(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and

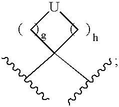

U is selected from the group consisting of C(R⁵R¹⁰), NR⁵, O, S, S═O and S(═O)₂;

W¹ is selected from the group consisting of O, NR⁵, S, S═O, S(═O)₂, N(R¹⁰)(C═O), N(R¹⁰)S(═O)₂ and S(═O)₂N(R¹⁰);

X is selected from the group consisting of a bond and (CR₁₀R¹¹)_{w}E(CR₁₀R¹¹)_{w};

g and h are independently selected from 0-2;

w is independently selected from 0-4;

x is selected from 0 to 2;

y is selected from 1 and 2;

with the proviso that R² and R³ are not both hydrogen.

In some embodiments of the present invention R¹ may be:

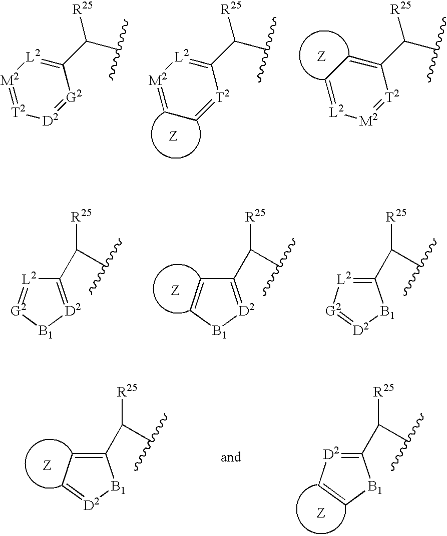

wherein:

R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, heteroaryl are optionally substituted one or more times;

R²⁵ is selected from the group consisting of hydrogen, alkyl, cycloalkyl, CO₂R¹⁰, C(O)NR¹⁰R¹¹ and haloalkyl, wherein alkyl, cycloalkyl, and haloalkyl are optionally substituted one or more times;

B₁ is selected from the group consisting of NR¹⁰, O and S(O)ₓ;

D², G², L², M² and T² are independently selected from the group consisting of CR¹⁸ and N; and

Z is a 5- to 8-membered ring selected from the group consisting of cycloalkyl, heterocycloalkyl, or a 5- to 6-membered ring selected from the group consisting of aryl and heteroaryl, wherein cycloalkyl, heterocycloalkyl, aryl and heteroaryl are optionally substituted one or more times.

More specifically, R¹ may be, but is not limited to, the following:

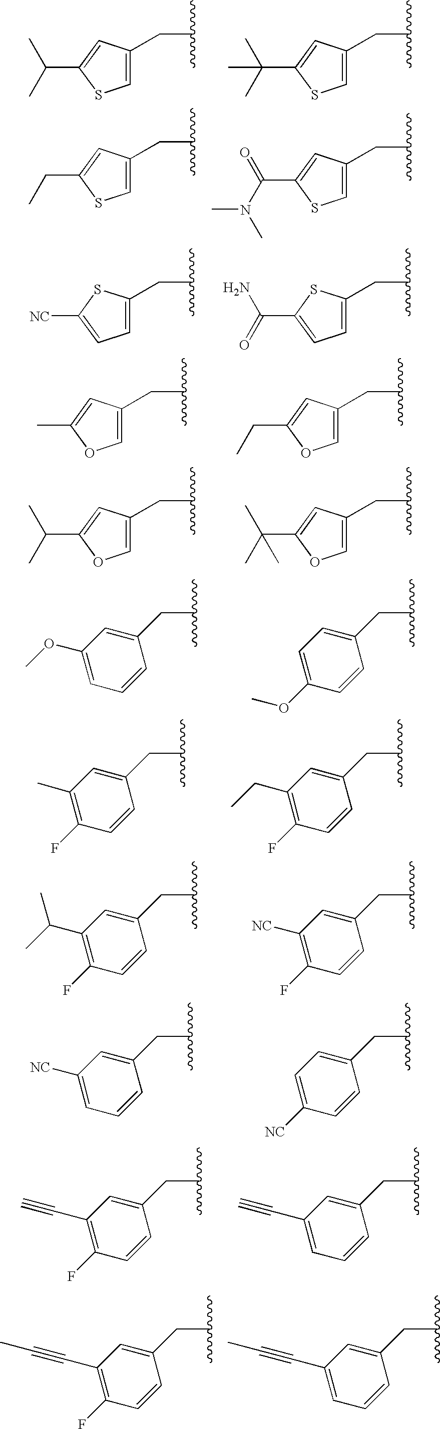

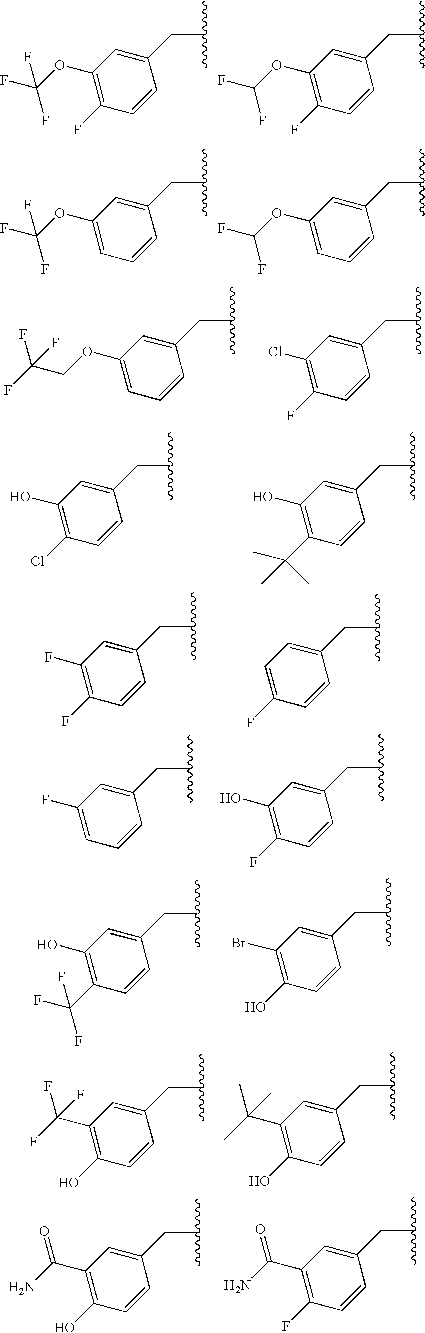

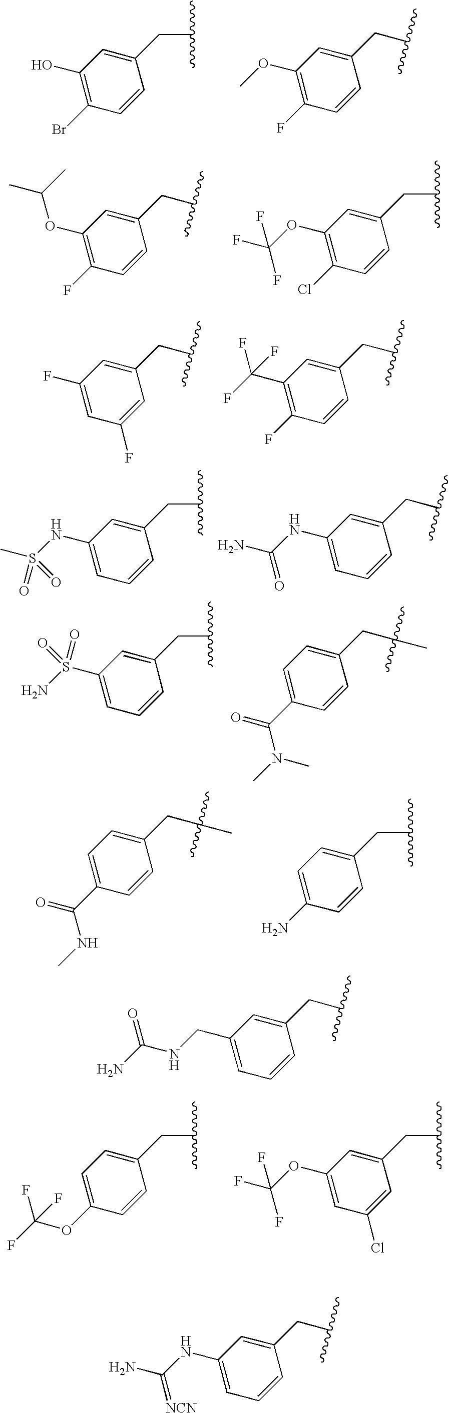

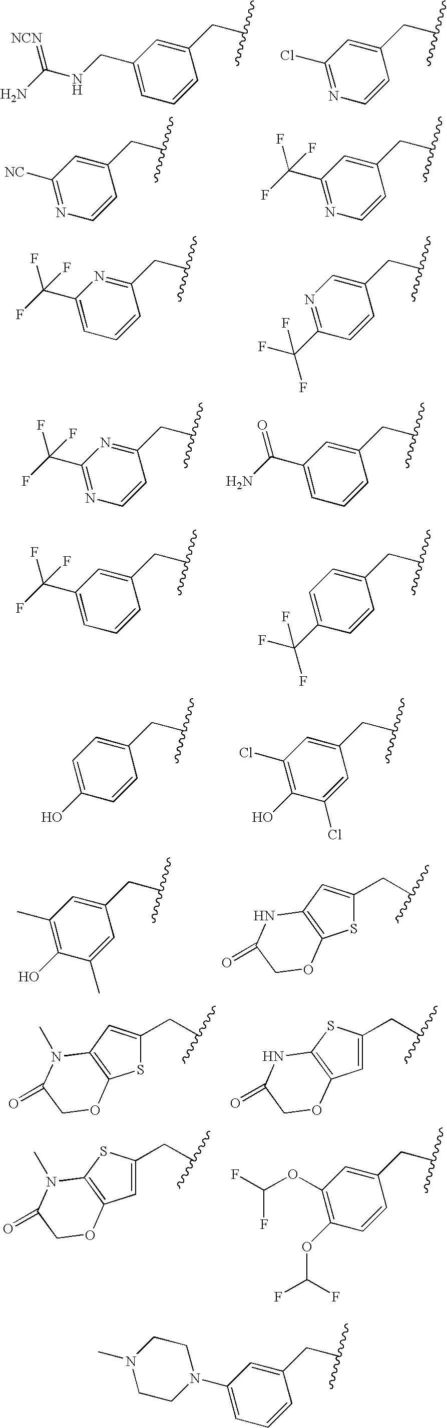

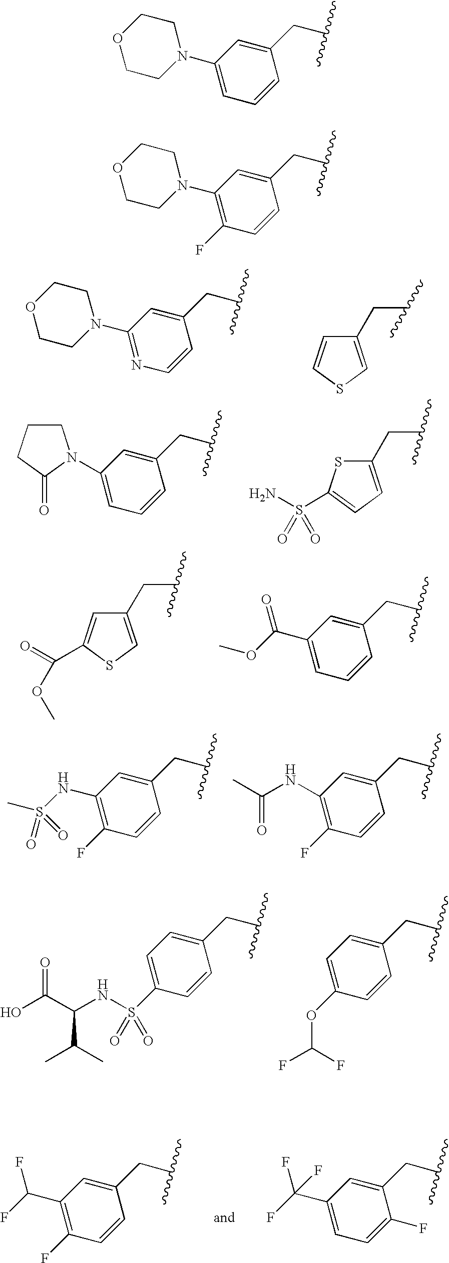

Alternatively, in some embodiments of the present invention, R¹ may include a bicyclic ring system. In such embodiments, R¹ may be:

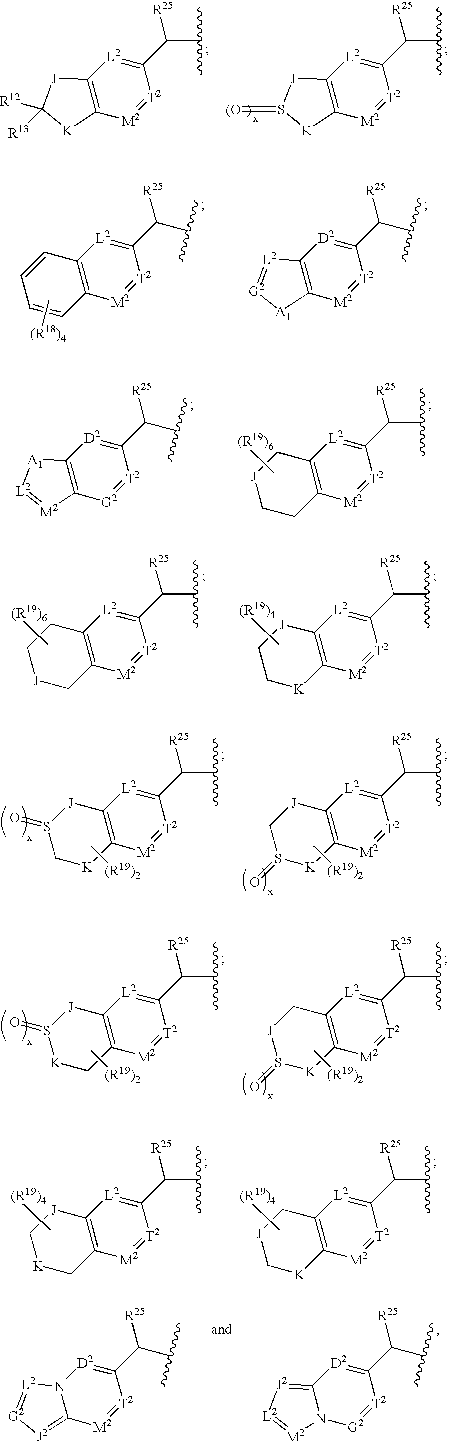

wherein:

R¹² and R¹³ are independently selected from the group consisting of hydrogen, alkyl and halo, wherein alkyl is optionally substituted one or more times, or optionally R¹² and R¹³ together form ═O, ═S or R═NR¹⁰;

R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;

R¹⁹ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;

R²⁵ is selected from the group consisting of hydrogen, alkyl, cycloalkyl, CO₂R¹⁰, C(O)NR¹⁰R¹¹ and haloalkyl, wherein alkyl, cycloalkyl, and haloalkyl are optionally substituted one or more times;

J and K are independently selected from the group consisting of CR¹⁰R¹⁸, NR¹⁰, O and S(O)ₓ;

A₁ is selected from the group consisting of NR¹⁰, O and S(O)ₓ; and

D², G², J², L², M² and T² are independently selected from the group consisting of CR¹⁸ and N.

More specifically, R¹ may be, but is not limited to, the following:

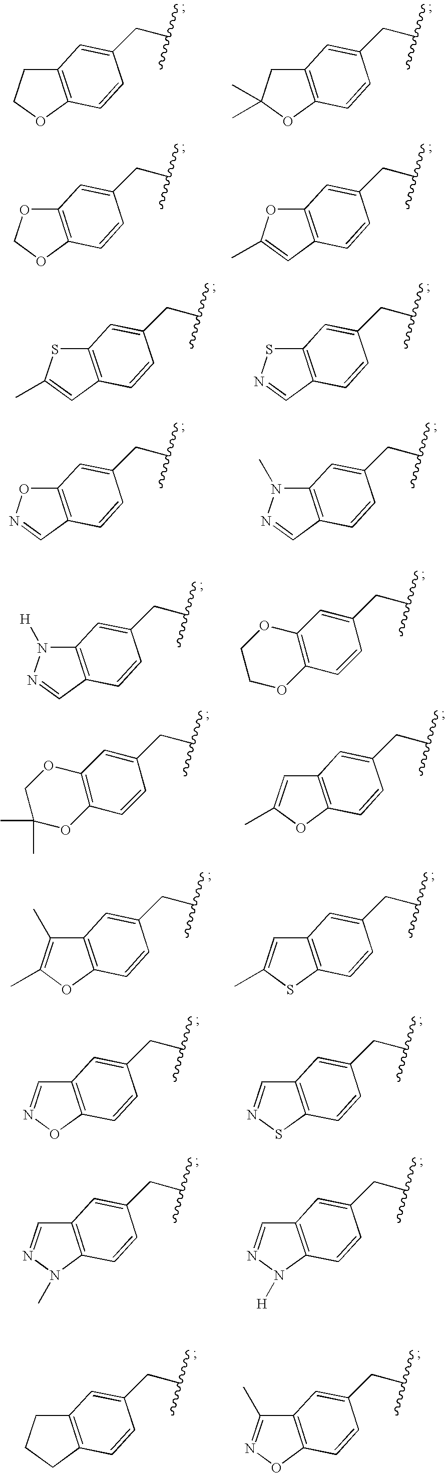

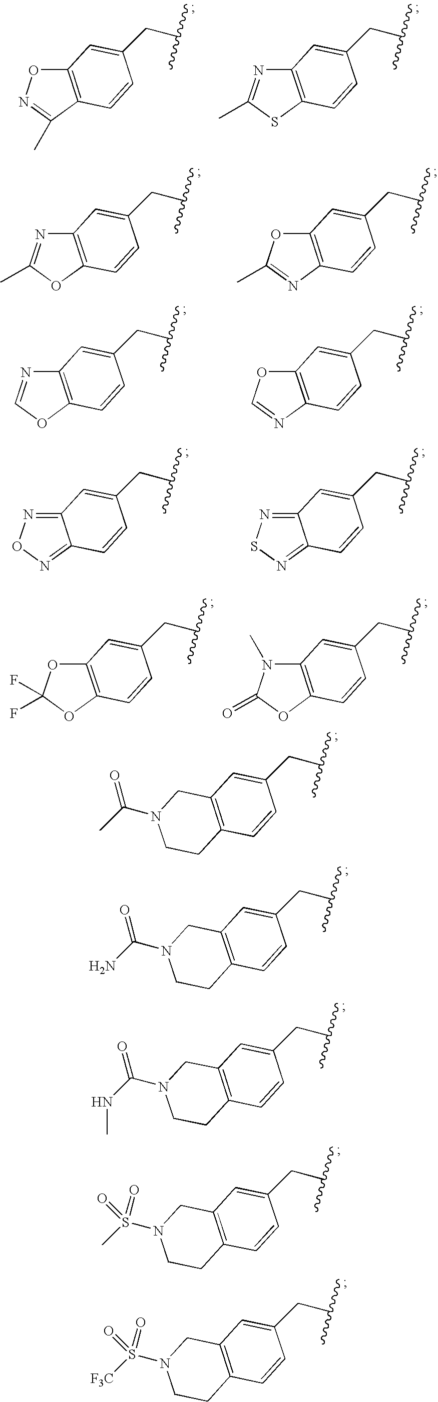

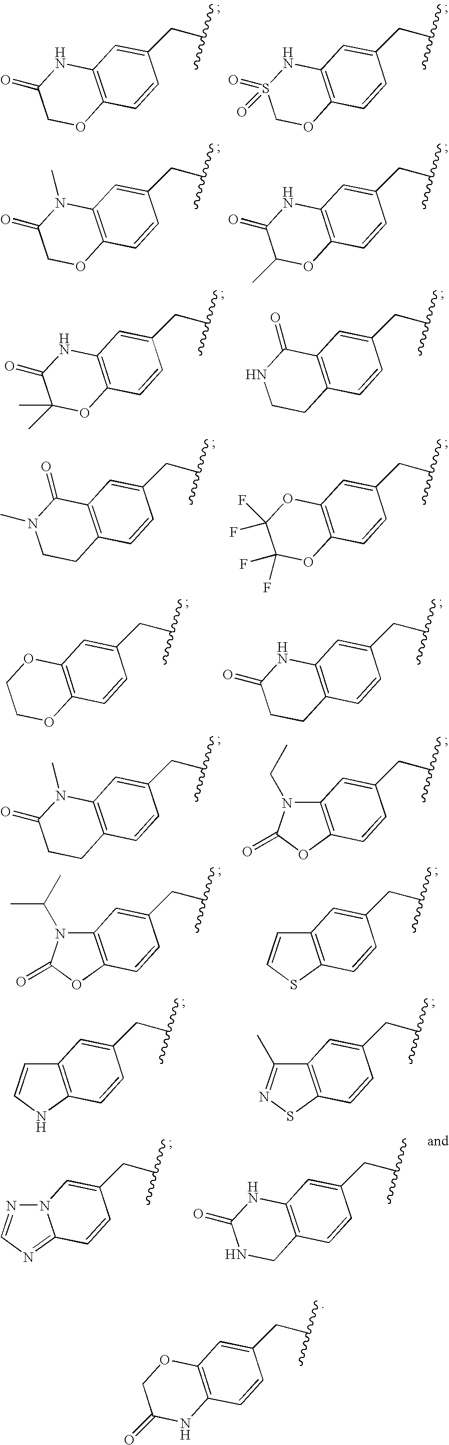

In some embodiments of the present invention, R¹ may be:

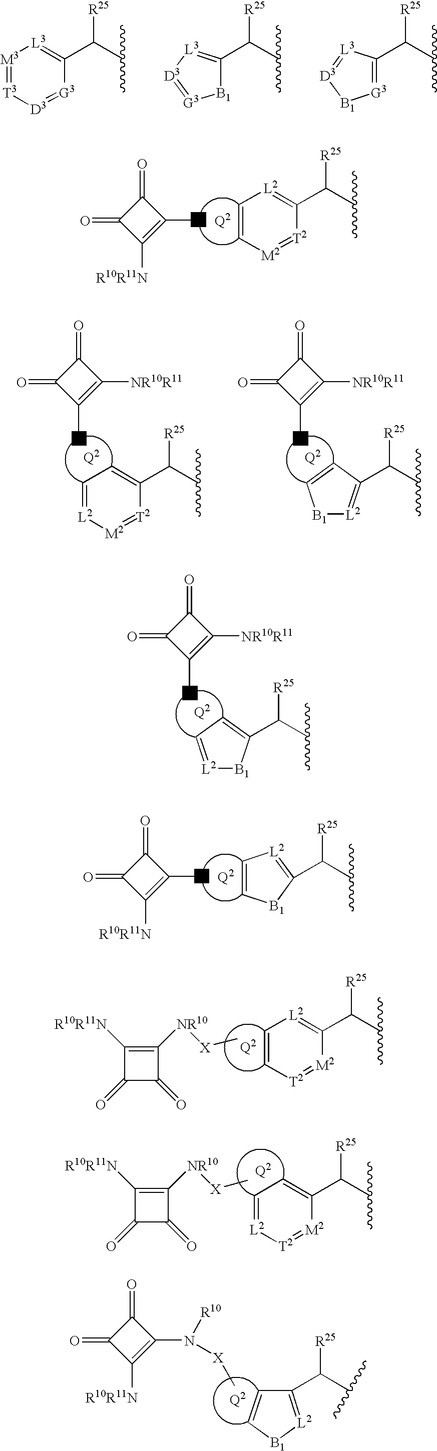

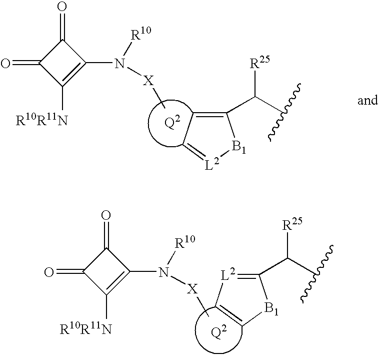

wherein:

R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;

R¹⁹ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;

R²⁵ is selected from the group consisting of hydrogen, alkyl, cycloalkyl, CONR¹⁰R¹¹ and haloalkyl, wherein alkyl, cycloalkyl and haloalkyl are optionally substituted one or more times;

L², M², and T² are independently selected from the group consisting of CR¹⁸ and N;

D³, G³, L³, M³, and T³ are independently selected from N, CR¹⁸, (i), or (ii),

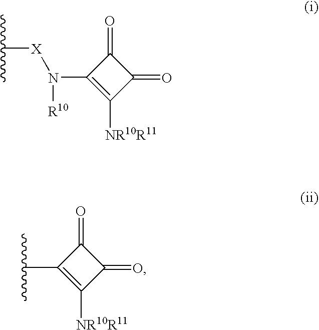

with the proviso that one of L³, M³, T³, D³, and G is (i) or (ii);

B₁ is selected from the group consisting of NR¹⁰, O and S(O)ₓ; and

Q² is a 5- to 8-membered ring selected from the group consisting of cycloalkyl, heterocycloalkyl, aryl, and heteroaryl, which is optionally substituted one or more times with R¹⁹.

More specifically, R¹ may be, but is not limited to, the following:

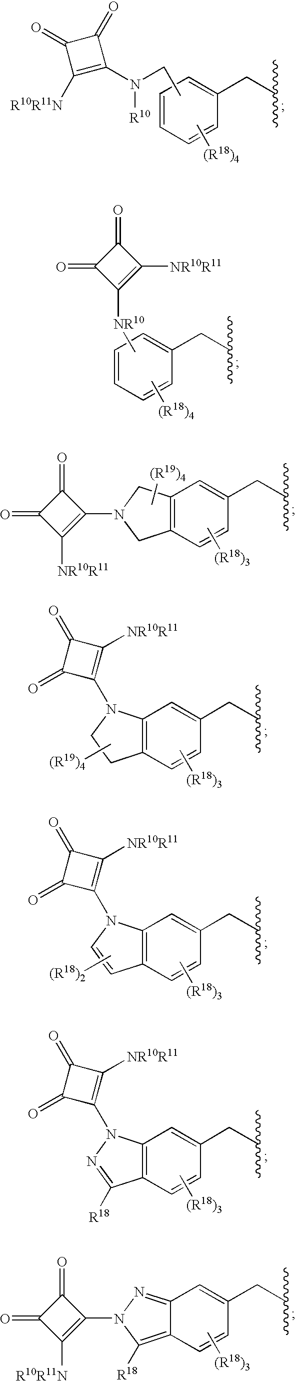

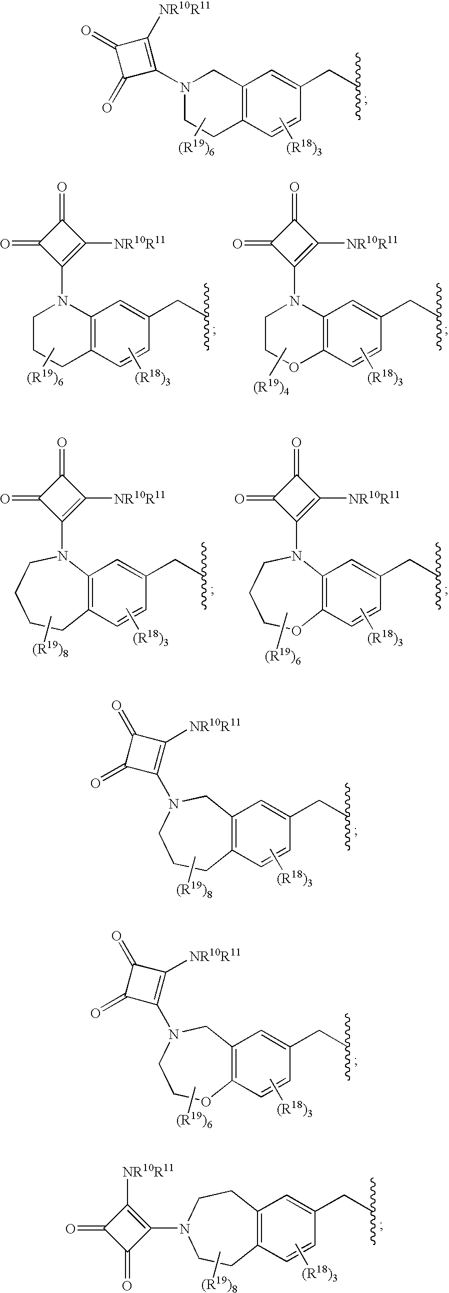

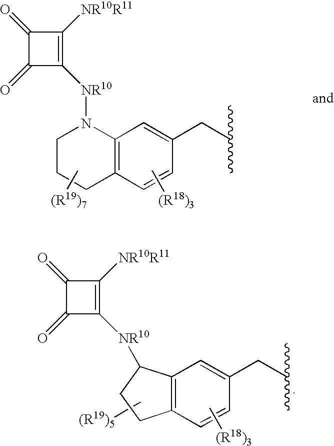

More specifically, R¹ may be, but is not limited to, the following:

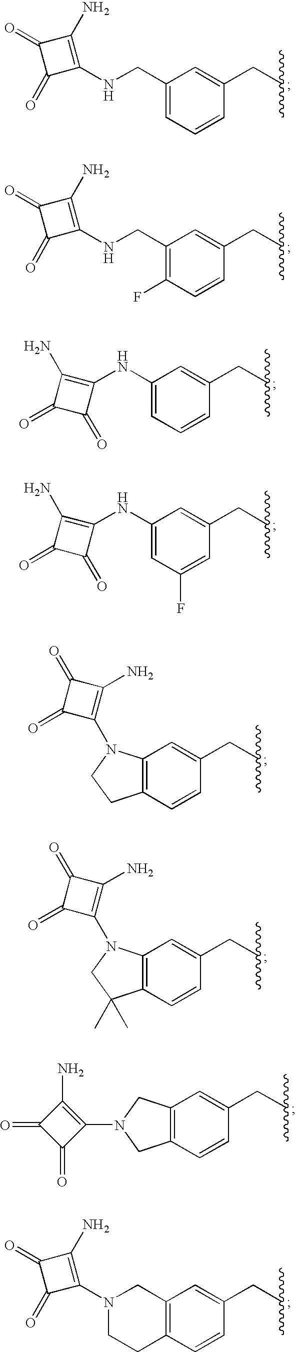

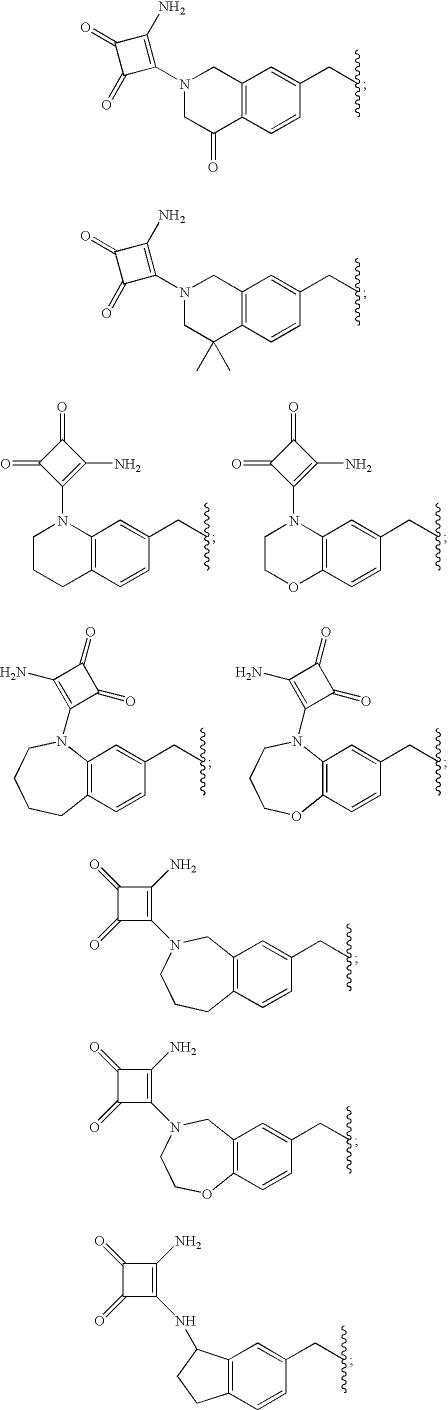

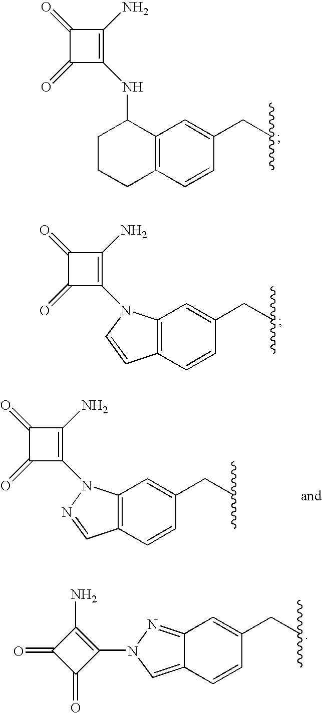

In some embodiments of the present invention,

R² is selected from the group consisting of alkyl, haloalkyl, fluoroalkyl, cycloalkyl, alkenyl, alkynyl, aryl, heteroaryl, cycloalkyl-alkyl, arylalkyl, heteroarylalkyl, COOR¹⁰, CONR¹⁰R¹¹, SO₂R¹⁰ and SO₂NR¹⁰R¹¹ wherein alkyl, haloalkyl, fluoroalkyl, cycloalkyl, alkenyl, alkynyl, aryl, heteroaryl, cycloalkyl-alkyl, arylalkyl, and heteroarylalkyl are optionally substituted one or more times; and

R³ is hydrogen.

More specifically, but not limiting to

R² is selected from the group consisting of alkyl, haloalkyl, fluoroalkyl, COOR¹⁰, CONR¹⁰R¹¹, wherein alkyl, haloalkyl, fluoroalkyl are optionally substituted one or more times; and

R³ is hydrogen.

Even more specifically, but not limiting to

R² is alkyl, which is optionally substituted one or more times; and

R³ is hydrogen.

In some embodiments of the present invention, R⁴ may be:

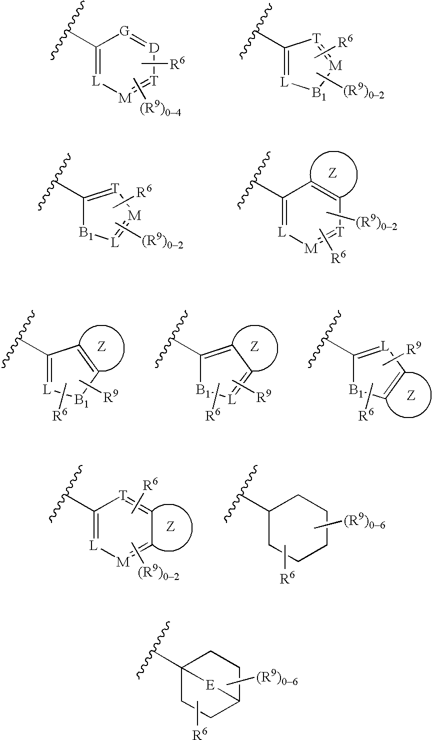

wherein

R⁶ is independently selected from the group consisting of R⁹, alkenyl, alkynyl, cycloalkyl, heterocycloalkyl, bicycloalkyl, heterobicycloalkyl, spiroalkyl, spiroheteroalkyl, aryl, heteroaryl, C(O)OR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl, wherein each R⁶ group is optionally substituted by one or more R¹⁴ groups;

B₁ is selected from NR¹⁰, O or S(O)ₓ;

L, M, T, D and G are independently selected from C or N;

Z is a 5- to 8-membered ring selected from the group consisting of cycloalkyl, heterocycloalkyl, or a 5- to 6-membered ring selected from the group consisting of aryl and heteroaryl, wherein cycloalkyl, heterocycloalkyl, aryl and heteroaryl are optionally substituted one or more times.

More specifically, in such embodiments, R⁴ may be, but is not limited to, the following:

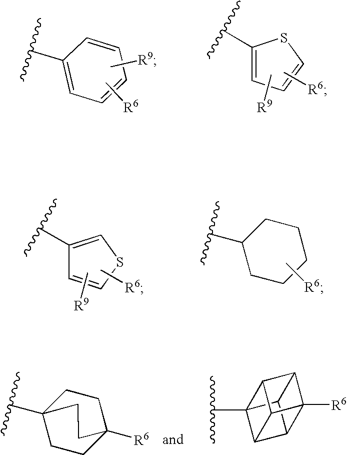

wherein

R⁶ is selected from the group consisting of

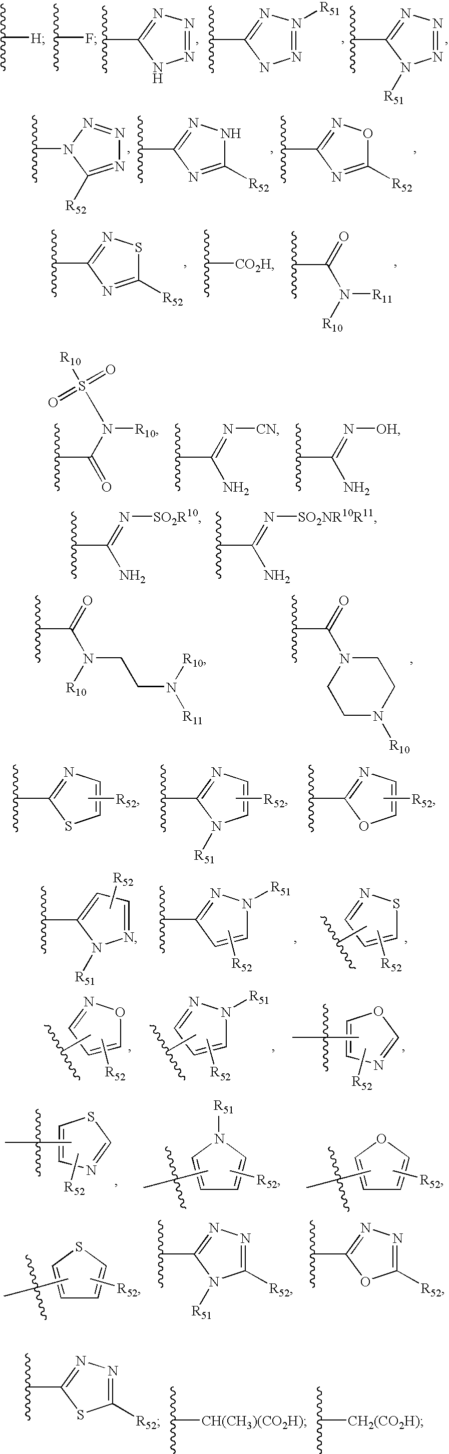

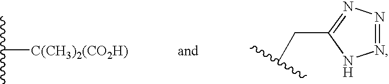

R⁹ is selected from the group consisting of hydrogen, alkyl, halo, CF₃, COR¹⁰, OR¹⁰, NR¹⁰R¹¹, NO₂, CN, wherein alkyl is optionally substituted;

R⁵¹ is selected from the group consisting of hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted;

R⁵² is selected from the group consisting of hydrogen, halo, hydroxy, alkoxy, fluoroalkoxy, alkyl, aryl, heteroaryl, arylalkyl, cycloalkylalkyl, heteroarylalkyl, haloalkyl, C(O)NR¹⁰R¹¹ and SO₂NR¹⁰R¹¹, wherein alkoxy, fluoroalkoxy, alkyl, aryl, heteroaryl, arylalkyl, cycloalkylalkyl, heteroarylalkyl and haloalkyl are optionally substituted.

In accordance with some embodiments of the present invention, R⁶ may be COOH or heteroaryl. More specifically, in some embodiments R⁶ may be: COOH, dioxole, imidazole, furan, thiazole, isothiazole, isoxazole, morpholine, 1,2,4-oxadiazole, 1,3,4-oxadiazole, 1,2,4-oxadiazole, 1,2-oxazine, 1,3-oxazine, 1,4-oxazine, oxirane, oxazole, 5-oxo-1,2,4-oxadiazole, 5-oxo-1,2,4-thiadiazole, piperzine, piperidine, pyran, pyrazine, pyrazole, pyridazine, pyridine, pyrimidine, pyrrole, pyrrolidine, tetrazine, tetrazole, thiazine, 1,2,3-thiadiazole, 1,2,4-thiadiazole, 1,3,4-thiadiazole, 1,2,5-thiadiazole, thiatriazole, 1,2-thiazine, 1,3-thiazine, 1,4-thiazine, thiazole, 5-thioxo-1,2,4-diazole, thiomorpholine, thiophene, thiopyran, 1,2,3-triazine, 1,2,4-triazine, 1,3,5-triazine, 1,2,4-triazole, 1,2,3-triazole or triazolones, which are optionally substituted.

More specifically, in such embodiments, R⁴ may be, but is not limited to, the following:

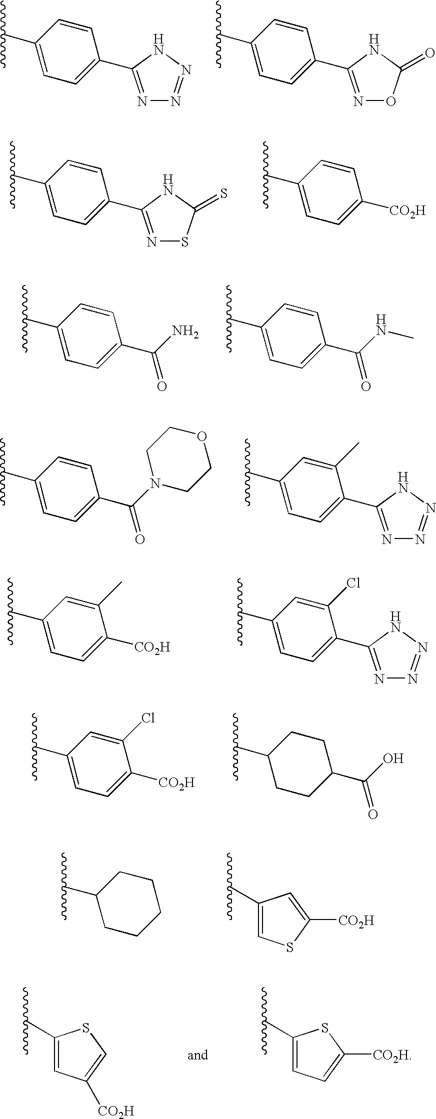

More specifically, the compounds of Formula (I) may be selected from, but are not limited to, the following:

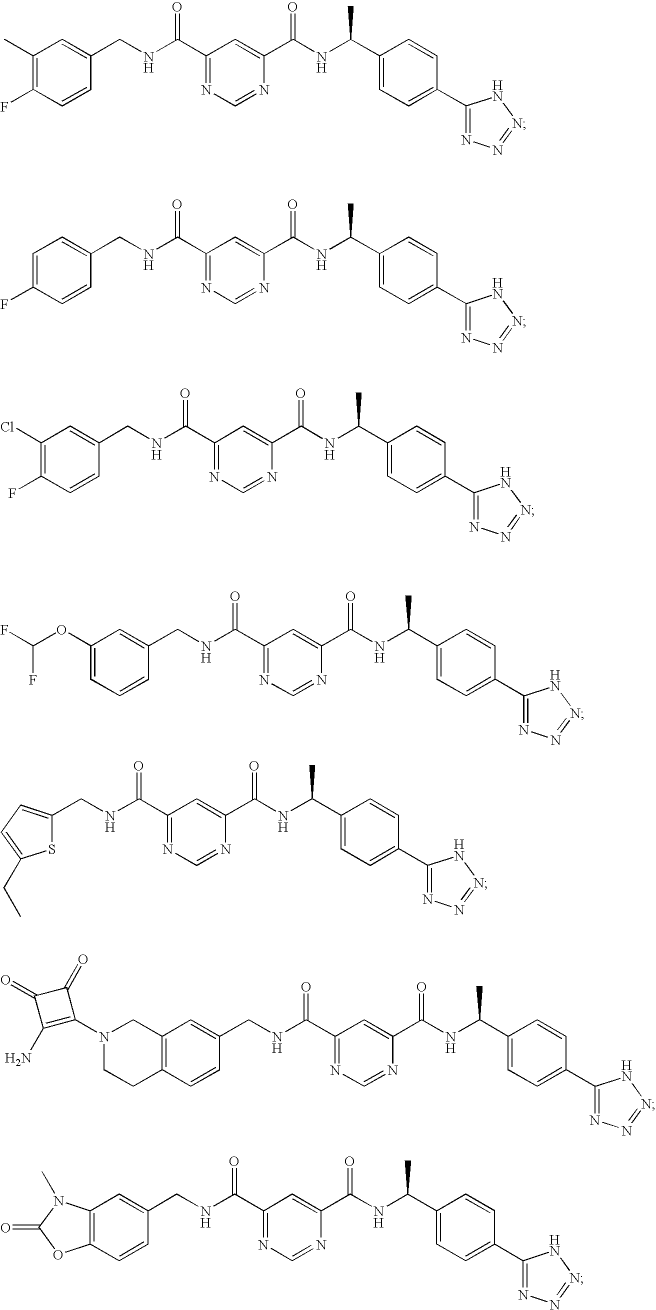

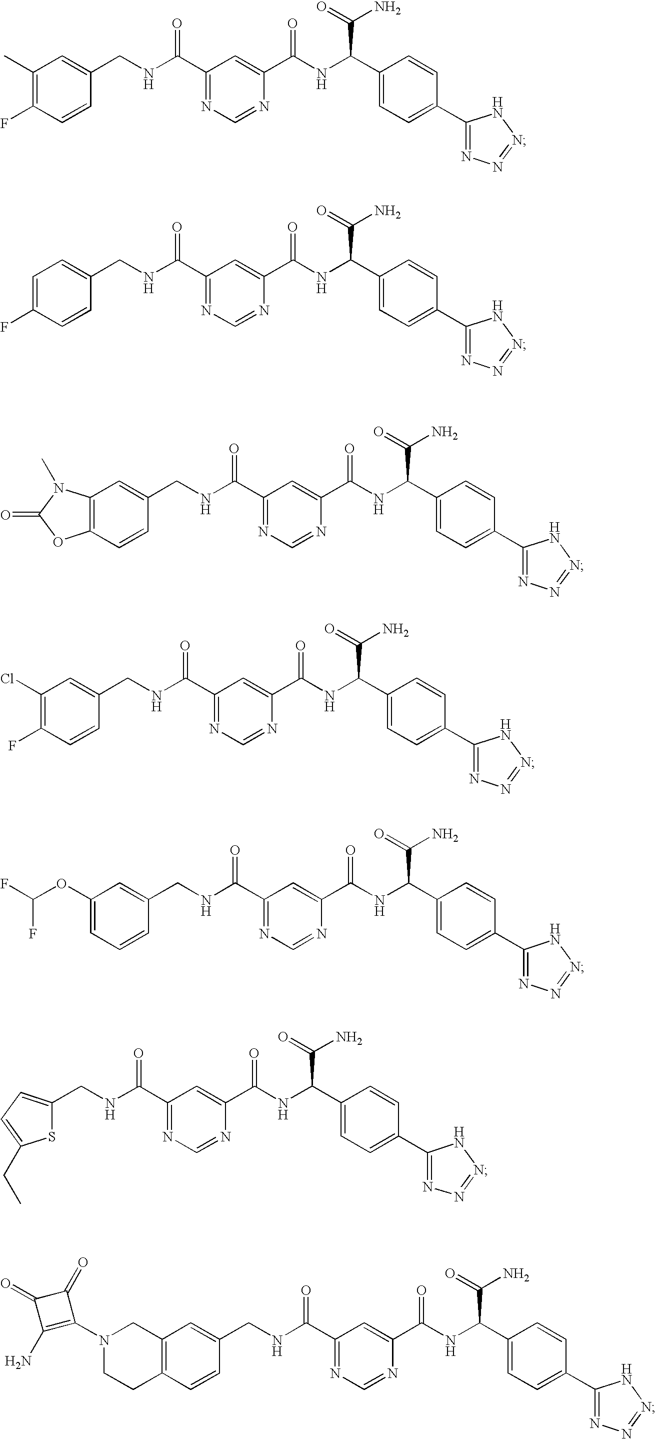

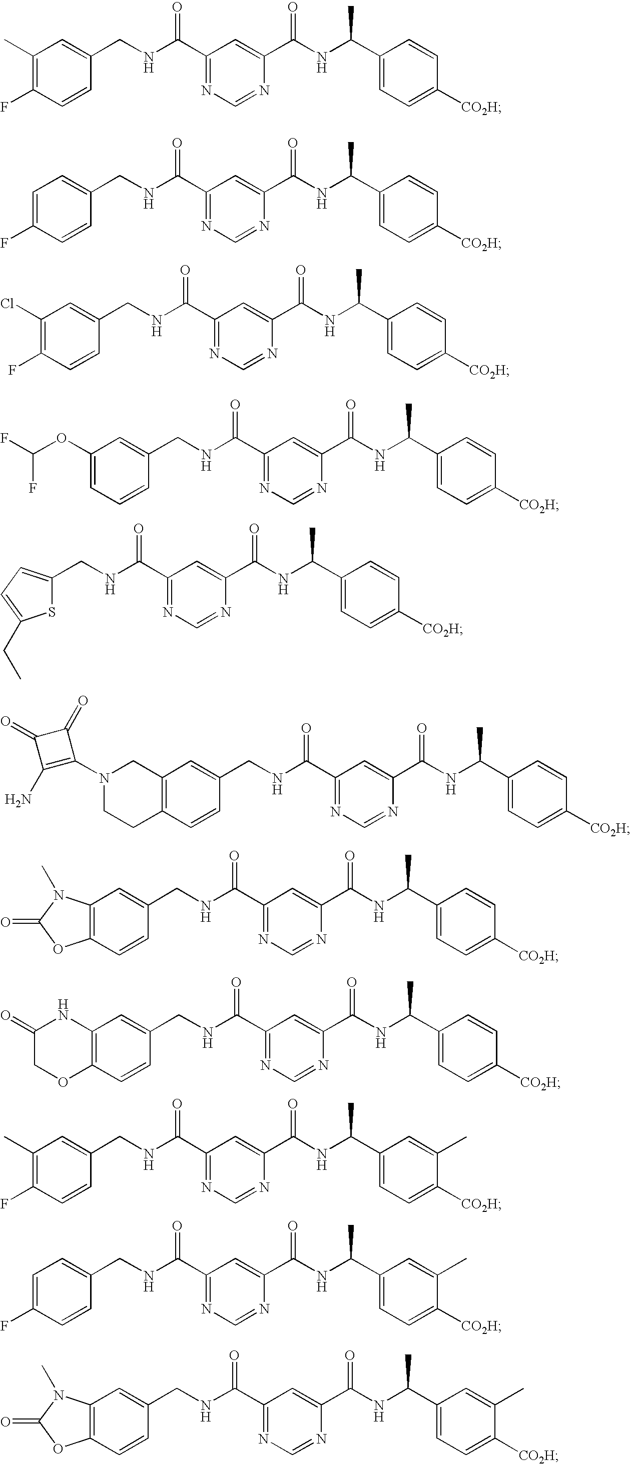

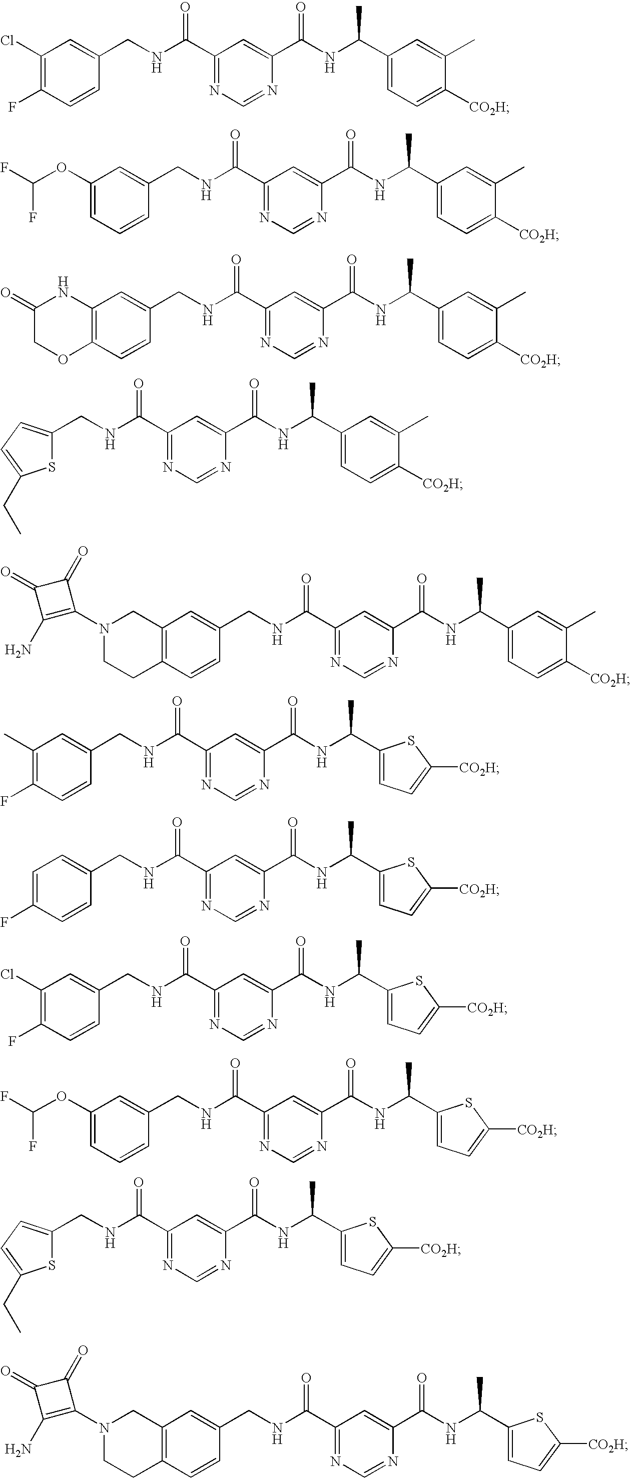

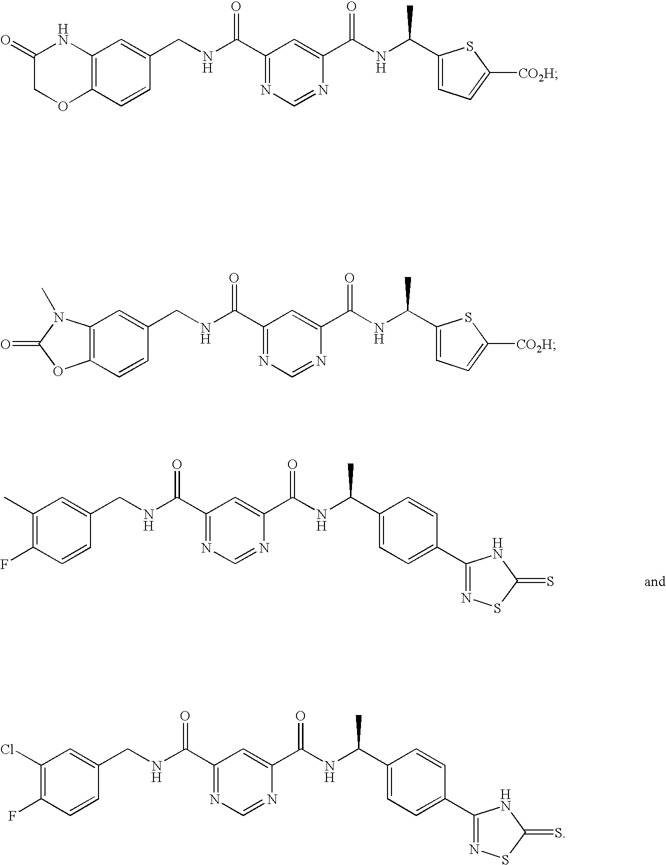

The substituent variables employed in the above Formulas may be further defined as provided in Assignee's co-pending U.S. patent application, entitled “Multicyclic Bis-Amide MMP Inhibitors,” filed on Dec. 30, 2005 (Express Mail Label No. EV706432935US), which definitions are incorporated by reference herein.

It is contemplated that the compounds of the present invention represented by the Formulas described above include all diastereomers and enantiomers, as well as racemic mixtures. Racemic mixtures may be separated by chiral salt resolution or by chiral column HPLC chromatography.

The present invention also is directed to pharmaceutical compositions including any of the substituted bis-amide metalloprotease inhibiting compounds of the present invention described above. In accordance therewith, some embodiments of the present invention provide a pharmaceutical composition which may include an effective amount of a substituted bis-amide metalloprotease inhibiting compound of the present invention and a pharmaceutically acceptable carrier.

The present invention also is directed to methods of inhibiting metalloproteases, in particular MMP-13 and methods of treating diseases or symptoms mediated by an metalloprotease enzyme, in particular an MMP-13 enzyme. Such methods include administering a substituted bis-amide metalloprotease inhibiting compound of the present invention, such as a compound of Formula (I), as defined above, or a pharmaceutically acceptable salt thereof. Examples of diseases or symptoms mediated by an metalloprotease mediated enzyme—in particular the MMP-13 enzyme—include, but are not limited to, rheumatoid arthritis, osteoarthritis, abdominal aortic aneurysm, cancer (e.g. but not limited to melanoma, gastric carcinoma or non-small cell lung carcinoma), inflammation, atherosclerosis, multiple sclerosis, chronic obstructive pulmonary disease, ocular diseases (e.g. but not limited to ocular inflammation, retinopathy of prematurity, macular degeneration with the wet type preferred and corneal neovascularization), neurologic diseases, psychiatric diseases, thrombosis, bacterial infection, Parkinson's disease, fatigue, tremor, diabetic retinopathy, vascular diseases of the retina, aging, dementia, cardiomyopathy, renal tubular impairment, diabetes, psychosis, dyskinesia, pigmentary abnormalities, deafness, inflammatory and fibrotic syndromes, intestinal bowel syndrome, allergies, Alzheimers disease, arterial plaque formation, oncology, periodontal, viral infection, stroke, atherosclerosis, cardiovascular disease, reperfusion injury, trauma, chemical exposure or oxidative damage to tissues, wound healing, hemorroid, skin beautifying, pain, inflammatory pain, bone pain and joint pain, acne, acute alcoholic hepatitis, acute inflammation, acute pancreatitis, acute respiratory distress syndrome, adult respiratory disease, airflow obstruction, airway hyperresponsiveness, alcoholic liver disease, allograft rejections, angiogenesis, angiogenic ocular disease, arthritis, asthma, atopic dermatitis, bronchiectasis, bronchiolitis, bronchiolitis obliterans, burn therapy, cardiac and renal reperfusion injury, celiac disease, cerebral and cardiac ischemia, CNS tumors, CNS vasculitis, colds, contusions, cor pulmonae, cough, Crohn's disease, chronic bronchitis, chronic inflammation, chronic pancreatitis, chronic sinusitis, crystal induced arthritis, cystic fibrosis, delayted type hypersensitivity reaction, duodenal ulcers, dyspnea, early transplantation rejection, emphysema, encephalitis, endotoxic shock, esophagitis, gastric ulcers, gingivitis, glomerulonephritis, glossitis, gout, graft vs. host reaction, gram negative sepsis, granulocytic ehrlichiosis, hepatitis viruses, herpes, herpes viruses, HIV, hypercapnea, hyperinflation, hyperoxia-induced inflammation, hypoxia, hypersensitivity, hypoxemia, inflammatory bowel disease, interstitial pneumonitis, ischemia reperfusion injury, kaposi's sarcoma associated virus, lupus, malaria, meningitis, multi-organ dysfunction, necrotizing enterocolitis, osteoporosis, periodontitis, peritonitis associated with continous ambulatory peritoneal dialysis (CAPD), pre-term labor, polymyositis, post surgical trauma, pruritis, psoriasis, psoriatic arthritis, pulmatory fibrosis, pulmatory hypertension, renal reperfusion injury, respiratory viruses, restinosis, right ventricular hypertrophy, sarcoidosis, septic shock, small airway disease, sprains, strains, subarachnoid hemorrhage, surgical lung volume reduction, thrombosis, toxic shock syndrome, transplant reperfusion injury, traumatic brain injury, ulcerative colitis, vasculitis, ventilation-perfusion mismatching, and wheeze.

In some embodiments of the present invention, the substituted bis-amide metalloprotease inhibiting compounds defined above are used in the manufacture of a medicament for the treatment of a disease mediated by a metalloprotease enzyme, in particular an MMP-13 enzyme.

In some embodiments, the substituted bis-amide metalloprotease inhibiting compounds defined above may be used in combination with a drug, agent or therapeutic such as, but not limited to: (a) a disease modifying antirheumatic drug; (b) a nonsteroidal anti-inflammatory drug; (c) a COX-2 selective inhibitor; (d) a COX-1 inhibitor; (e) an immunosuppressive; (f) a steroid; (g) a biological response modifier; or (h) other anti-inflammatory agents or therapeutics useful for the treatment of chemokine mediated diseases.

Examples of disease modifying antirheumatic drugs include, but are not limited to, methotrexate, azathioptrineluflunomide, penicillamine, gold salts, mycophenolate, mofetil and cyclophosphamide.

Examples of nonsteroidal anitinflammatory drugs include, but are not limited to, piroxicam, ketoprofen, naproxen, indomethacin, and ibuprofen.

Examples of COX-2 selective inhibitors include, but are not limited to, rofecoxib, celecoxib, and valdecoxib.

An example of a COX-1 inhibitor includes, but is not limited to, piroxicam.

Examples of immunosuppressives include, but are not limited to, methotrexate, cyclosporin, leflunimide, tacrolimus, rapamycin and sulfasalazine.

Examples of steroids include, but are not limited to, p-methasone, prednisone, cortisone, prednisolone and dexamethasone.

Examples of biological response modifiers include, but are not limited to, anti-TNF antibodies, TNF-α antagonists, IL-1 antagonists, anti-CD40, anti-CD28, IL-10 and anti-adhesion molecules.

Examples of anti-inflammatory agents or therapeutics include, but are not limited to, p38 kinase inhibitors, PDE4 inhibitors, TACE inhibitors, chemokine receptor antagonists, thalidomide, leukotriene inhibitors and other small molecule inhibitors of pro-inflammatory cytokine production.

In accordance with another embodiment of the present invention, a pharmaceutical composition may include an effective amount of a compound of the present invention, a pharmaceutically acceptable carrier and a drug, agent or therapeutic selected from: (a) a disease modifying antirheumatic drug; (b) a nonsteroidal anti-inflammatory drug; (c) a COX-2 selective inhibitor; (d) a COX-1 inhibitor; (e) an immunosuppressive; (f) a steroid; (g) a biological response modifier; or (h) other anti-inflammatory agents or therapeutics useful for the treatment of chemokine mediated diseases.

In some embodiments of the present invention, the compounds of Formula (I) are synthesized by the general method shown in Scheme 1.

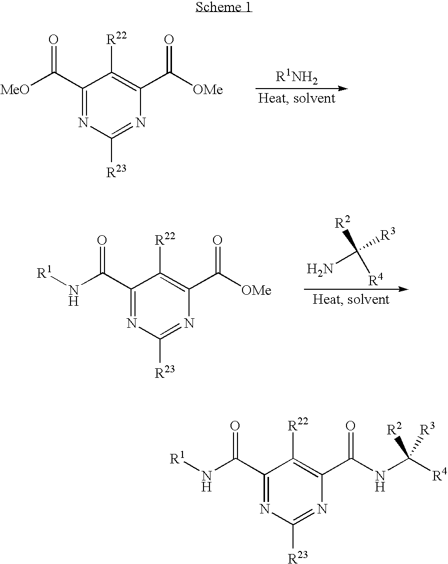

Dimethylpyrimidine-4,6-dicarboxylate (R²²═R²³═H) is treated with a slight molar excess of R¹NH₂ in a suitable solvent and heated to afford the desired adduct after purification. This compound is further treated with a slight molar excess of NH₂CR²R³R⁴ in a suitable solvent and heated to give the final desired adduct after purification. Alternatively, the final adduct can be obtained by one skilled in the art through comparable coupling reactions.

In some embodiments the compounds of Formula I are synthesized by the general method shown in Scheme 2.

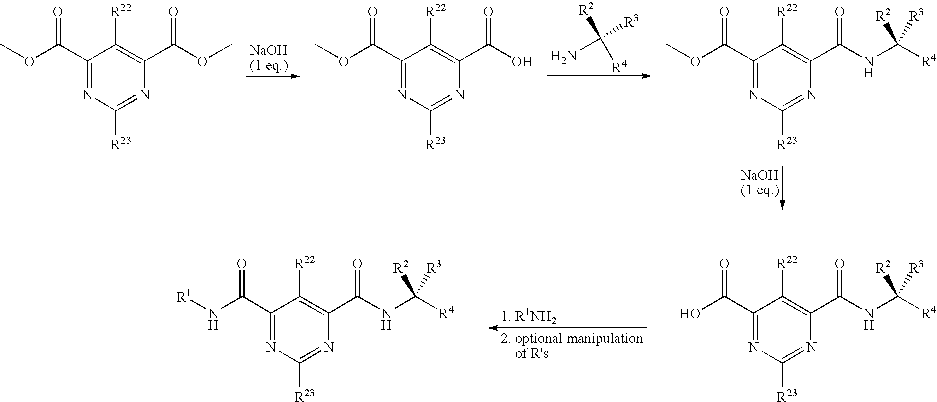

A dimethylpyrimidine-4,6-dicarboxylate derivative is treated with one equivalent sodium hydroxide to give the monomethylpyrimidine-4,6-dicarboxylate derivative. After an activated acid coupling (e.g. HOBt/EDCI, HOAt/HATU, PyBroP or ethyl chloroformate) of NH₂CR²R³R⁴ in a suitable solvent afford the desired adduct after purification. This compound is further treated with one equivalent sodium hydroxide and then coupled via an activated acid (e.g. HOBt/EDCI, HOAt/HATO, PyBroP or ethyl chloroformate) with R¹NH₂ to give the pyriridine-4,6-bis-amide. If necessary, the R group can be further manipulated (e.g. saponification of a COOMe group in R).

The MMP-13 inhibiting activity of the bis-amide metalloprotease inhibiting compounds of the present invention may be measured using any suitable assay known in the art. A standard in vitro assay for MMP-13 inhibiting activity is described in Example 999 and a description of the microsomal stability assay is described in Example 999a.

The bis-amide metalloprotease inhibiting compounds of the invention have an MMP-13 inhibition activity (IC₅₀ MMP-13) ranging from about 1 nM to about 20 μM, and typically, from about 8 nM to about 2 μM. Bis-amide metalloprotease inhibiting compounds of the invention desirably have an MMP inhibition activity ranging from about 1 nM to about 20 nM. Table 1 lists typical examples of bis-amide metalloprotease inhibiting compounds of the invention that have an MMP-13 activity lower than about 1 μM, particularly about 1 nM to 300 nM, and more specifically about 1 nM to 50 nM.

TABLE 1
Summary of MMP-13 Activity for Compounds of Formula I

| Ex. # | Structure | IC₅₀ (nM) |
|---|---|---|
| 1000 | 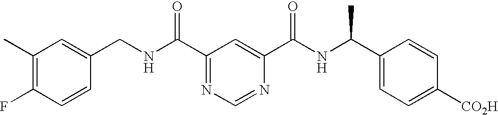 | <200 |
| 1007 | 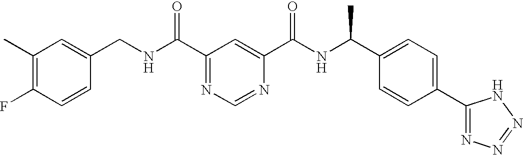 | <10 |
| 1007d | 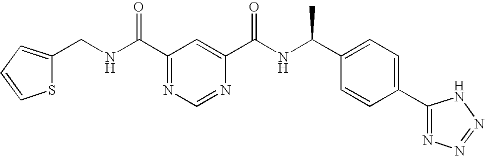 | <1000 |
| 1007o | 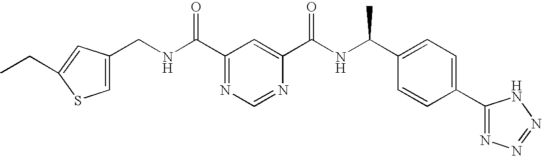 | <200 |
| 1014 | 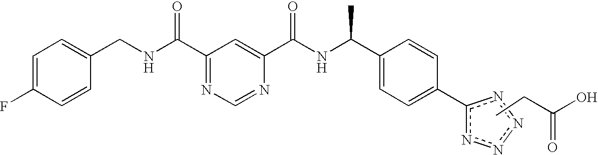 | <1000 |
| 1021 | 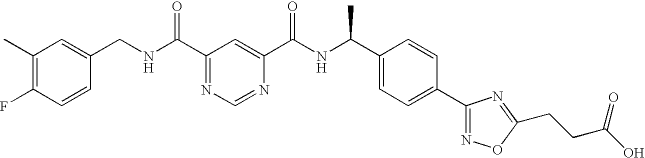 | <200 |
| 1028e | 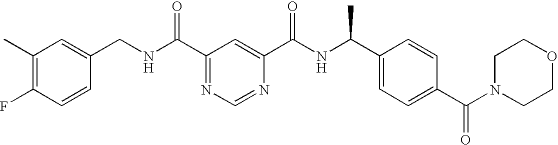 | <200 |
| 1002 | 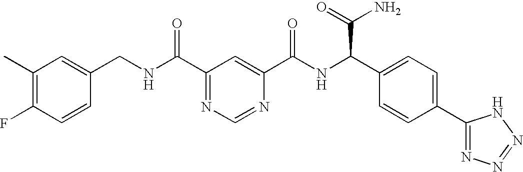 | <10 |
| 1004 | 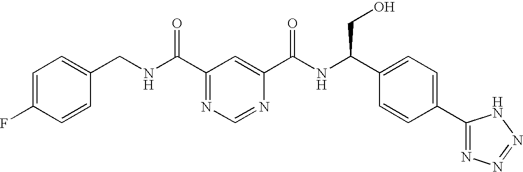 | <200 |

TABLE 2
Comparison of MMP-13 Activity Versus Location of Substituent (R² or R³) for Compounds
of Formula I.

| | Position of | | |
|---|---|---|---|
| | Methyl | | |
| | Substitution | | |
| Ex. # | (R² or R³) | Structure | IC₅₀ (nM) |
| 1006 | R³ | 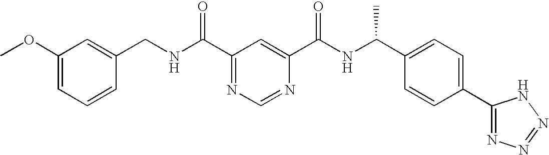 | >100 |
| 1005 | R² | 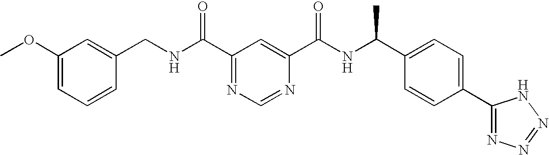 | <10 |
| 1040b | R² | 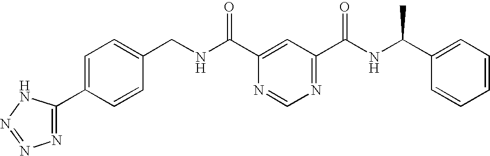 | <10 |
| 1040a | R³ | 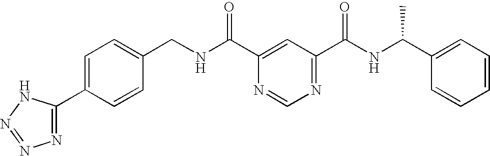 | >100 |

TABLE 3
Comparison of microsomal stability for R² Substituted versus Unsubstituted Compounds
of Formula I.

| Ex. # | Structure | Rat (%) | Human (%) |
|---|---|---|---|
| 1006 | 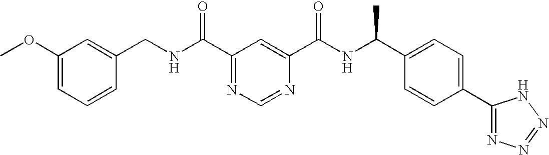 | 70 | 93 |
| 1040d | 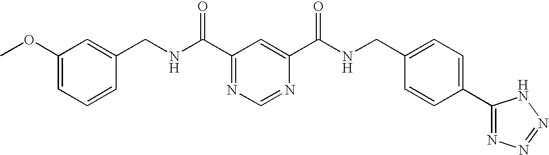 | 38 | 75 |
| 1040b | 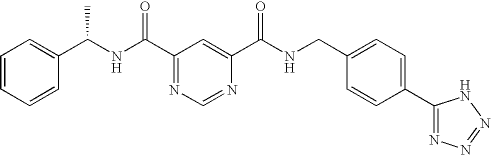 | 96 | 96 |
| 1007c | 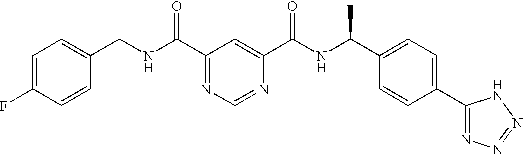 | 90 | 100 |
| 1040e | 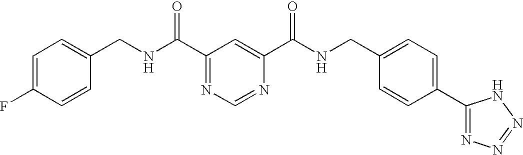 | 58 | 98 |

The synthesis of bis-amide metalloprotease inhibiting compounds of the invention and their biological activity assay are described in the following examples which are not intended to be limiting in any way.

### EXAMPLES AND METHODS

All reagents and solvents were obtained from commercial sources and used without further purification. Proton (¹H) spectra were recorded on a 400 MHz NMR spectrometer in deuterated solvents. Flash chromatography was performed using Merck silica gel, grade 60, 70-230 mesh using suitable organic solvents as indicated in specific examples. Thin layer chromatography (TLC) was carried out on silica gel plates with UV detection.

Preparative examples 1-205 are directed to intermediate compounds useful in preparing the compounds of the present invention.

In case the amines NH₂R¹ or NH₂CR²R³R⁴ are not commercially available, they can be synthesized in a similar way as described in the following section.

### Preparative Example 1

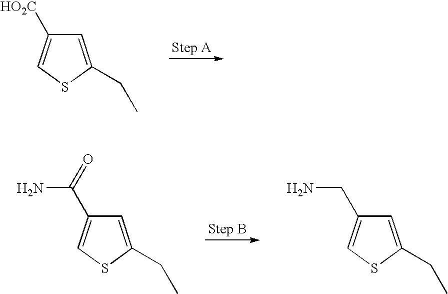 Step A

To commercially available 5-ethyl-thiophene-3-carboxylic acid (3.0 g) in dry methylene chloride (50 mL) at 0° C. was added oxalyl chloride (2.3 mL) followed by DMF (0.4 mL) and the mixture was stirred for 1 h at 0° C., then 3 h at room temperature. The reaction was then concentrated to an oil. The oil was then dissolved in methylene chloride (3 mL) and then slowly added to condensed ammonia (30 mL) at approx. −40° C. The reaction mixture was stirred at approx. −30° C. for 1 h and then allowed to slowly warm up to room temperature (˜10 h). The volatile components of the reaction mixture were removed under reduced pressure to give the intermediate (2.0 g; 68%) as a tan solid. [MH]⁺=156.

Step B

The intermediate from step A above (1.0 g) and tetrabutylammonium borohydride (4.9 g) in dry methylene chloride (30 mL) was vigorously stirred and heated (55-62° C.) for 24 h and then concentrated to an oil. To the chilled (0° C.) oil was slowly added 1N hydrochloric acid (15 mL) over a period of 1 h. The aqueous mixture was then heated at 100° C. for 1 h, cooled to room temperature, washed with diethyl ether (100 mL), basified with concentrated aqueous KOH to approx. pH 10. The aqueous phase was then extracted with diethyl ether (100 mL) and organic phase separated and dried (MgSO₄), filtered and concentrated to give the title compound (0.25 g; 27%) as an oil. [MH]⁺=142.

### Preparative Example 2

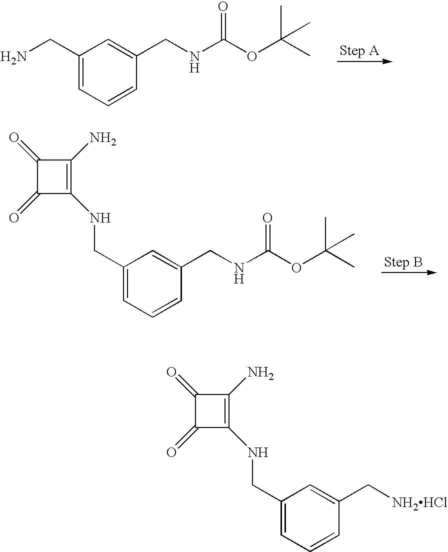 Step A

To a solution of 3,4-diethoxy-3-cyclobutene-1,2-dione (1.3 mL) in ethanol (40 mL) was added commercially available 1-(N-Boc-aminomethyl)-3-(aminomethyl)benzene (1.39 g). After 2 h ammonia (28% aqueous solution, 40 mL) was added and the mixture was stirred for additional 2 h and then evaporated under reduced pressure. The residue was slurried in methanol (20 mL) and filtered to give the intermediate (1.6 g; 82%).

Step B

A solution of the intermediate from step A above (400 mg) in hydrogen chloride (4M solution in dioxane) was stirred for 14 h, evaporated and dried to afford the title compound (317 mg; 98%) as an off-white solid. [M-Cl]⁺=232.

### Preparative Example 3

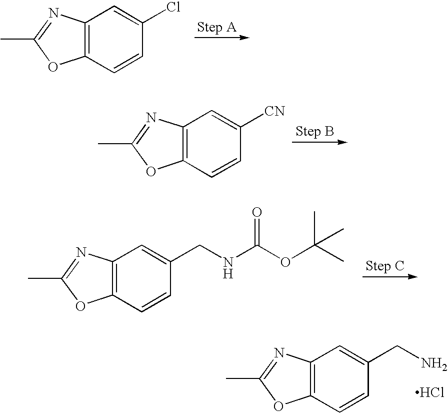 Step A

Commercially available 5-chloro-2-methylbenzoxazole (1.5 g), potassium cyanide (612 mg), dipiperidinomethane (720 μL), palladium diacetate (80 mg) and 1,5-bis-(diphenylphosphino)pentane (315 mg) were dissolved in dry toluene (20 mL), degassed and stirred at 160° C. in a sealed pressure tube under argon. After 24 h the mixture was diluted with ethyl acetate. The organic layer was washed with saturated ammonium chloride and brine, dried (MgSO₄), concentrated and purified by column chromatography (silica, cyclohexane/EtOAc, 9:1 to 7:3) to afford the intermediate (372 mg; 26%) as a colourless solid. ¹H-NMR (CDCl₃) δ=2.63 (s, 3H), 7.48-7.58 (s, 2H), 7.90 (s, 1H).

Step B

The intermediate from step A above (372 mg), di-tert-butyl dicarbonate (1.02 g) and nickel(II) chloride hexahydrate (56 mg) were dissolved in dry methanol (25 mL) and cooled to 0° C. Then sodium borohydride (400 mg) was added in portions and the ice bath removed. The mixture was vigorously stirred for 14 h, then diethylenetriamine (300 μL) was added and the mixture was concentrated to dryness. The residue was diluted with ethyl acetate, washed with 10% citric acid, saturated sodium hydrogen carbonate and brine, dried (MgSO₄), concentrated and purified by column chromatography (silica, cyclohexane/EtOAc, 7:3 to 6:4) to afford the intermediate (413 mg) as a colourless oil.

Step C

A solution of the intermediate from step B above (413 mg) in hydrogen chloride (4M solution in dioxane) was stirred for 2 h, diluted with diethyl ether and the precipitate was filtered, washed with diethyl ether to afford the title compound (341 mg; 73% over two steps) as a colourless solid. [M-Cl]=163.

### Preparative Example 4

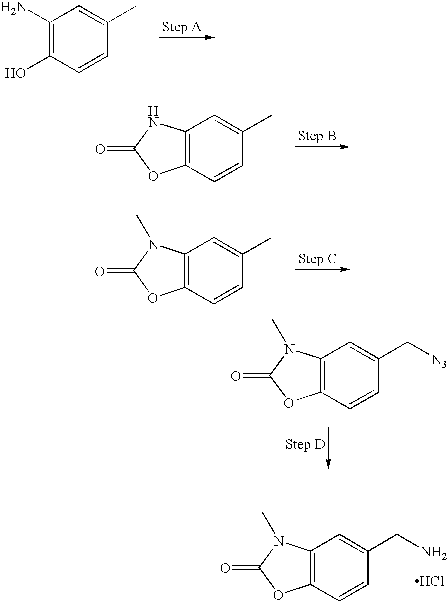 Step A

Commercially available 2-hydroxy-5-methylaniline (5.2 g) and N,N′-carbonyldiimidazole (6.85 g) were refluxed in dry THF (60 mL) for 6 h, cooled to room temperature, poured on ice and adjusted to pH 4 with 6N hydrochloric acid. The precipitate was filtered off, dried and recrystallized from toluene to afford the intermediate (4.09 g; 65%) as a grey solid.

Step B

The intermediate from step A above (1.5 g), potassium carbonate (1.7 g) and methyl iodide (6 mL) were dissolved in dry DMF (15 mL) and stirred at 50° C. for 2 h. The mixture was concentrated to dryness and acidified to pH 4 with 1N hydrochloric acid. The precipitate was filtered off and dried to afford the intermediate (1.48 g; 90%) as an off-white solid. ¹H-NMR (CDCl₃) δ=2.40 (s, 3H), 3.38 (s, 3H), 6.77 (s, 1H), 6.90 (d, 1H), 7.05 (s, 1H).

Step C

The intermediate from step B above (1.1 g), N-bromosuccinimide (1.45 g) and α,α′-azoisobutyronitrile (150 mg) were suspended in carbon tetrachloride (50 mL), degassed with argon and heated to reflux. After 1 h the mixture was cooled, filtered, evaporated and dissolved in dry DMF (20 mL). Then sodium azide (1 g) was added and the mixture was vigorously stirred for 3 h, diluted with ethyl acetate, washed with water and brine, dried (MgSO₄), concentrated and purified by column chromatography (silica, cyclohexane/EtOAc, 8:2 to 7:3) to afford the intermediate (963 mg; 70%) as colourless needles. ¹H-NMR (CDCl₃) δ=3.36 (s, 3H), 4.25 (s, 2H), 6.88 (s, 1H), 6.98 (d, 1H), 7.07 (s, 1H).

Step D

The intermediate from step C above (963 mg) and triphenylphosphine (1.36 g) in THF (30 mL) were stirred for 14 h, then water was added and the mixture was stirred for additional 2 h. The mixture was evaporated, coevaporated twice with toluene and diluted with dry dioxane. After addition of hydrogen chloride (4M solution in dioxane, 1.5 mL), the precipitate was filtered off and dried to afford the intermediate (529 mg; 52%) as a colourless solid. [M-Cl]⁺=179.

### Preparative Example 5

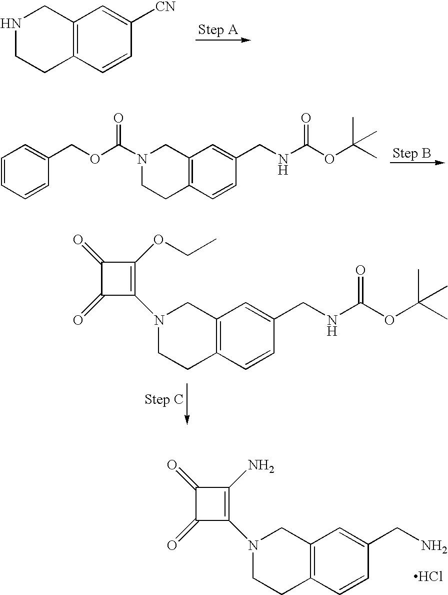 Step A

A solution of 7-cyano-1,2,3,4-tetrahydroisoquinoline (2.75 g), potassium carbonate (3.6 g) and benzylchloroformate (2.7 mL) in THF/water was stirred overnight and then evaporated under reduced pressure. The residue was diluted with ethyl acetate, washed subsequently with 10% citric acid, saturated sodium hydrogen carbonate and brine, dried (MgSO₄) and concentrated. The residue was dissolved in methanol (100 mL) and di-tert-butyl dicarbonate (7.6 g) and nickel(II) chloride hexahydrate (400 mg) was added. The solution was cooled to 0° C., then sodium borohydride (2.6 g) was added in portions. The mixture was allowed to reach room temperature and vigorously stirred overnight, then diethylenetriamine (2 mL) was added and the mixture was concentrated to dryness. The residue was diluted with ethyl acetate, washed with 10% citric acid, saturated sodium hydrogen carbonate and brine, dried (MgSO₄), concentrated and purified by column chromatography (silica, dichloromethane/methanol, 1:0 to 98:2) to afford the intermediate (1.81 g; 26%) as a colourless oil. [MH]⁺=397.

Step B

To a solution of intermediate from step A above (1.81 g) in ethanol (50 mL) was added palladium on charcoal (10 wt %, 200 mg) and then hydrogenated unter normal pressure overnight. The catalyst was filtered off and the solvent was evaporated to 20 mL. Then 3,4-diethoxy-3-cyclobutene-1,2-dione (0.68 mL) and trietylamine (0.5 mL) was added and the mixture was refluxed for 4 h. The solution was concentrated and purified by column chromatography (silica, cyclohexane/EtOAc, 6:4 to 1:1) to afford the intermediate (1.46 g; 83%) as a slowly crystallizing colourless oil.

Step C

To a solution of intermediate from step B above (1.46 g) in ethanol (20 mL) was added ammonia (28% aqueous solution, 100 mL) and the mixture was stirred for 3 h and then evaporated under reduced pressure. The residue was slurried in water, filtered and dried in vaccuo. To the residue was added hydrogen chloride (4M solution in dioxane, 20 mL) and stirred for 14 h, evaporated, suspended in diethyl ether, filtered and dried to afford the title compound (1.08 g; 92%) as an off-white solid. [M-Cl]⁺=258.

### Preparative Example 6

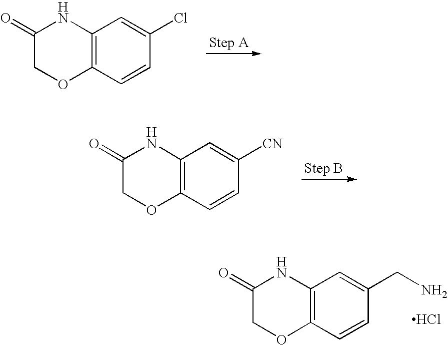 Step A

A solution of commercially available 6-chloro-4H-benzo[1,4]oxazin-3-one (3.2 g) and CuCN (2.9 g) in anhydrous N-methylpyrrolidone (15 mL) was stirred overnight in a pressure tube at 250° C. and then evaporated under reduced pressure. The residue was diluted with ethyl acetate, filtered and the remaining liquid was washed subsequently with 10% citric acid, saturated sodium hydrogen carbonate and brine, dried (MgSO₄) and concentrated. Crystallization from toluene/ethyl acetate afforded the intermediate (720 mg; 24%) as a tan solid. [MH]⁺=175.

Step B

The intermediate from step A above (377 mg), di-tert-butyl dicarbonate (1.3 g) and nickel(II) chloride hexahydrate (50 mg) were dissolved in dry methanol (30 mL) and cooled to 0° C. Then sodium borohydride (500 mg) was added in portions and the ice bath removed. The mixture was vigorously stirred for 6 h, then diethylenetriamine (300 μL) was added and the mixture was concentrated to dryness. The residue was diluted with ethyl acetate, washed with 10% citric acid, saturated sodium hydrogen carbonate and brine, dried (MgSO₄), concentrated and purified by column chromatography (silica, dichloromethane/methanol, 98:2) to afford the intermediate, which was stirred in hydrogen chloride (4M solution in dioxane; 12 mL) for 2 h and the evaporated to afford the title compound (214 mg; 41%) as a colourless solid. [M-Cl]⁺=179.

### Preparative Example 100

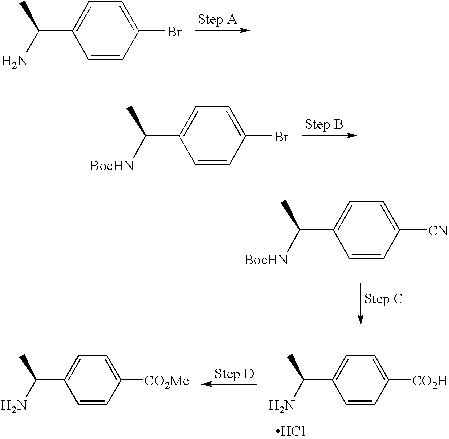 Step A

Commercially available (S)-(−)-1-(4-Bromophenyl)ethylamine (2.0 g) was dissolved in dry tetrahydrofuran (50 mL) and cooled to 0° C. and to this cooled solution was added di-t-butyl dicarbonate (2.0 g) dissolved in dichloromethane (3 μl) followed by Et₃N (2.8 mL). The solution was allowed to warm to room temperature. After stirring for 3 h, the mixture was concentrated and re-dissolved in dichloromethane (100 mL) This solution was washed with 1N HCl (2×50 mL) and saturated NaHCO₃ (50 mL). The organic layer was dried over anhydrous MgSO₄, filtered and concentrated to afford the intermediate (2.5 g; 92%) as a colourless solid. ¹H-NMR δ (CDCl₃) 1.35 (br s, 12H), 4.72 (br s, 2H), 7.17 (d, 2H), 7.43 (d, 2H).

Step B

The intermediate from step A above (4.0 g), ZnCN₂ (3.0 g) and Pd[PPh₃]₄ (1.5 g) were combined under nitrogen and anhydrous dimethylformamide (25 mL) was added. The yellow mixture was heated to 100° C. for 18 h and then concentrated under reduced pressure to afford crude compound which was purified by flash chromatography (20% hexane/dichloromethane) to give the title compound (2.0 g; 60%) as an oil. ¹H-NMR δ (CDCl₃) 0.89-1.62 (br m, 12H), 4.81 (br s, 2H), 7.42 (d, 2H), 7.65 (d, 2H). [MH]⁺=247.

Step C

The intermediate from step B above (2.0 g) was suspended in 6N HCl (50 mL) and heated to 100-105° C. for 20 hours upon which the solution becomes homogeneous. The solvent was removed under reduce pressure to give the intermediate (1.8 g; quantitative) as a colourless solid.

Step D

The intermediate from step C above (1.0 g) was dissolved in anhydrous MeOH (150 mL) saturated with anhydrous HCl gas. The reaction mixture was then heated to reflux for 20 hours. After cooling to room temperature, the solvent was removed under reduced pressure to give a solid. The solid was taken up in CH₂Cl₂ and washed with saturated NaHCO₃. The organic was separated and dried over MgSO₄, filtered and concentrated to give the title compound (0.31 g; 35%) as an oil which slowly crystallized into a light brown solid. [MH]⁺=180.

### Preparative Example 101

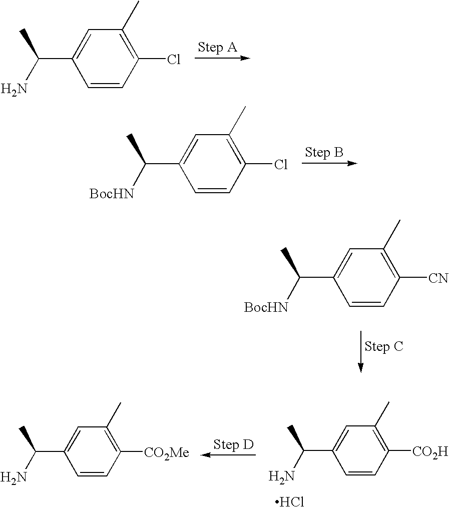 Step A

Commercially available (S)—(4-chloro-3-methylophenyl)ethylamine (1.5 mmol) was dissolved in dry tetrahydrofuran (10 mL) and cooled to 0° C. and to this cooled solution was added di-t-butyl dicarbonate (1.5 mmol) dissolved in of CH₂Cl₂ (1.0 mL) followed by Et₃N (2.8 mL). The solution was allowed to warm to room temperature. After stirring for 3 hours, the mixture was concentrated and re-dissolved in CH₂Cl₂ (100 mL). This solution was washed with 1N HCl (2×50 mL) and saturated NaHCO₃ (50 mL). The CH₂Cl₂ layer was dried over anhydrous MgSO₄, filtered, and concentrated to afford the title compound.

Step B

If one were to add to the Boc protected amine product (1 mmol) ZnCN₂ (2 mmol), Pd[PPh₃]₄ (0.1 mmol) and anhydrous dimethylformamide (6 mL) and heat the yellow mixture to 100° C. for 18 h and then purified by flash chromatography (20% hexane/CH2Cl2) one would obtain the desired cyano containing compound.

Step C

If one were to suspend the cyano containing compound (0.5 mmol) in 6N HCl (10 mL) and heat to 100-105° C. for 20 h until the solution becomes homogeneous and then remove the solvent under reduce pressure one would obtain the amino acid as the hydrochloride salt.

Step D

If one were to dissolve the hydrochloride salt of the amino acid (0.5 mmol) in anhydrous MeOH (50 mL) and then saturate with anhydrous HCl gas and then heat to reflux for 20 hours one would obtain the 4-(1(S)-amino-ethyl)-2-methyl-benzoic acid methyl ester.

### Preparative Example 102

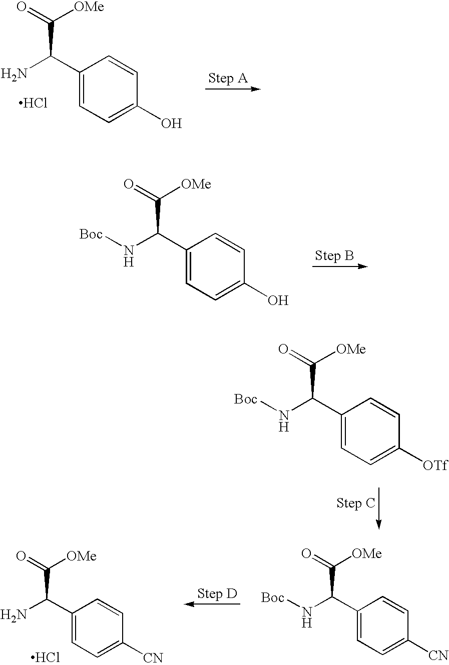 Step A

Commercially available (R)-methyl 2-amino-2-(4-hydroxyphenyl)acetate hydrochloride (3.57 g), t-butyl dicarbonate (4.735 g) and triethylamine (6.87 mL) were added to THF (40 mL) and stirred at room temperature. After 15 h the mixture was diluted with H₂O (50 mL) and extracted with ethyl acetate. The organic layer was dried over MgSO4, concentrated and purified by column chromatography (silica, hexane/EtOAc) to afford the title compound (2.77 g; 95%) as a colourless solid. [MNa]⁺=304.

Step B

The intermediate from step A above (1.557 g) and pyridine (1.12 mL) were added to CH₂Cl₂ (50 mL). After the solution was cooled to −78° C., triflate anhydride (1.03 mL) was added dropwise to the solution. The reaction mixture was stirred for 12 h while gradually warm up to room temperature. The mixture was concentrated under reduced pressure and purified by column chromatography (silica, hexane/EtOAc) to afford the title compound (2.29 g; 100%). [MNa]⁺=436.

Step C

To the solution of the intermediate from step B above (4.025 g) in DMF (25 mL) were added Pd₂(dba)₃ (72 mg) and dppf (174 mg). The mixture was heated up to 110° C. and zinc cyanide (1.372 g) was added. After stirred for 1 day, the mixture was concentrated under reduced pressure and purified by column chromatography (silica, hexane/EtOAc) to afford the title compound (2.206 g; 78%). [MNa]⁺=313.

Step D

The intermediate from step C above (1.375 g) was added to HCl solution (4N in dioxane, 3 mL). After 12 h, hexane (30 mL) was added and the colourless solid was collected through filtration to afford the title compound (1.047 g; 97%). [MH]⁺=191.

### Preparative Example 103

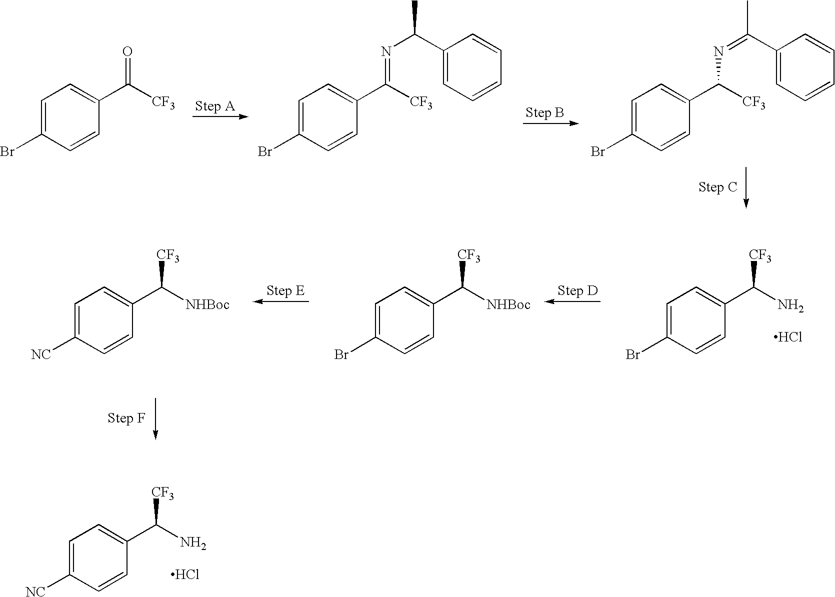 Step A

To the mixture of commercially available 4-bromo trifluoroacetophenone (2 g) and (S)-phenyl ethylamine (0.98 g) in toluene (20 mL) was added titanium chloride (0.5 mL) in toluene (4 mL) and was stirred for 1 h at room temperature. The resulting salt was filtered, and the filtrate was concentrated. The crude mixture was run through a short silca gel column to give the title compound (1.8 g).

Step B

To the intermediate from step A above was added DBU (0.35 mL). The solution was stirred for 4 h. The mixture was loaded directly on a short silca gel column and rinsed with hexane to give the title compound (1.7 g).

Step C

To the intermediate from step B above was added hydrogen chloride in diethyl ether (10 mL, 2N). The reaction was stirred for 1 h and the resulting precipitate was collected by filtration and rinsed with diethyl ether (5 mL) to give the title compound (0.88 g).

Step D

To the intermediate from step C above (0.88 g) in dichloromethane (10 mL) was added di-t-butylcarbonate and triethylamine at 0° C. The reaction was stirred for 3 h. The solution was washed with hydrochloric acid (3 mL, 1N), saturated brine (2 mL) and dried over sodium sulfate, filtered and volatile components removed under reduced pressure to give give the title compound.

Step E

The intermediate from step D above, zinc cyanide (706 mg), palladium tetrakis triphenylphosphine (330 mg) in anhydrous dimethylforamide (5 mL) was heated to 100° C. overnight. The reaction mixture was concentrated to dryness and purified by silica gel chromatography to give the title compound.

Step F

To the intermediate from step E above was added hydrogen chloride in diethyl ether (10 mL, 2N). The reaction was stirred for 1 h and the resulting precipitate was collected by filtration and rinsed with diethyl ether (5 mL) to give the title compound (0.85 g; 75%).

### Preparative Example 104

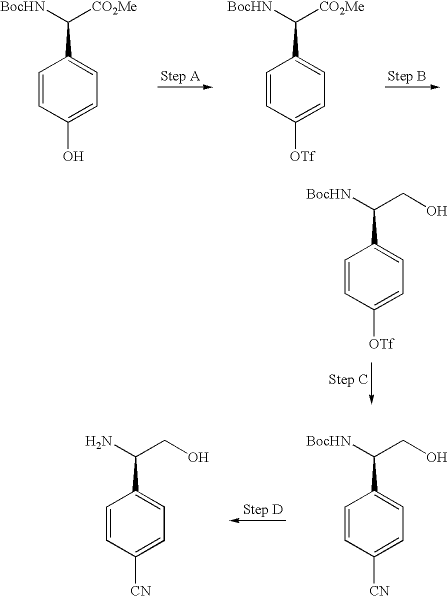 Step A

At 0° C., triflic anhydride (0.6 mL) was added to N-Boc-4-hydroxyphenyl glycine (0.92 g) and pyridine (0.43 mL) in dichloromethane (10 mL). The reaction was kept at the same temperature for 2 h, and hydrochloric acid (3 mL, 1N) was added. The organic layer was separated and washed with brine (2 mL), dried over magnesium sulfate and concentrated to give the title compound.

Step B

At 0° C., to intermediate from step A above in methanol (10 mL) was added sodium borohydride powder in portions (500 mg). The reaction was stirred for 30 min and hydrochloric acid (3 mL, 1N) was added to quench the reaction. The solution was concentrated to get rid of methanol. The mixture was extracted with ethyl acetate (3×5 mL) and then the combined organic layer was washed with brine (3 mL), dried over magnesium sulfate and concentrated to give the title compound (578 mg; 46% for two steps).

Step C

To a mixture of the intermediate from step B above, zinc cyanide (353 mg), palladium dibenzoaacetone (28 mg), bis(diphenylphosphino)ferecene (65 mg) in anhydrous N,N-dimethylforamide (5 mL) was heated to 100° C. for 3 h. The reaction mixture was concentrated to dryness, and purified by silca gel chromatography to give the title compound.

Step D

To the intermediate from step C above was added anhydrous hydrochloric acid (5 mL, 4N in dioxane) and the reaction was stirred for 1 h at room temperature. The colourless solid that was formed was collected and rinsed with diethyl ether to give the title compound (246 mg; quantitative for 2 steps).

### Preparative Example 200

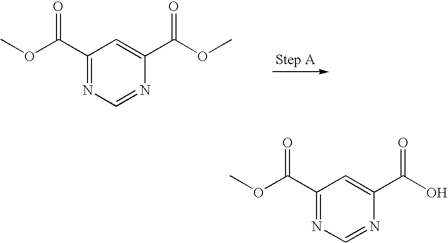 Step A

To a solution of sodium hydroxide (1.00 g) in dry methanol (50 mL) was added commercially available pyrimidine-4,6-dicarboxylic acid dimethyl ester (4.91 g). The resulting suspension was stirred at room temperature for 1 h. Then a 4M solution of hydrochloric acid in dioxane (6.25 mL) was added and stirring at room temperature was continued for 10 min. The mixture was concentrated and purified by flash chromatography (silica, dichloromethane/methanol) to afford the title compound (3.48 g; 76%). [MH]⁺=183.

### Preparative Example 201

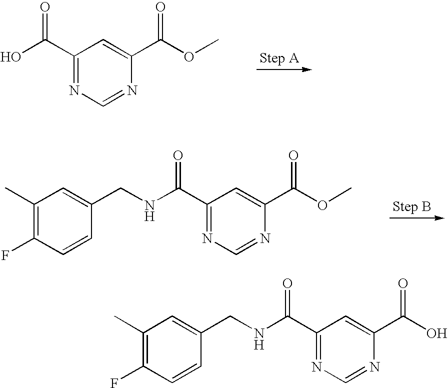 Step A

To a solution of the title compound from the Preparative Example 200 (2.29 g) and N-methylmorpholine (3.32 mL) in dry THF (250 mL) was added ethyl chloroformate (1.19 mL) at −30° C. After 1 h at this temperature 4-fluoro-3-methylbenzylamine (1.75 g) was added and the resulting mixture was stirred for 16 h allowing the temperature to raise from −30° C. to 10° C. The mixture was concentrated and absorbed on silica. Purification by column chromatography (silica, cyclohexanelethyl acetate) afforded the title compound (2.39 g; 62%) as a colourless solid. [MH]⁺=304.

Step B

To a solution of the title compound of step A above (2.39 g) in tetrahydrofuran (50 mL) and water (50 mL) was added a lithium hydroxide (496 mg) at room temperature. After 2 h at room temperature the mixture was acidified with 1M hydrochloric acid to pH 2. The aqueous layer was extracted with ethyl acetate twice and the combined organic layers were dried (MgSO₄) and concentrated to afford the title compound (2.23 g; 97%) as a colourless solid. [MH]⁺=290.

### Preparative Example 202

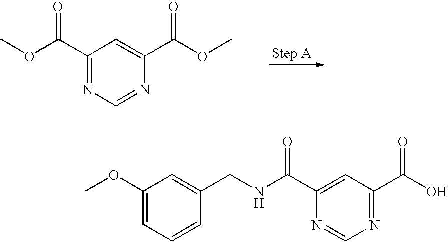 Step A

A solution of commercially available pyrimidine-4,6-dicarboxylic acid dimethyl ester (1.96 g) and commercially available 3-methoxy-benzylamine (1.38 mL) in dry N,N-dimethylformamide (10 mL) was placed in a preheated oil bath (˜80° C.). After stirring at this temperature for 18 h the mixture was concentrated and flash filtered (silica, cyclohexane/ethyl acetate). The obtained material was suspended in dry tetrahydrofuran (10 mL) and treated with a solution of lithium hydroxide (642 mg) in water (15 mL). The resulting mixture was stirred at room temperature for 16½ h, diluted with water (35 mL), washed with dichloromethane (3×50 mL) and acidified by addition of a 1M aqueous solution of hydrochloric acid (20 mL). The formed precipitate was isolated by suction, washed with water (2×50 mL) again suspended/dissolved in water (200 mL) and ultrasonificated for 5 min. The remaining precipitate was isolated by suction and dried under reduced pressure to afford the title compound (700 mg; 24%). [MH]⁺288.

### Preparative Example 203

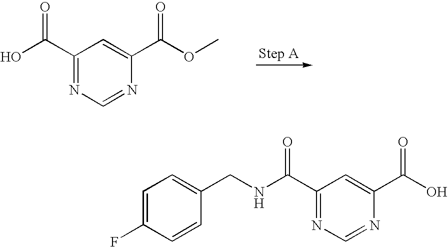

Following a similar procedure as that described in Preparative Example 201, except using 4-fluorobenzylamine as amine, the title compound was prepared. [MH]⁺276.

### Preparative Example 204

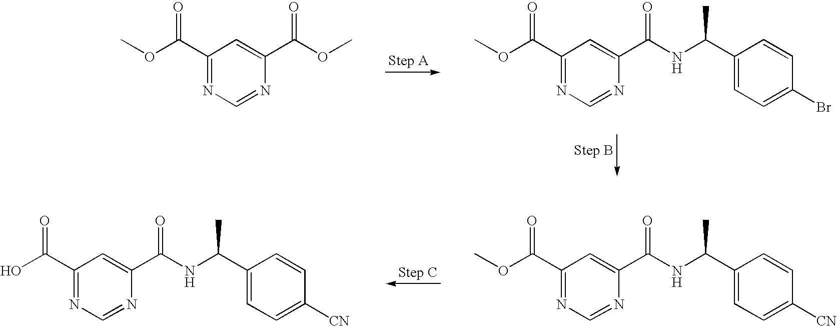 Step A

A solution of commercially available pyrimidine-4,6-dicarboxylic acid dimethyl ester (7.14 g) and commercially available (S)-1-(4-bromophenyl)ethylamine (5.06 g) in dry N,N-dimethylformamide (30 mL) was heated to 70° C. for 3 d. The solution was diluted with ethyl acetate and washed with 1N HCl, water and brine. Purification by flash filtered (silica, cyclohexane/ethyl acetate 7:3) afforded the intermediate (5.65 g; 61%) as a colourless oil. [MH]⁺=364/366.

Step B

The intermediate from step A above (5.65 g), zinc cyanide (1.37 g), palladium tetrakis triphenylphosphine (451 mg) in anhydrous dimethylforamide (5 mL) was degassed under Argon and heated to 80° C. overnight. The reaction mixture was concentrated to dryness, diluted with ethyl acetate and washed with 1N HCl, water and brine. Purification by flash filtered (silica, cyclohexane/ethyl acetate 6:4 to 4:6) afforded the intermediate (3.99 g; 82%) as colourless crystals. [MH]⁺=311.

Step C

To a solution of the title compound of step B above (2.77 g) in tetrahydrofuran (50 mL) was added LiOH.H₂O (560 mg) at room temperature. After 2 h at room temperature the mixture was acidified with 1M hydrochloric acid to pH3. The aqueous layer was extracted with ethyl acetate twice and the combined organic layers were dried (MgSO₄) and concentrated to afford the title compound (2.75 g; quantitative) as a off-white solid. [MH]⁺=297.

### Preparative Example 205

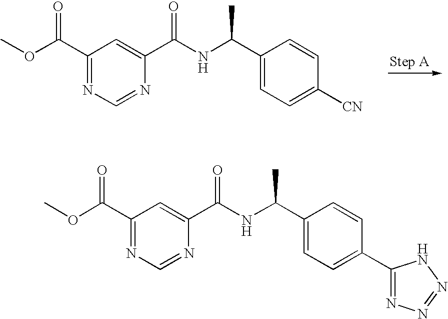 Step A

To a solution of the title compound from Preparative Example 204, step B (308 mg) in dry toluene (2 mL) were added TMSN₃ (200 μL) and dibutyltin oxide (30 mg). The mixture was heated up to 100° C. and stirred overnight. After cooling to room temperature, filtration and drying at high vacuum afforded the title compound (256 mg; 73%). [MH]⁺=354.

### Example 999

### Assay for Determining MMP-13 Inhibition

The typical assay for MMP-13 activity is carried out in assay buffer comprised of 50 mM Tris, pH 7.5, 150 mM NaCl, 5 mM CaCl₂ and 0.05% Brij-35. Different concentrations of tested compounds are prepared in assay buffer in 50 μL aliquots. 10 μL of 40 nM stock solution of MMP-13 enzyme is added to the compound solution. The mixture of enzyme and compound in assay buffer is thoroughly mixed and incubated for 20 minutes at room temperature. Upon the completion of incubation, the assay is started by addition of 40 μL of 12.5 μM stock solution of MMP-13 fluorogenic substrate (Calbiochem Cat. No. 444235). The time-dependent increase in fluorescence is measured at the 325 nm excitation and 393 nm emission by automatic plate multireader. The IC₅₀ values are calculated from the initial reaction rates. Inhibition activity of highly potent compounds of Formula I are summarized in Table 1. Selectivity assays were run in a similar manner using MMP-1, MMP-3, MMP-8, MMP-12, MMP-14 and TACE.

### Example 999a

### Assay for Microsomal Stability

For microsomal stability testing 1 μM conzentration of compound and human or rat microsomes (0.3 mg/mL, BD bioscience) are used in the in vitro assay. To ensure proper energy supply for microsomal degradation of compound, an energy regenerating system comprised of NADP, glucose 6-phosphate and glucose 6-phosphate dehydrogenase is added to samples and suspension is incubated for 60 min at 37° C. in rotary shaker. After incubation time, acetonitrile containing internal standard is added to stop metabolization by precipitation of proteins. After centrifugation step, supernatant is analysed by LC-MS/MS and percentage of compound remaining is analysed.

### Example 1000

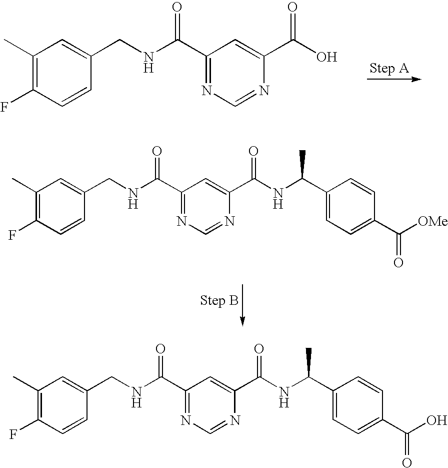 Step A

To a solution of the title compound from Preparative Example 201 (0.5 g) and N-methylmorpholine (0.21 mL) in dry THF (6 mL) was added isobutyl chloroformate (0.25 mL) at −30° C. After 1 h at this temperature the title compound from Preparative Example 100 (0.31 g) was added and the resulting mixture was stirred for 16 h allowing the temperature to raise from −30° C. to 10° C. The mixture was concentrated and absorbed on silica. Purification by column chromatography (silica, methylene chloride/diethylether) afforded the title compound (0.45 g; 57%) as a light yellow foam. [MH]⁺=451.

Step B

To a solution of the intermediate from step A above (0.4 g) in tetrahydrofuran (3 mL) was added 3 mL of 1M lithium hydroxide solution at room temperature and allowed to stir for 12 hours. The mixture was acidified with 1N hydrochloric acid to pH 2. The solid was filtered and washed with water and then ether and then dried to give the title compound (0.3 g; 78%) as a colourless solid. [MH]⁺=437.

### Examples 1000a-1000f

If one were to follow a similar procedure as that described in Preparative Example 202 using the pyrimidine core unit and amine A to give the resulting acid B and then couple amine from Preparative Example 100 as described in Example 1000, one would obtain compounds as indicated in the table below.

| Ex. # | Amine A | Pyrimidine Acid B |
|---|---|---|
| 1000a | 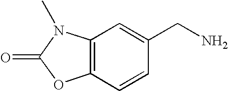 | 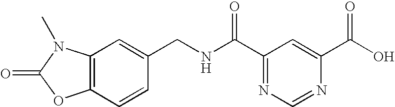 |
| 1000b | 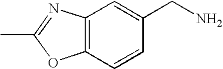 | 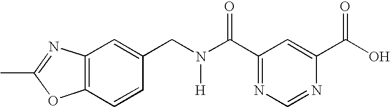 |
| 1000c | 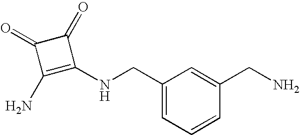 | 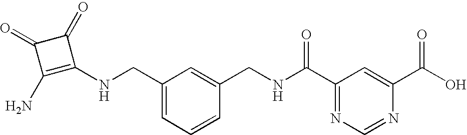 |
| 1000d | 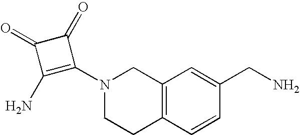 | 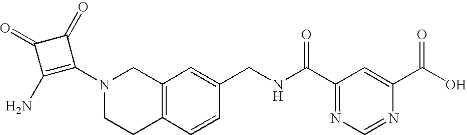 |
| 1000e | 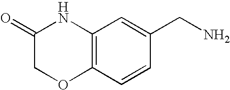 | 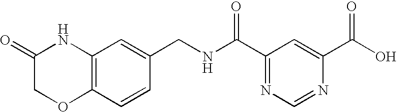 |
| 1000f | 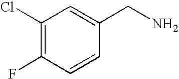 | 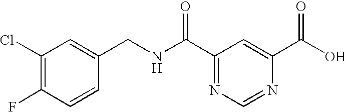 |

| Ex. # | CompoundExamples |
|---|---|
| 1000a | 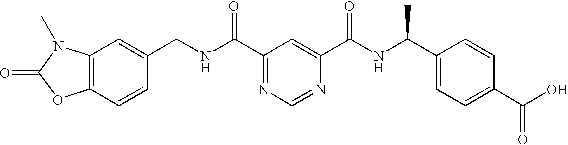 |
| 1000b | 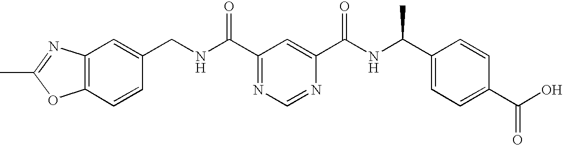 |
| 1000c | 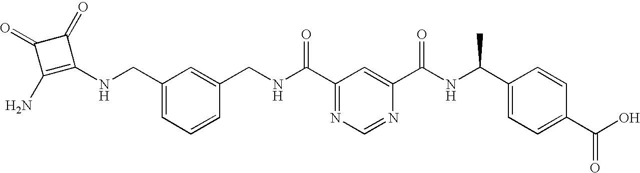 |
| 1000d | 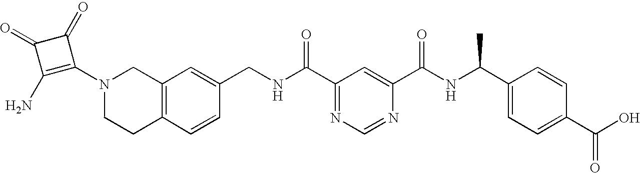 |
| 1000e | 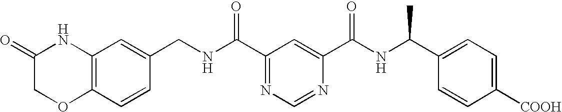 |
| 1000f | 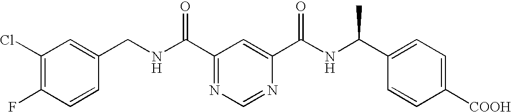 |

### Example 1001

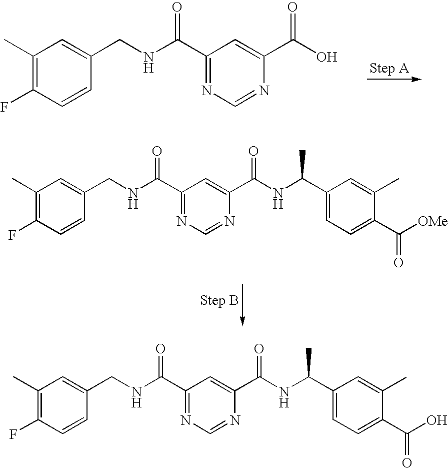 Step A

If one were to add to a solution of the title compound from Preparative Example 201 (0.5 g) and N-methylmorpholine (0.21 mL) in dry THF (6 mL) isobutyl chloroformate (0.25 mL) at −30° C. and then after 1 h at this temperature add (S)-4-(1-Amino-ethyl)-2-methyl-benzoic acid methyl ester (Preparative Example 101) and then stir the resulting mixture for 16 h allowing the temperature to rise from −30° C. to 10° C., then concentrate the mixture and purify the resulting crude material by column chromatography one would afford the title compound.

Step B

If one were to add to a solution of the intermediate from Step A above (0.25 g) in tetrahydrofuran (2 mL) a slight excess of 1M lithium hydroxide solution at room temperature and allow to stir for 12 h and then acidify the mixture with 1N hydrochloric acid to pH 2 and then filter the solid and wash the solid with water one would afford after further washing with diethylether the title compound.

### Examples 1001a-1001n

If one were to follow a similar procedure as that described in Preparative Example 202 using the pyrimidine core unit and amine A to give the resulting acid B and then couple amine C as described in Example 1001, one would obtain compounds as indicated in the table below.

| Ex. # | Amine A | Acid B |
|---|---|---|
| 1001a | 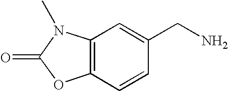 | 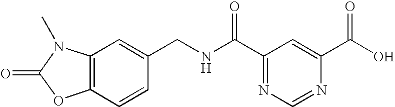 |
| 1001b | 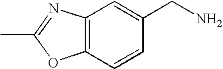 | 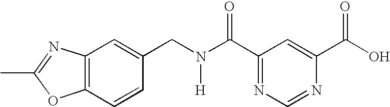 |
| 1001c | 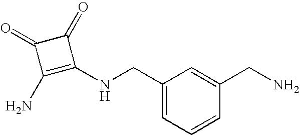 | 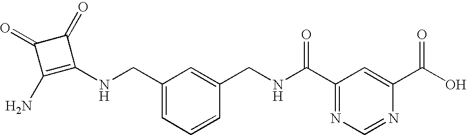 |
| 1001d | 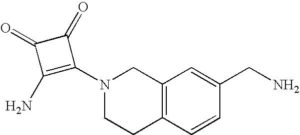 | 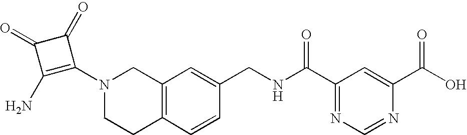 |
| 1001e | 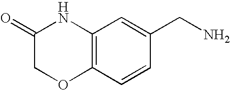 | 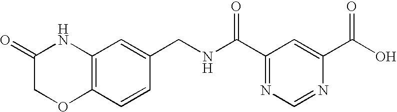 |
| 1001f | 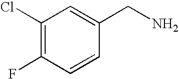 | 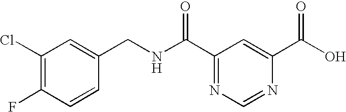 |
| 1001g | 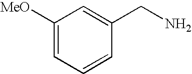 | 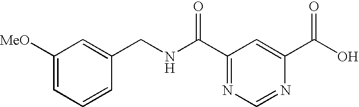 |
| 1001h | 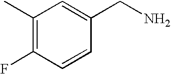 | 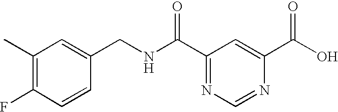 |
| 1001i | 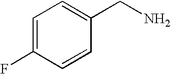 | 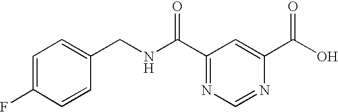 |
| 1001j | 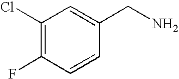 | 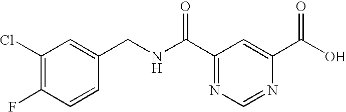 |
| 1001k | 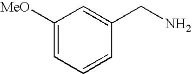 | 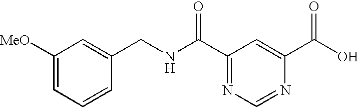 |
| 1001l | 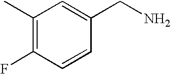 | 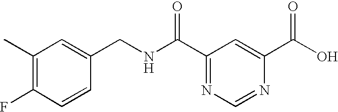 |
| 1001m | 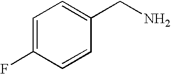 | 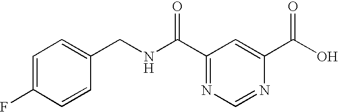 |
| 1001n | 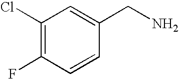 | 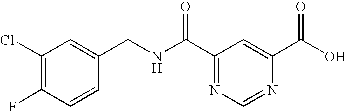 |

| Ex. # | Amine C | CompoundExamples |
|---|---|---|
| 1001a | 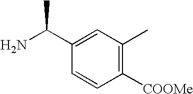 | 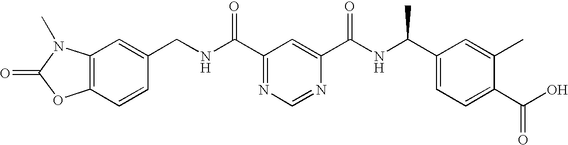 |
| 1001b | 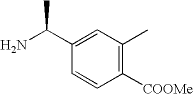 | 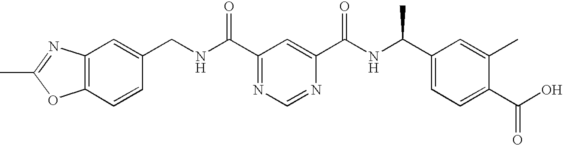 |
| 1001c | 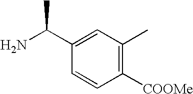 | 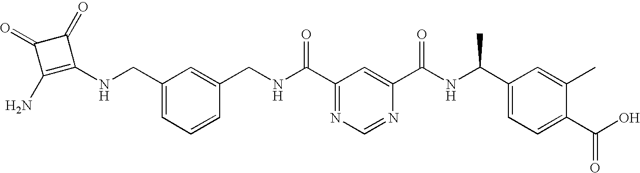 |
| 1001d | 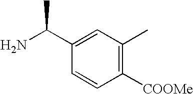 | 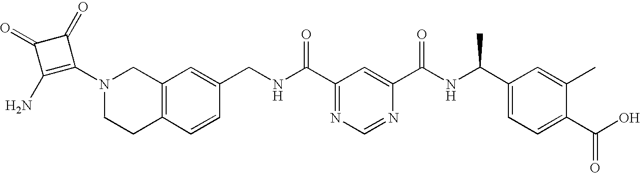 |
| 1001e | 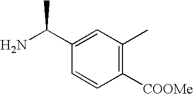 | 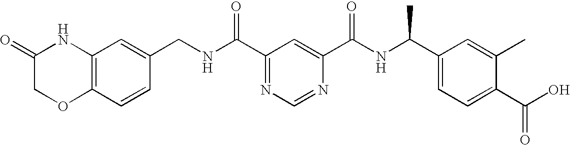 |
| 1001f | 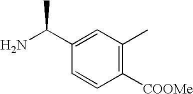 | 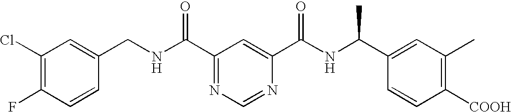 |
| 1001g | 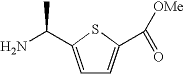 | 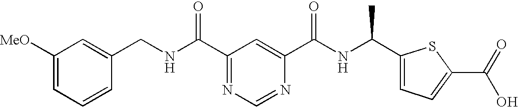 |
| 1001h | 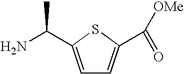 | 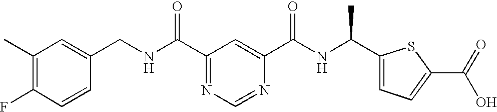 |
| 1001i | 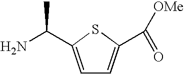 | 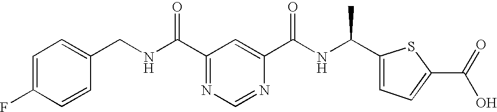 |
| 1001j | 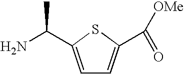 | 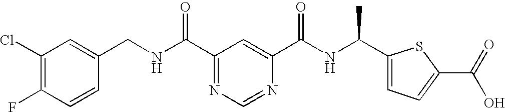 |
| 1001k | 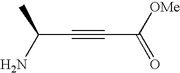 | 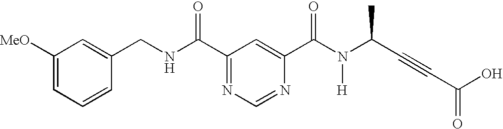 |
| 1001l | 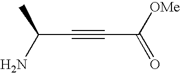 | 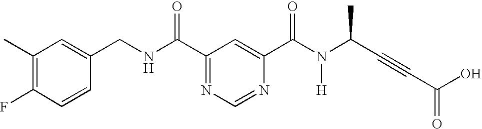 |
| 1001m | 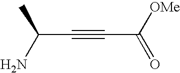 | 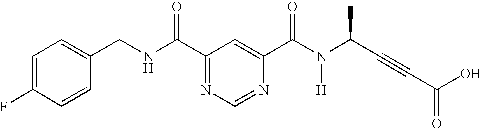 |
| 1001n | 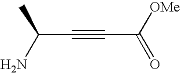 | 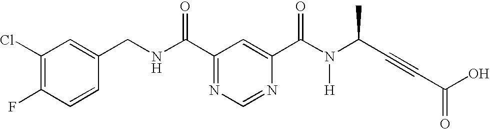 |

### Example 1002

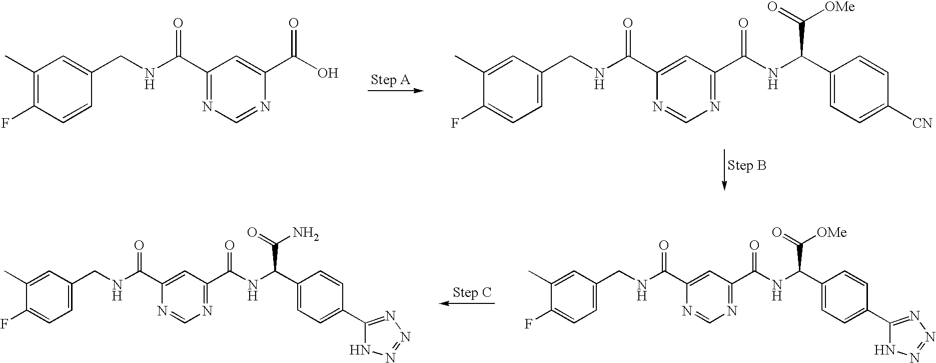 Step A

To a solution of the title compound from Preparative Example 201 (300 mg) in THF (30 mL) was added the title compound from Preparative Example 102 (258 mg), EDCI (298 mg), HOBt (154 mg) and K₂CO₃ (665 mg). The solution was stirred for 12 h and diluted with EtOAc. The mixture was washed with aqueous NaHCO₃, aqueous NH₄Cl and brine. The organic layer was dried over MgSO4, concentrated and purified by column chromatography (silica, hexane/EtOAc) to afford the title compound (469.6 mg; 98%) as a colourless solid. [MH]⁺=462.5.

Step B

To a solution of the intermediate from step A above (104 mg) in dioxane (2 mL) were added TMSN₃ (129 mg) and dibutyltin oxide (11 mg). The mixture was heated up to 80° C. and stirred for 12 h. The mixture was concentrated under reduced pressure and purified by column chromatography (CH₂Cl₂/MeOH) to afford the title compound (109 mg; 99%). [MH]⁺=505.5.

Step C

The intermediate from step B above (13.3 mg) was added to ammonia in MeOH (7N). The solution was stirred for 12 h and concentrated down to afford the title compound (13.0 mg). [MH]⁺=490.3.

### Examples 1002a-1002e

If one were to follow a similar procedure as that described in Preparative Example 202 using the pyrimidine core unit and amine A to give the resulting acid B and then couple the amine from Preparative Example 102 as described in Example 1002, one would obtain compounds as indicated in the table below.

| Ex. # | Amine A | Pyrimidine Acid B |
|---|---|---|
| 1002a | 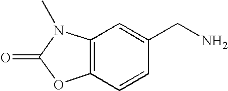 | 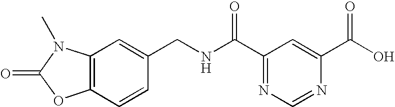 |
| 1002b | 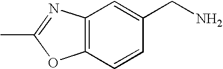 | 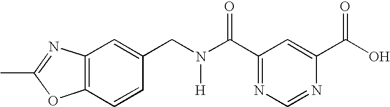 |
| 1002c | 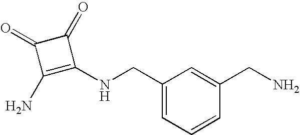 | 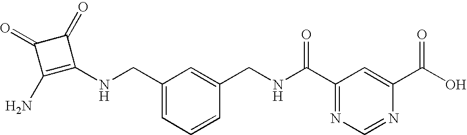 |
| 1002d | 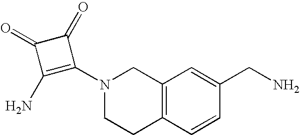 | 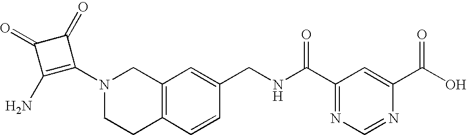 |
| 1002e | 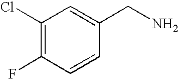 | 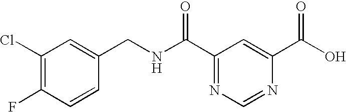 |

| Ex. # | CompoundExamples |
|---|---|
| 1002a | 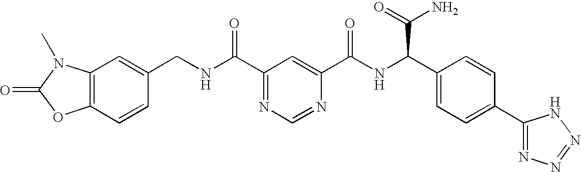 |
| 1002b | 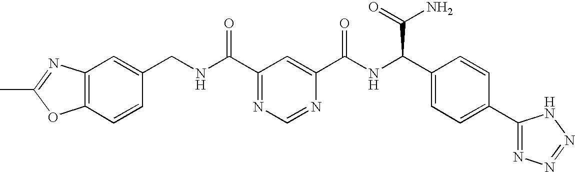 |
| 1002c | 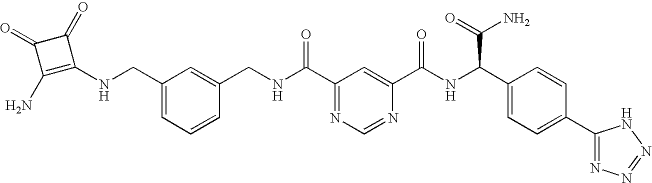 |
| 1002d | 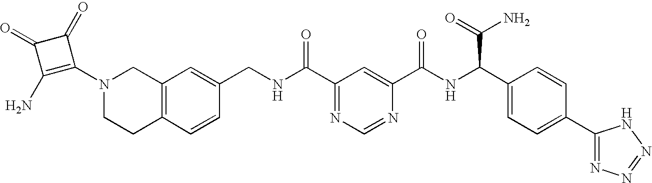 |
| 1002e | 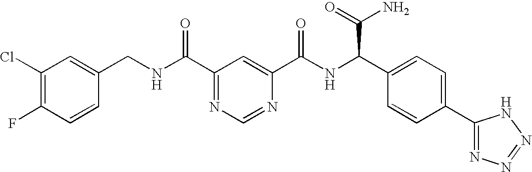 |

### Example 1003

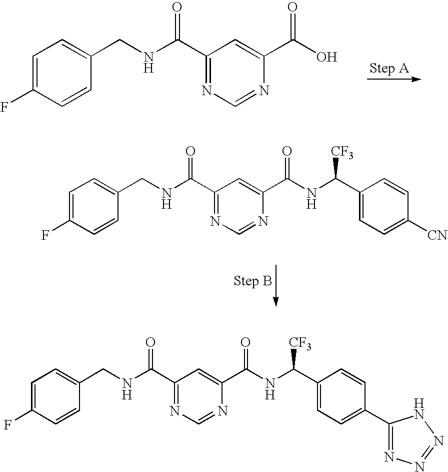 Step A

To the title compound from Preparative Example 202 (323 mg) the title compound from Preparative Example 103 (237 mg), triethylamine (0.35 mL) in THF (5 mL) was added PyBop (550 mg) at room temperature. The reaction mixture was stirred for 1 h and then concentrated to dryness. The solid was dissolved in ethyl acetate (20 mL) and the resulting solution was washed with hydrochloric acid (5 mL, 1M), saturated sodium bicarbonate (5 mL) and brine (5 mL). The solution was dried over magnesium sulfate and concentrated in vaccuo. The crude mixture was purified by silica gel chromatography to give the intermediate (300 mg; 65%). [MH]⁺=458.

Step B

To the intermediate from step A above (57 mg) and azidotrimethylsilane (34 μL) in toluene (10 mL) was added dibutyltin oxide (3.1 mg). The suspension was heated to reflux overnight and then concentrated to dryness. The product was washed with dichloromethane (2×1 mL) to give the title compound (30 mg; 48%). [MH]⁺=501.

### Example 1004

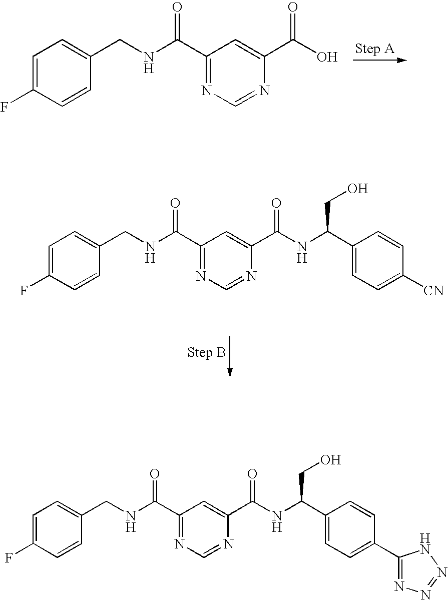 Step A

To the title compound from Preparative Example 202 (95 mg) and 4-methylmorpholine (40 μL) in THF (3 mL) was added isobutylchloroformate (47 μL) at −30° C. The reaction is allowed to warm to −10° C. in 0.5 h and then cooled to −30° C. A solution of the title compound from Preparative Example 104 and 4-methylmorpholine (40 μL) in N,N-dimethylformamide (1 mL) was added dropwise. The mixture was stirred overnight and allowed to warm to room temperature. The solution was concentrated to dryness and purified by silica gel chromatography (dichloromethane/methanol 50:1 to 10:1) to give the intermediate (85 mg; 59%). [MH]⁺=420.

Step B

To the mixture of the intermediate from step A above (68.5 mg) and azidotrimethylsilane (90 μL) in toluene (2 mL) was added dibutyltin oxide (8.1 mg). The suspension was heated to reflux overnight and then concentrated to dryness. The product was washed with dichloromethane (2×1 mL) to give the title compound (52 mg; 69%). [MH]⁺=463.

### Example 1005

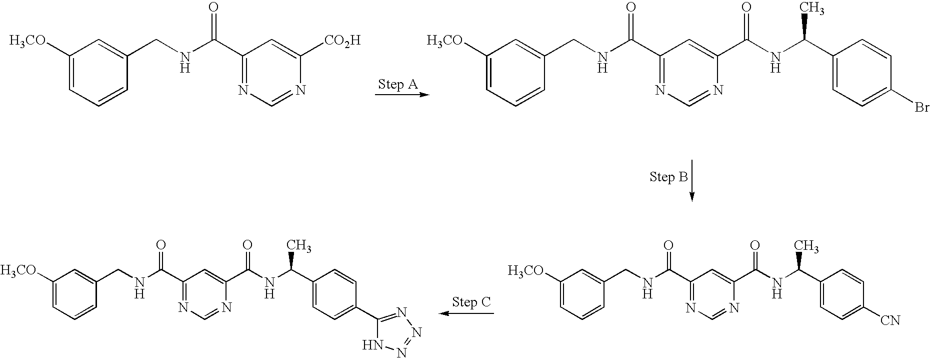 Step A

The title compound from Preparative Example 203 (100 mg) was dissolved in a mixture of anhydrous THF (0.5 mL) and anhydrous DMF (0.5 mL) under nitrogen and the reaction vessel was cooled to −20° C. To this cooled solution was added N-methylmorpholine (38 μL) followed by isobutylchloroformate (46 μL) and the cooled mixture was stirred for an additional 1 h upon which a solution of commercially available (S)-1-(4-bromophenyl)ethylamine (56 mg) in THF (1 mL) was added. The mixture was stirred for 2 h at −20° C. and gradually warmed to room temperature and stirred for 8 h. The reaction mixture was concentrated under reduced pressure and the crude material was chromatographed (dichlormethane/methanol 97:3) to give the intermediate (137 mg; 95%). ¹H NMR δ (CDCl₃) 1.60 (d, 3H), 3.70 (s, 3H), 4.50 (d, 2H), 5.30 (m, 1H), 6.70-6.90 (m, 4H), 7.60 (d, 2H), 8.00 (d, 2H), 8.40 (s, 1H), 9.40 (s, 1H), 9.50 (d, 1H), 9.60 (t, 1H).

Step B

The intermediate from step A above (146 mg) was combined with Zn(II) cyanide (70 mg) and Pd(PPh₃)₄ (35 mg) under nitrogen and to this mixture was added dry DMF (2 mL). This mixture was then heated to 100° C. for 10 hours. After cooling to room temperature, the volatiles were removed under high vacuum and the remaining residue was chromatographed (dichlormethane/methanol 97:3) to give of the intermediate (114 mg; 90%). ¹H NMR δ (CDCl₃) 1.65 (d, 3H), 3.60 (s, 3H), 4.50 (d, 2H), 5.35 (m, 1H), 6.75-6.95 (m, 4H), 7.65 (d, 2H), 8.10 (d, 2H), 8.45 (s, 1H), 9.35 (s, 1H), 9.50 (d, 1H), 9.60 (t, 1H).

Step C

The intermediate from step B above (137 mg) and Bu₂SnO (10 mg) were suspended in anhydrous toluene (3 mL) upon which TMSN₃ (88 μL) was added. The mixture was then heated to 110° C. for 10 hours. After cooling to room temperature, the toluene was removed under high vacuum and the remaining residue was chromatographed (dichlormethane/methanol 80:20) to give the title compound (95 mg; 63%) as a colourless solid. ¹H NMR δ (CDCl₃) 1.60 (d, 3H), 3.70 (s, 3H), 4.45 (d, 2H), 5.30 (m, 1H), 6.80 (d, 1H), 6.90(m, 2H), 7.20 (t, 1H), 7.60 (d, 2H), 8.00 (d, 2H), 8.40 (s, 1H), 9.45 (s, 1H), 9.50 (d, 1H), 9.60 (t, 1H).

### Example 1006

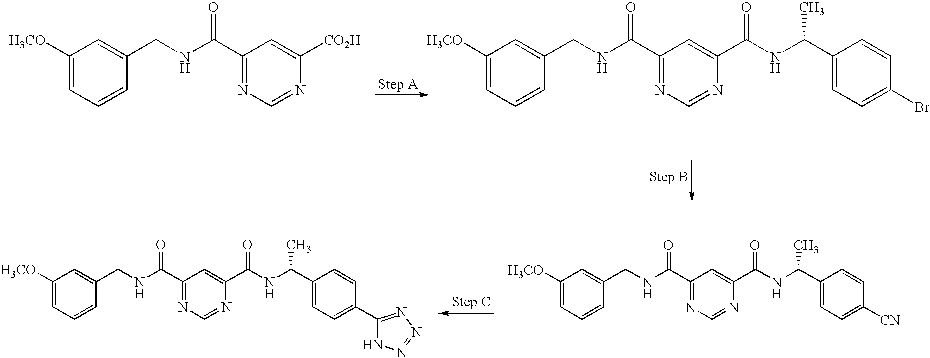

Following the procedure described in Example 1005, except using the enantiomer (R)-1-(4-bromophenyl)ethylamine as amine, the title compound was prepared.

### Example 1007

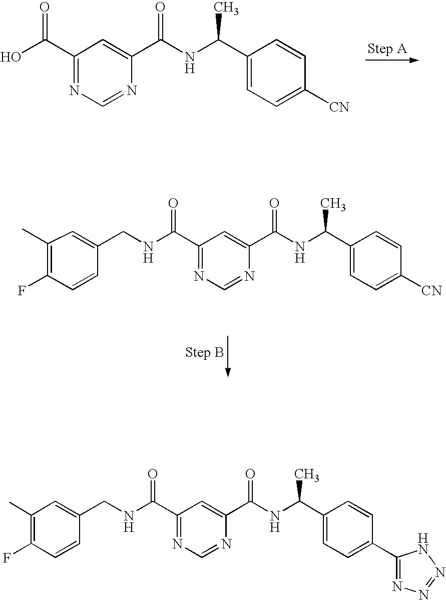

Step A

The title compound from Preparative Example 204 (990 mg) was dissolved in a mixture of anhydrous CH₂Cl₂ (10 mL) and anhydrous THF (10 mL) under argon. To this solution was added isobutylchloroformate (484 μL) After 5 h was added N-methylmorpholine (900 μL) followed by 4-fluoro-3-methylbenzylamine (1 g). The mixture was stirred overnight, concentrated under reduced pressure and diluted with ethyl acetate. The resulting solution was washed with hydrochloric acid (1M), saturated sodium bicarbonate and brine. Flash chromatography (cyclohexane/ethyl acetate 6:4 to 1:1) afforded the intermediate (1.18 g; 84%). [MH]⁺=418.

Step B

The intermediate from step A above (219 mg) and Bu₂SnO (10 mg) were suspended in anhydrous toluene (3 mL) upon which TMSN₃ (150 μL) was added. The mixture was then heated to 110° C. overnight. After cooling to room temperature, the toluene was removed under high vacuum and the remaining residue was chromatographed (dichlormethane/methanol 80:20) to give the title compound (198 mg; 82%) as a colourless solid. [MH]⁺=461.

### Example 1007a-1007φ

Following the procedure described in Example 1007, except using the acid from Preparative Example 204 and the amine indicated in the table below, the title compound was prepared.

| Ex. # | Amine | Product | MS |
|---|---|---|---|
| 1007a | 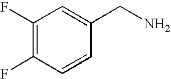 | 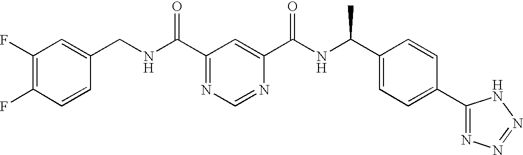 | [MH]⁺ =465 |
| 1007b | 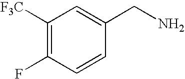 | 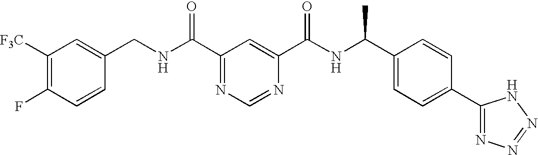 | [MH]⁺ =515 |
| 1007c | 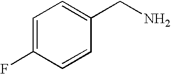 | 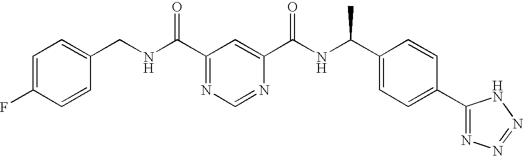 | [MH]⁺ =447 |
| 1007d | 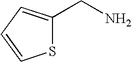 | 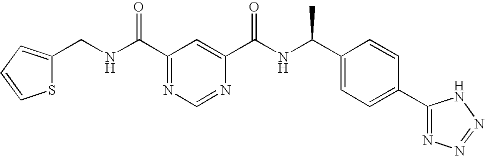 | [MH]⁺ =435 |
| 1007e | 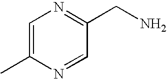 | 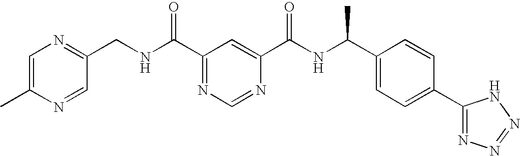 | [MH]⁺ =445 |
| 1007f | 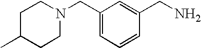 | 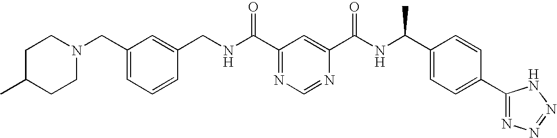 | [MH]⁺ =540 |
| 1007g | 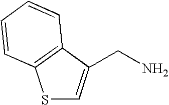 | 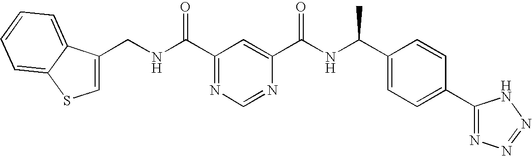 | [MH]⁺ =485 |
| 1007h | 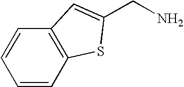 | 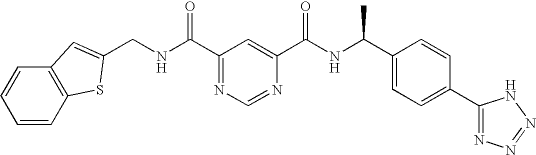 | [MH]⁺ =485 |
| 1007i | 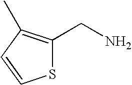 | 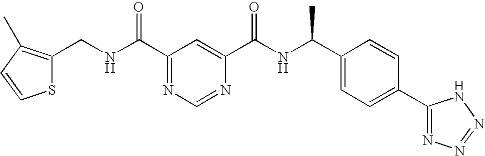 | [MH]⁺ =449 |
| 1007j | 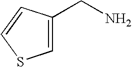 | 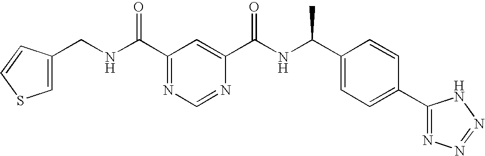 | [MH]⁺ =435 |
| 1007k | 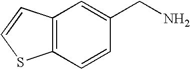 | 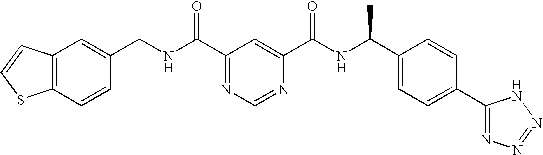 | [MH]⁺ =485 |
| 1007l | 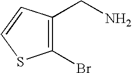 | 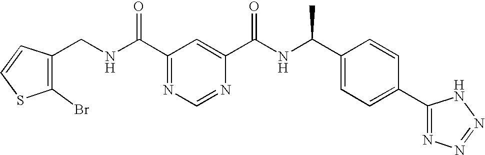 | [MH]⁺ =513/515 |
| 1007m | 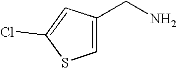 | 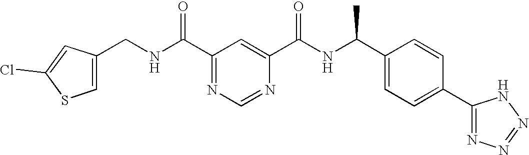 | [MH]⁺ =469 |
| 1007n | 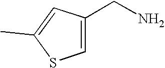 | 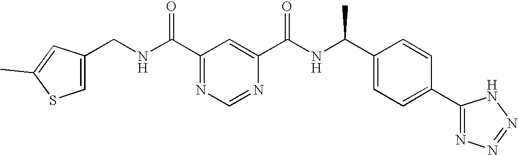 | [MH]⁺ =449 |
| 1007o | 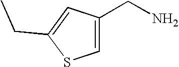 | 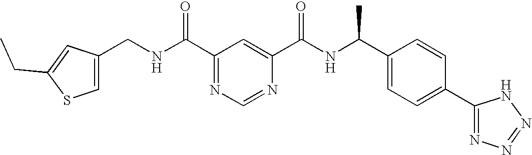 | [MH]⁺ =463 |
| 1007p | 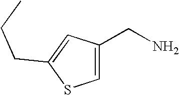 | 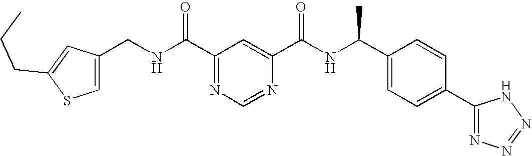 | [MH]⁺ =477 |
| 1007q | 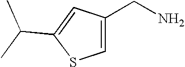 | 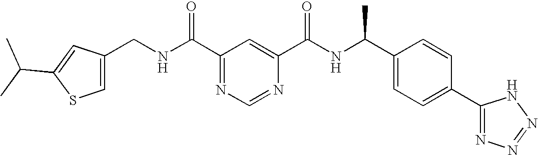 | [MH]⁺ =477 |
| 1007r | 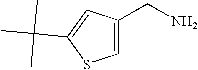 | 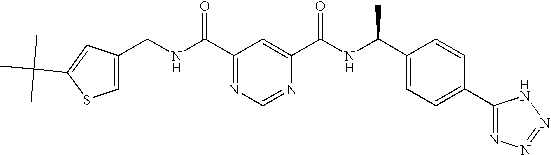 | [MH]⁺ =491 |
| 1007s | 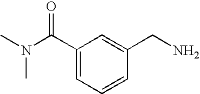 | 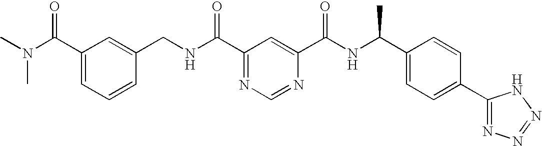 | [MH]⁺ =500 |
| 1007t | 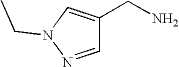 | 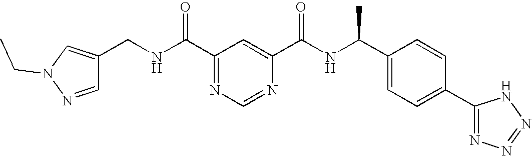 | [MH]⁺ =447 |
| 1007u | 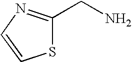 | 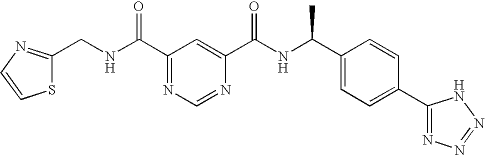 | [MH]⁺ =436 |
| 1007v | 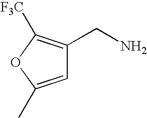 | 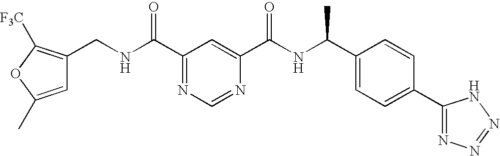 | [MH]⁺ =501 |
| 1007w | 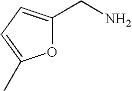 | 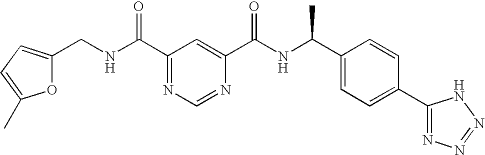 | [MH]⁺ =433 |
| 1007x | 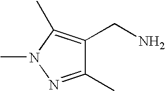 | 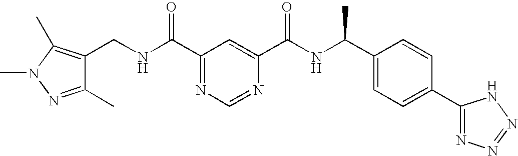 | [MH]⁺ =461 |
| 1007y | 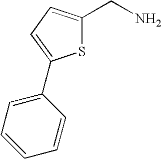 | 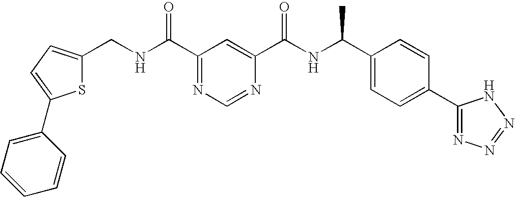 | [MH]⁺ =511 |
| 1007z | 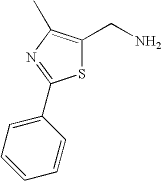 | 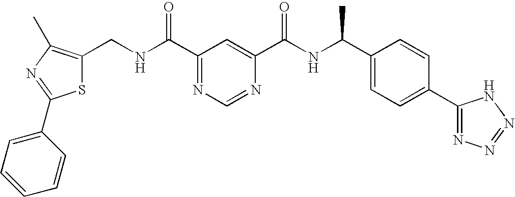 | [MH]⁺ =526 |
| 1007α | 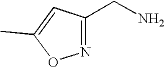 | 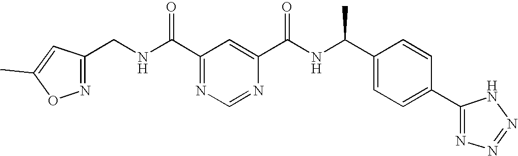 | [MH]⁺ =434 |
| 1007β | 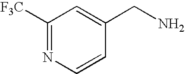 | 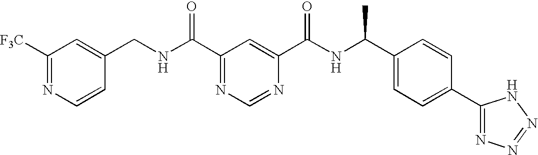 | [MH]⁺ =498 |
| 1007χ | 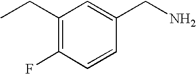 | 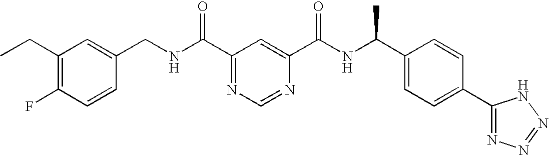 | [MH]⁺ =475 |
| 1007δ | 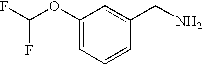 | 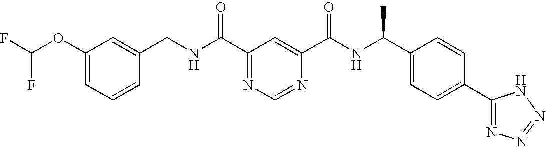 | [MH]⁺ =495 |
| 1007ε | 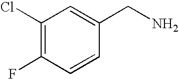 | 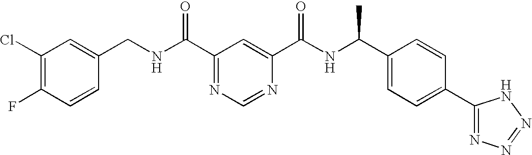 | [MH]⁺ =481 |
| 1007φ | 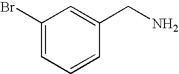 | 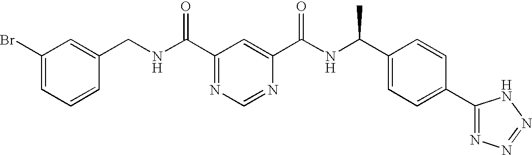 | [MH]⁺ =507/509 |

### Example 1008

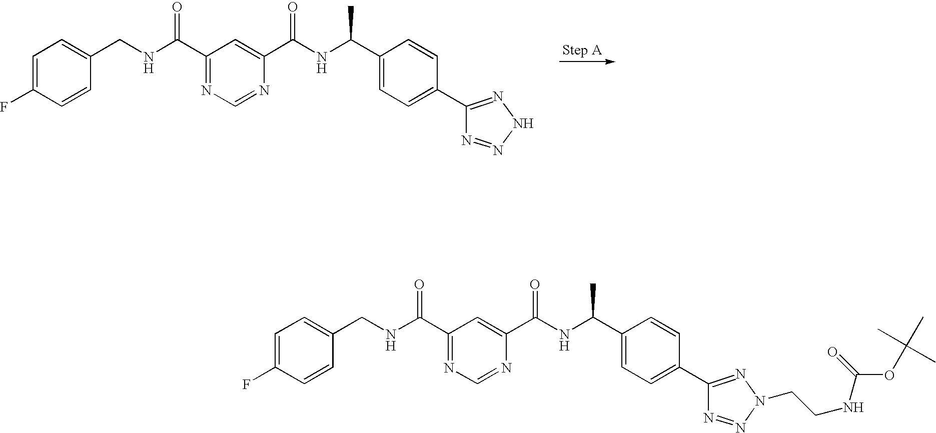 Step A

To a suspension of potassium carbonate (415 mg) in dry N,N-dimethylformamide (9 mL) were successively added the intermediate from Example 1007c (133 mg) and (2-bromo-ethyl)-carbamic acid tert-butyl ester (134 mg). The resulting mixture was stirred at room temperature for 112 h interrupted by further addition of portions of (2-bromo-ethyl)-carbamic acid tert-butyl ester (134 mg) after 27, 47 and 97 h. The solvent was removed under reduce pressure, all inorganic salts were removed by flash filtration (silica, dichloromethane/methanol) and the remaining residue was purified by flash chromatography (silica, cyclohexane/ethyl acetate) to afford the title compound (125 mg, 71%) and the corresponding N1-isomer (15 mg; 8%). [MH]⁺=590.

### Example 1009

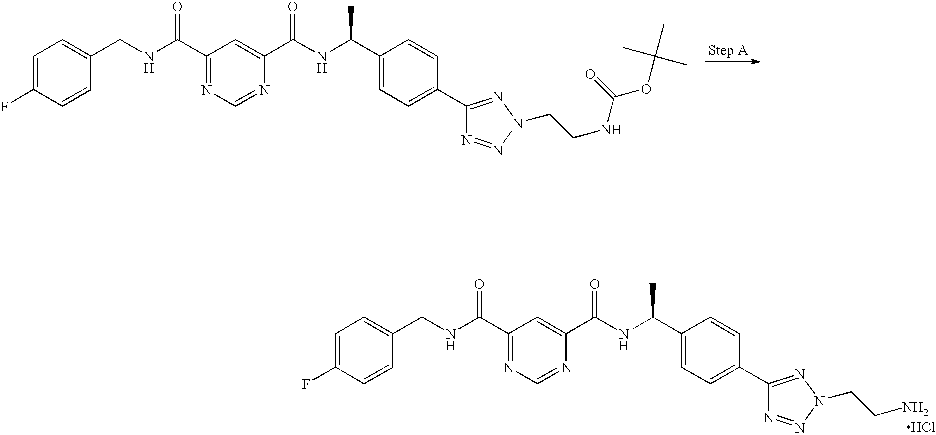 Step A

The title compound from Example 1008 (16.3 mg) was suspended in a 4M solution of hydrochloric acid in dioxane (600 μL). The resulting reaction mixture was stirred at room temperature for 30 min and then concentrated under reduce pressure to afford the title compound as the hydrochloric acid salt (14.5 mg; >99%). [M-Cl]⁺=490.

### Example 1010

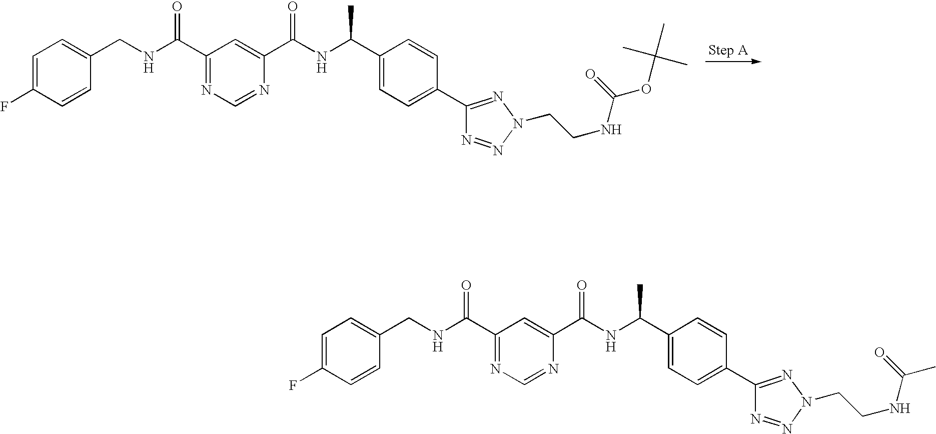 Step A

The title compound from Example 1008 (16.3 mg) was suspended in a 4M solution of hydrochloric acid in dioxane (600 μL). The resulting reaction mixture was stirred at room temperature for 1 h and then concentrated under reduce pressure. The remaining solid residue was dissolved in dry pyridine (500 μL), a solution of 100 mM solution of acetyl chloride in dry dichloromethane (600 μL) was added and the reaction mixture was placed on a shaker for 22 h at ˜900 rpm. The mixture concentrated under reduce pressure and purified by flash chromatography (silica, dichloromethane/methanol) to afford the title compound (11.7 mg; 78%). [MH]⁺=532.

### Example 1011

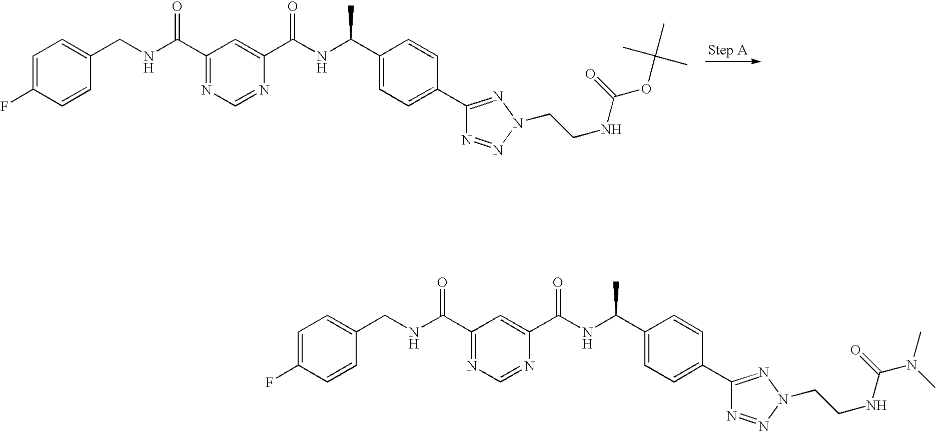 Step A

The title compound from Example 1008 (16.3 mg) was suspended in a 4M solution of hydrochloric acid in dioxane (600 μL). The resulting reaction mixture was stirred at room temperature for 1 h and then concentrated under reduce pressure. The remaining solid residue was dissolved in dry pyridine (500 μL), a solution of 100 mM solution of dimethylcarbamoyl chloride in dry dichloromethane (600 μL) was added and the reaction mixture was placed on a shaker for 22 h at ˜900 rpm. The mixture concentrated under reduce pressure and purified by flash chromatography (silica, dichloromethane/methanol) to afford the title compound (12.2 mg; 78%). [MH]⁺=561.

### Example 1012

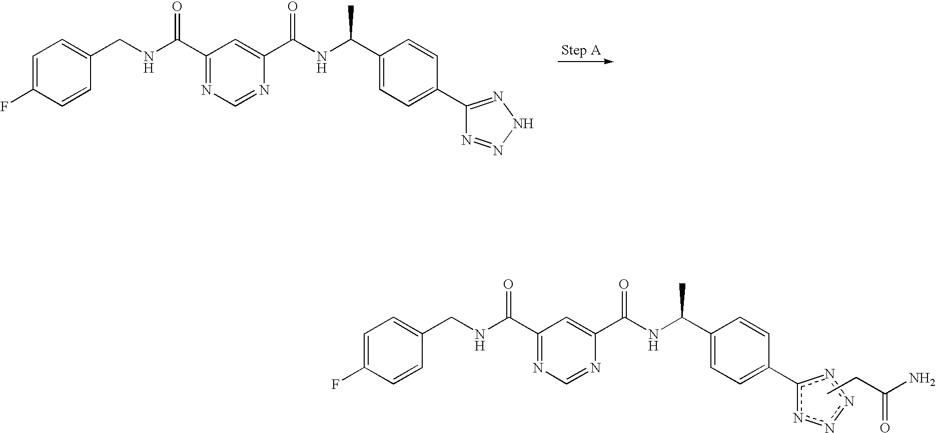 Step A

To a suspension of potassium carbonate (69.1 mg) in dry N,N-dimethylformamide (1.5 mL) were successively added the title compound from Example 1007c (22.3 mg) and 2-bromoacetamide (14.1 mg). The resulting mixture was stirred at room temperature for 19 h, filtered through glass wool and concentrated under reduce pressure. The remaining residue was purified by flash chromatography (silica, dichloromethane/methanol) to afford the title compound (17.4 mg; 69%) as a ˜90:10 mixture of the N2,N1-isomers. [MH]⁺=504.

### Example 1013

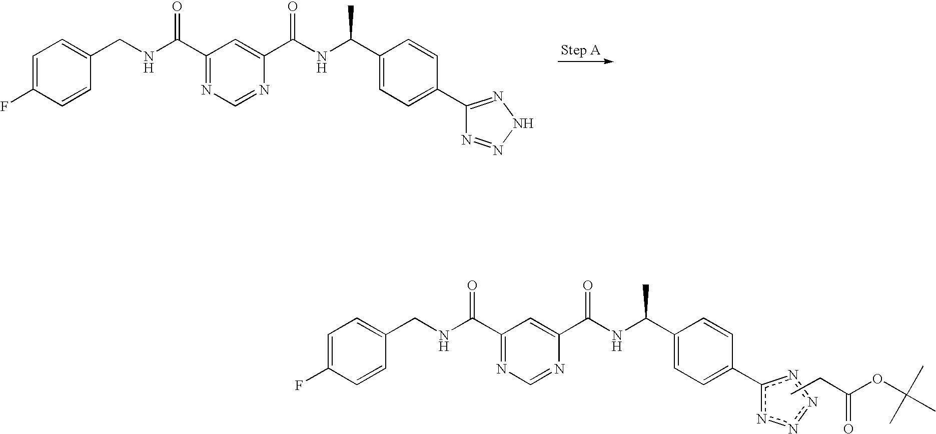 Step A

To a suspension of potassium carbonate (69.1 mg) in dry N,N-dimethylformamide (1.5 mL) were successively added the title compound from Example 1007c (22.3 mg) and bromoacetic acid tert-butyl ester (16.7 μL). The resulting mixture was stirred at room temperature for 16 h and then concentrated under reduce pressure. The remaining residue was purified by flash filtration (silica, dichloromethane/methanol) to afford the title compound (22.3 mg; 79%) as a ˜93:7 mixture of the N2,N1-isomers. [MH]⁺=561.

### Example 1014

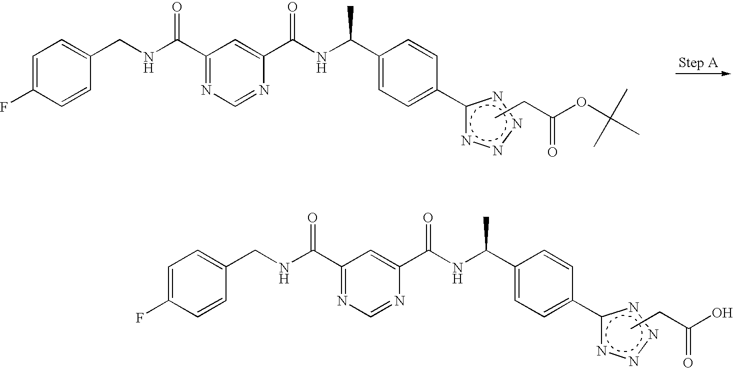 Step A

To a suspension of the title compound from Example 1013 (14.7 mg) in dry dichloromethane (400 μL) was added trifluoroacetic acid (100 μL). The resulting reaction mixture was shaken at room temperature for 5 h and then concentrated under reduce pressure. The remaining residue was purified by flash chromatography (silica, dichloromethane/methanol) to afford the title compound (14.5 mg; 89%, mixture of the N2,N1-isomers) containing ˜1 equivalent trifluoroacetic acid. [MH]⁺=505.

### Example 1015

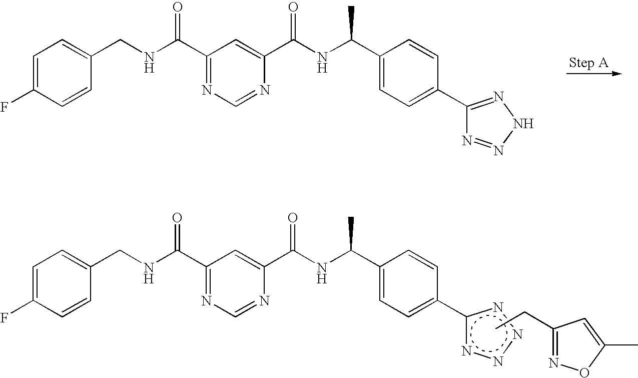 Step A

To a suspension of potassium carbonate (69.1 mg) in dry N,N-dimethylformamide (1.5 mL) were successively added the title compound from Example 1007c (22.3 mg) and 3-bromomethyl-5-methyl-isoxazole (18.1 mg). The resulting mixture was stirred at room temperature for 17 h and then concentrated under reduce pressure. The remaining residue was purified by flash filtration (silica, dichloromethane/methanol) to afford the title compound (21.7 mg; 80%) as a ˜90:10 mixture of the N2,N1-isomers. [MH]⁺=542.

### Example 1016

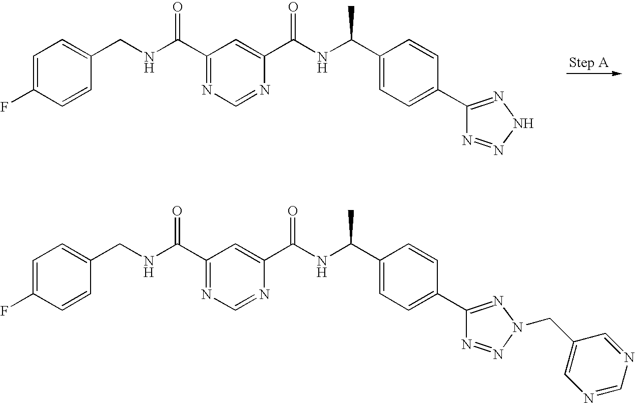 Step A

To a solution of commercially available 5-methyl-pyrimidine in tetrachloromethane (20 mL) were successively added N-bromsuccinimide (392 mg) and dibenzoyl peroxide (24 mg). The resulting suspension was heated to reflux for 23 h in the dark, cooled to −20° C., filtered and filtered, concentrated under reduce pressure at 25° C. and purified by flash chromatography (silica, cyclohexane/ethyl acetate). The obtained material was dissolved in dry N,N-dimethylformamide (1.5 mL) and added to a suspension of the title compound from Example 1007c (22.3 mg) and potassium carbonate (69.1 mg) in dry N,N-dimethylformamide (1.5 mL). The resulting mixture was stirred at room temperature for 20 h and then concentrated under reduce pressure. The remaining residue was purified by flash chromatography (silica, dichloromethane/methanol) to afford the title compound (19 mg; 67%) as a single isomer, containing ˜20 mol % succinimide. [MH]⁺=539.

### Example 1017

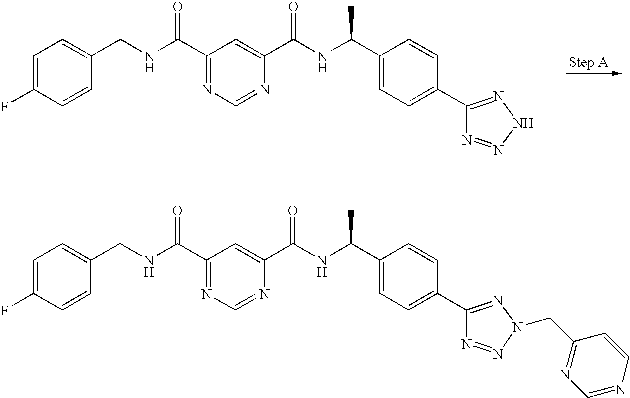 Step A

To a solution of commercially available 4-methyl-pyrimidine in tetrachloromethane (20 mL) were successively added N-bromsuccinimide (392 mg) and dibenzoyl peroxide (24 mg). The resulting suspension was heated to reflux for 23 h in the dark, cooled to −20° C., filtered and filtered, concentrated under reduce pressure at 25° C. and purified by flash chromatography (silica, cyclohexane/ethyl acetate). The obtained material was dissolved in dry N,N-dimethylformamide (1.5 mL) and added to a suspension of the title compound from Example 1007c (22.3 mg) and potassium carbonate (69.1 mg) in dry N,N-dimethylformamide (1.5 mL). The resulting mixture was stirred at room temperature for 22 h and then concentrated under reduce pressure. The remaining residue was purified by flash chromatography (silica, dichloromethane/methanol) to afford the title compound (15 mg; 56%) as a single isomer. [MH]⁺=539.

### Example 1018

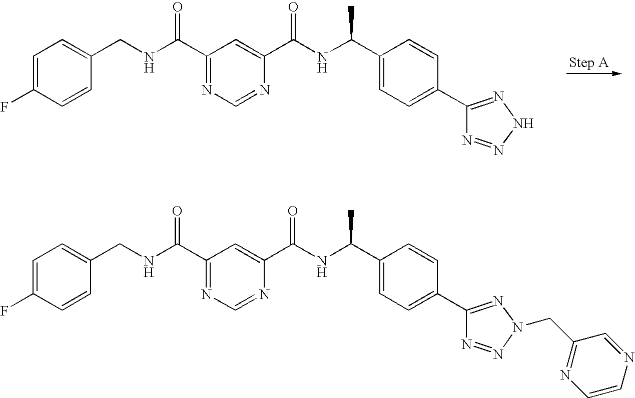 Step A

To a suspension of triphenylphosphine polystyrene (3 gm, 1 mmol/gm) in dry dichloromethane (20 ml) was slowly added bromine (154 μL). The resulting mixture was stirred at room temperature for 10 min, a solution of commercially available pyrazin-2-yl-methanol (114 mg) in dry dichloromethane (10 ml) was added and stirring at room temperature was continued for 21½ h. The mixture was filtered, concentrated under reduce pressure at 20° C. and purified by flash chromatography (silica, cyclohexane/ethyl acetate). The obtained material was dissolved in dry N,N-dimethylformamide (1.5 mL) and added to a suspension of the title compound from Example 1007c (22.3 mg) and potassium carbonate (69.1 mg) in dry N,N-dimethylformamide (1.5 mL). The resulting mixture was stirred at room temperature for 16 h and then concentrated under reduce pressure. The remaining residue was purified by flash chromatography (silica, dichloromethane/methanol) to afford the title compound (6.8 mg; 25%) as a single isomer. [MH]⁺=539.

### Example 1019

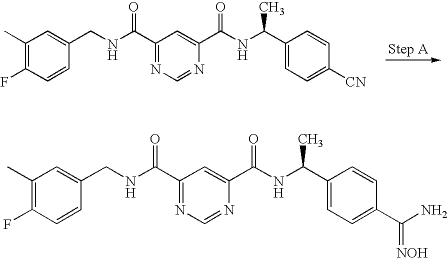 Step A

A solution of the intermediate from Example 1007, Step A (742 mg), NH₂OH.HCl (2 g) and NaHCO₃ (2 g) in ethanol (60 mL) and water (10 mL) was refluxed overnight. The mixture was concentrated under reduced pressure and diluted with ethyl acetate and the resulting solution was washed with brine. Flash chromatography (cyclohexane/ethyl acetate 2:8 to 0:1) afforded the title compound (711 mg; 89%) as a clourless foam. [MH]⁺=451.

### Example 1020

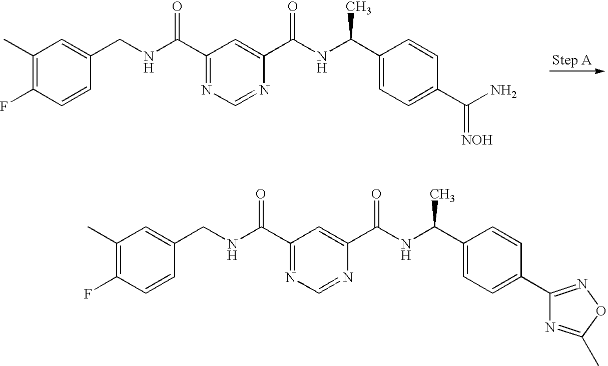 Step A

A solution of the title compound from Example 1019 (62 mg) in acetic acid anhydride (2 mL) was heated to 100° C. overnight. The mixture was concentrated under reduced pressure and diluted with ethyl acetate and the resulting solution was washed with saturated NaHCO₃ solution and brine. Flash chromatography (cyclohexane/ethyl acetate 1:1) afforded the title compound (41 mg; 62%) as a clourless solid. [MH]⁺=475.

### Example 1021

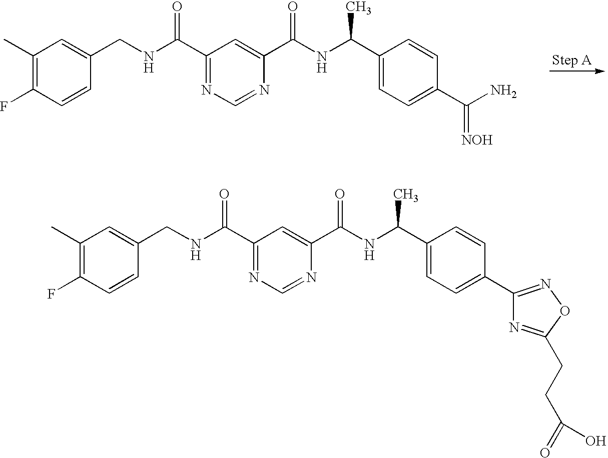 Step A

A solution of the title compound from Example 1019 (80.6 mg) and succinic anhydride (27 mg) was heated in xylene (4 mL) to reflux overnight. The mixture was absorbed on silica and purified by flash chromatography (cyclohexane/ethyl acetate 2:8 to 0:1) to afford the title compound (36.3 mg; 38%) as a clourless solid. [MH]⁺=533.

### Example 1022

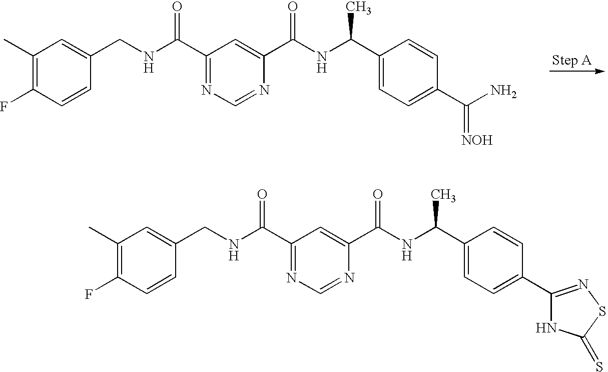 Step A

A solution of the title compound from Example 1019 (76.6 mg) and KOH (41 mg) was heated in ethanol (0.5 mL) and carbon disulfide (3 mL) to reflux overnight. The mixture was concentrated under reduced pressure and diluted with ethyl acetate and the resulting solution was washed with 10% aqueous citric acid solution and brine. Flash chromatography (cyclohexane/ethyl acetate 4:6 to 3:7) afforded the title compound (84 mg; 97%) as a bright yellow solid. [MH]⁺=509.

### Example 1023

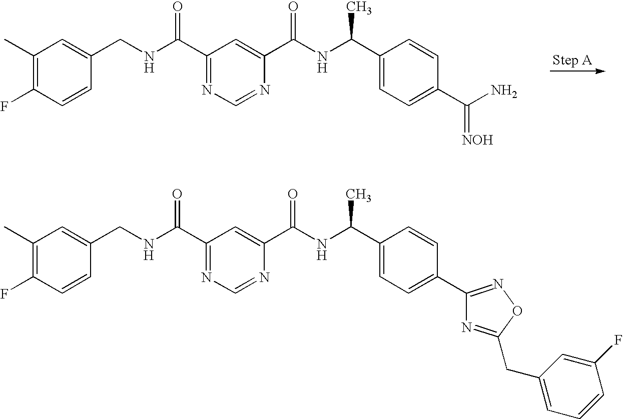 Step A

3-Fluorophenylacetic acid (111 mg) and carbonyldiimidazole (120 mg) were heated at 80° C. for 1½ h. The mixture was cooled to room temperature and the title compound from Example 1019 (60 mg) and KHCO₃ (200 mg) were added. The mixture was refluxed overnight, concentrated under reduced pressure and diluted with ethyl acetate and the resulting solution was washed with 10% aqueous citric acid solution and brine. Flash chromatography (cyclohexane/ethyl acetate 6:4) afforded the title compound (80.8 mg; quantitative) as slowly crystallizing colourless oil. [MH]⁺=569.

### Example 1024

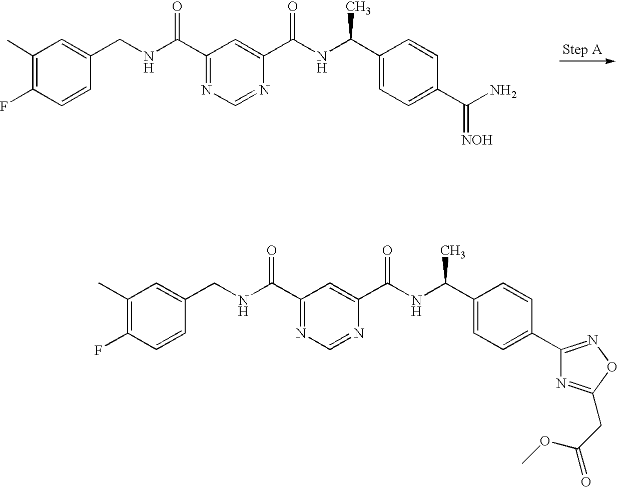 Step A

A solution of the title compound from Example 1019 (95 mg) and methyl-3-chloro-3-oxopropionate (290 μL) was heated in dry pyridine (3 mL) at 50° C. for 3 d. The mixture was concentrated under reduced pressure and diluted with ethyl acetate and the resulting solution was washed with 10% aqueous citric acid solution and brine. Flash chromatography (cyclohexane/ethyl acetate 1:1 to 4:6) afforded the title compound (61.5 mg; 55%) as yellow amorphous mass. [MH]⁺=533.

### Example 1025

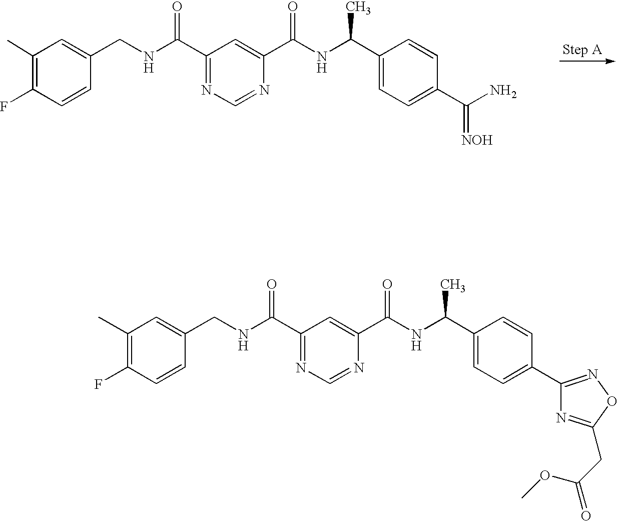 Step A

A solution of the title compound from Example 1024 (30 mg) was heated in a pressure tube in ammonia (6N in methanol) 60° C. overnight. The mixture was concentrated and preparative thin layer chromatography (dichloromethane/methanol 9:1) afforded the title compound (15.1 mg; 52%) as a colourless solid. [MH]⁺=518.

### Example 1026

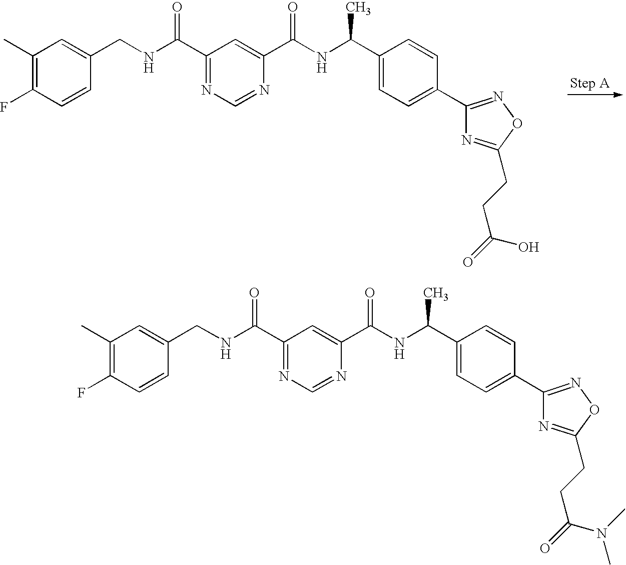 Step A

A solution of the title compound from Example 1021 (52 mg) and PyBroP (100 mg) in DMF (2 mL) was added dimethylamine (2M in THF; 0.5 mL). The mixture was stirred overnight and diluted with ethyl acetate and the resulting solution was washed with 10% aqueous citric acid solution and brine. Flash chromatography (dichloromethane/methanol 95:5) yielded the title compound (46.1 mg; 84%) as colourless crystals. [MH]⁺=560.

### Example 1027

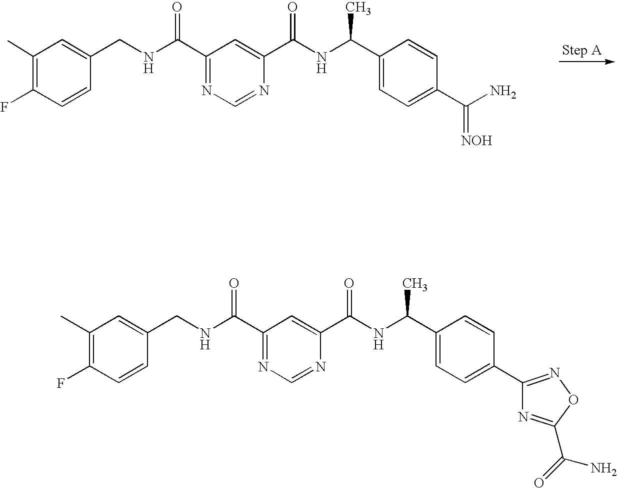 Step A

A solution of the title compound from Example 1019 (82 mg), catalytical amounts of dimethylaminopyridine and methyl chlorooxoacetate (25 μL) was stirred in dry pyridine (2 mL) overnight. The mixture was absorbed on silica and purified by flash chromatography (dichloromethane/acetone 95:5) to afford the ester, which was diluted in ammonia (0.5M in dioxane; 10 mL) and heated in a sealed tube to 60° C. overnight. Preparative thin layer chromatography (dichloromethane/methanol 95:5) afforded the title compound (37.8 mg; 41%) as a clourless solid. [MH]⁺=504.

### Example 1028

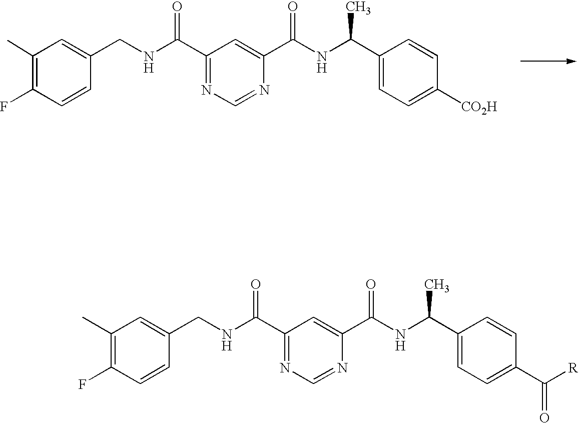

The title compound from Example 1000 and the amine according the table below were coupled with PyBop at room temperature in dry THF. Purification by silica gel chromatography to afforded the title compound indicated in the table below.

| Ex. # | Amine | Product | MS |
|---|---|---|---|
| 1028a | 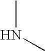 | 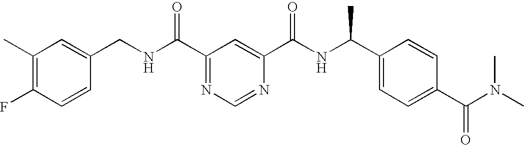 | [MH]⁺ = 464 |
| 1028b | 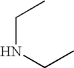 | 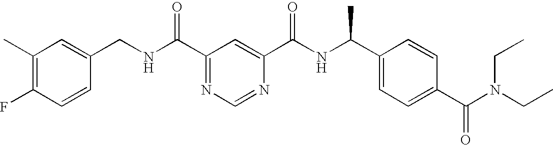 | [MH]⁺ = 492 |
| 1028c | 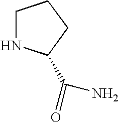 | 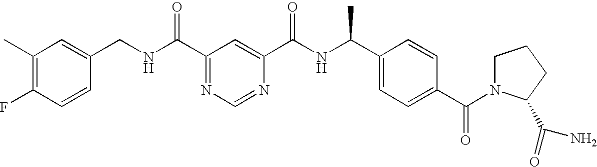 | [MH]⁺ = 533 |
| 1028d | 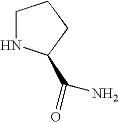 | 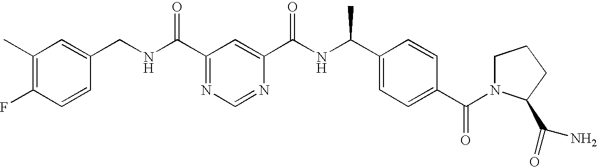 | [MH]⁺ = 533 |
| 1028e | 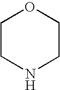 | 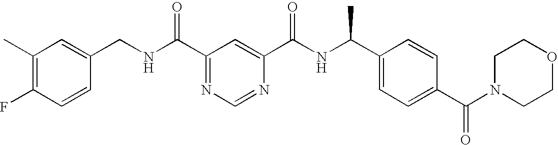 | [MH]⁺ = 506 |

### Example 1029

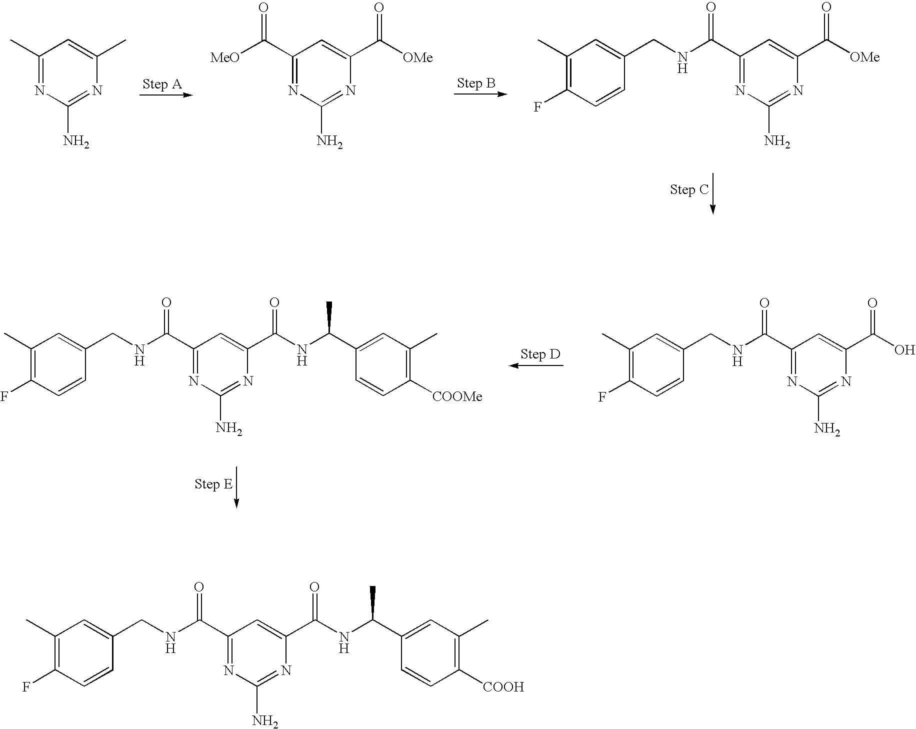 Step A

To commercially available 4,6-dimethyl-pyrimidin-2-ylamine (6.0 g) in water (400 mL) was added a solution of sodium hydroxide (1.3 g in 5 mL water) and heated at 80° C. for 10 min. Then potassium permanganate (15 g) was added and heated between 85° C. to 90° C. for 1 h. Potassium permanganate (15 g) was again added and mixture was heated for another 2 h. The mixture was cooled to room temperature and filtered through Celite® and then acidified to pH ˜2. The mixture was concentrated to 20% of the original volume and the solid was filtered and dried. To solid was dissolved in methanol (200 mL) and saturated with dry hydrogen chloride gas and the mixture was heated to reflux for 24 h. The mixture was concentrated to an oil and then taken up in dichloromethane and the organic phase was washed with saturated NaHCO₃ and then dried over MgSO₄, filtered and concentrated to give a solid which was purified by column chromatography (silica, 10% methanol/dichloromethane) to give the intermediate (0.41 g). [MH]⁺=212.

Step B

A solution of the intermediate from step A above (0.24 g) in N,N-dimethylformamide (3 mL) was added 4-fluoro-3-methyl-benzylamine (0.15 g) dissolved in N,N-dimethylformamide (1 mL) and the mixture was stirred at 80° C. for 15 h, concentrated and then purified by column chromatography (silica, 10% methanol/dichloromethane) to afford the intermediate (0.15 g; 28%) as a colourless foam. [MH]⁺=319.

Step C

A solution of the intermediate from step B above (0.15 g) in tetrahydrofuran (2 mL) was added a 1N potassium hydroxide solution (2 mL) and was stirred for 24 h. The mixture was concentrated and purified by column chromatography (silica, 10% methanol/dichloromethane) to afford the intermediate (60 mg; 42%). [MH]⁺=305.

Step D

If one were to add to a solution of the intermediate of Step C above (20 mg) in N,N-dimethylformamide (0.5 mL), N-methylmorpholine (15 μL) and cool the mixture (−40° C.) under nitrogen, and then add isobutyl chloroformate (10 μL) and then stir the mixture at between −40° C. to −20° C. for 1.5 h then add the title compound from preparative example 100 (13 mg) dissolved in tetrahydrofuran (0.5 mL) and then stir the mixture at −40° C. to −20° C. for 1 h and and then slowly warm to room temperature and then add water (1-2 drops) and then concentrate and then purify by preparative thin layer chromatography (silica, 10% methanol/CH₂Cl₂) one would obtain the resulting methyl ester.

Step E

If one were to dissolve the intermediate from Step D above in tetrahydrofuran and then add a slight excess of 1N potassium hydroxide solution and then water and then stir the mixture at room temperature for 15 h and then concentrate and then add 1N hydrochloric acid and then concentrate and then purify the resulting solid by preparative thin layer chromatography (silica, 10% methanol/dichloromethane) one would get the title compound.

### Example 1030

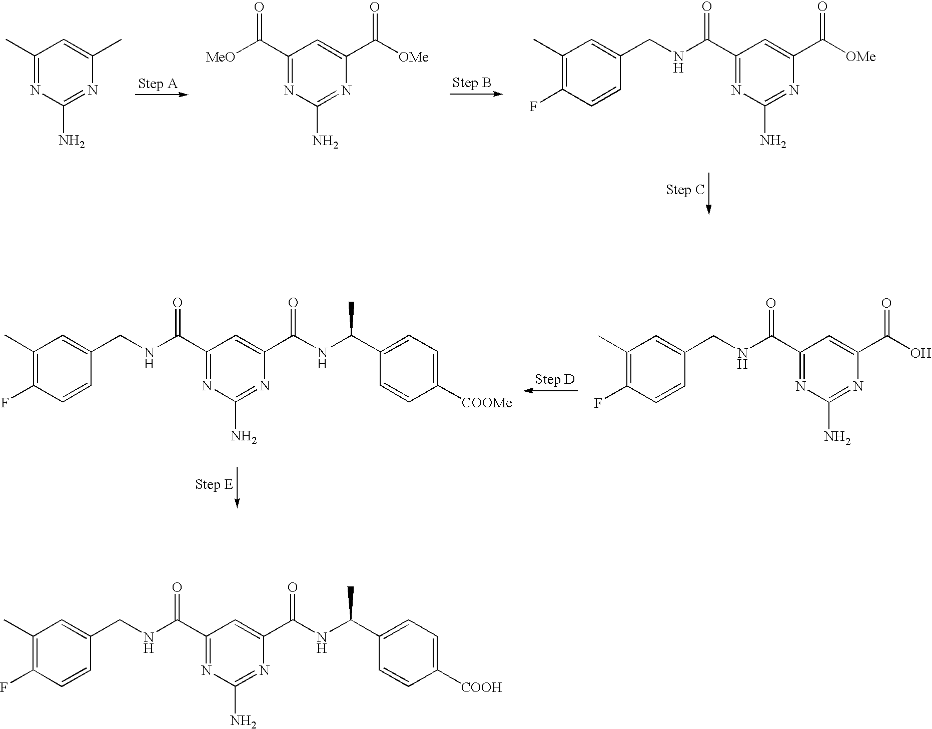 Step A

To commercially available 4,6-dimethyl-pyrimidin-2-ylamine (6.0 g) in water (400 mL) was added a solution of sodium hydroxide (1.3 g in 5 mL water) and heated at 80° C. for 10 min. Then potassium permanganate (15 g) was added and heated between 85° C. to 90° C. for 1 h. Potassium permanganate (15 g) was again added and mixture was heated for another 2 h. The mixture was cooled to room temperature and filtered through Celite® and then acidified to pH ˜2. The mixture was concentrated to 20% of the original volume and the solid was filtered and dried. To solid was dissolved in methanol (200 mL) and saturated with dry hydrogen chloride gas and the mixture was heated to reflux for 24 h. The mixture was concentrated to an oil and then taken up in dichloromethane and the organic phase was washed with saturated NaHCO₃ and then dried over MgSO₄, filtered and concentrated to give a solid which was purified by column chromatography (silica, 10% methanol/dichloromethane) to give the intermediate (0.41 g). [MH]⁺=212.

Step B

A solution of the intermediate from step A above (0.24 g) in N,N-dimethylformamide (3 mL) was added 4-fluoro-3-methyl-benzylamine (0.15 g) dissolved in NAN-dimethylformamide (1 mL) and the mixture was stirred at 80° C. for 15 h, concentrated and then purified by column chromatography (silica, 10% methanol/dichloromethane) to afford the intermediate (0.15 g; 28%) as a colourless foam. [MH]⁺=319.

Step C

A solution of the intermediate of Step B above (0.15 g) in tetrahydrofuran (2 mL) was added a 1N potassium hydroxide solution (2 mL) and was stirred for 24 h. The mixture was concentrated and purified by column chromatography (silica, 10% methanol/dichloromethane) to afford the intermediate (60 mg; 42%). [MH]⁺=305.

Step D

If one were to add to a solution of the intermediate of Step C above (20 mg) in N,N-dimethylformamide (0.5 mL) N-methylmorpholine (15 μL) and cool the mixture (−40° C.) and then add isobutyl chloroformate (10 μL) and then stir at between −40° C. to −20° C. for 1.5 h and then add the title compound from Preparative Example 100 (13 mg) dissolved in tetrahydrofuran (0.5 mL) and then stir at −40° C. to −20° C. for 1 h and then add water (1-2 drops) and stir for 1 h and then concentrate and purify by preparative thin layer chromatography (silica, 10% methanol/dichloromethane) one would get the resulting methyl ester.

Step E

If one were to dissolve the intermediate from Step D above in tetrahydrofuran and add a slight excess of 1N potassium hydroxide solution and water and then stir the mixture at room temperature for 15 h and then concentrate the mixture and add to the resulting solid 1N hydrochloric acid then concentrate and then purify by preparative thin layer chromatography (silica, 10% methanol/dichloromethane) one would get the title compound.

### Example 1031

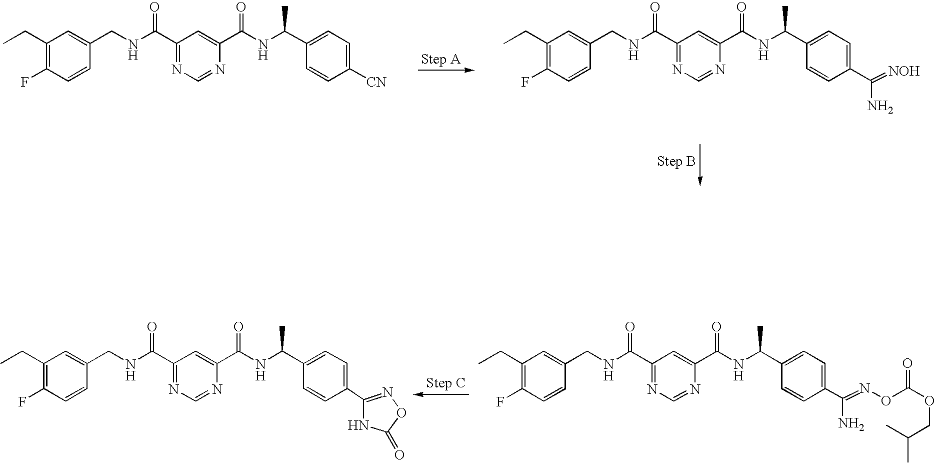 Step A

The intermediate from Preparative Example 1007_{χ} (41.8 mg) was refluxed with hydroxylamine (60 mg hydrochloride salt, neutralized with grounded potassium hydroxide in ethanol) in ethanol (3 mL) overnight. The reaction mixture was concentrated to dryness to give the intermediate as a colourless solid, which is utilized in next step without further purification.

Step B

The intermediate from step A above was dissolved in dimethylformamide (1 mL), and cooled to 0° C. in an ice bath. Pyridine (9 μL) was added followed by the addition of isobutyl chloroformate (13.7 μL). The reaction was kept at same temperature for 30 min, and concentrated to dryness to give the intermediate as a brown oil.

Step C

To the intermediate from step B above was added chlorobenzene (3 mL) and refluxed for 3 h. The reaction mixture was concentrated to dryness. The crude material was purified by column chromatography to furnish the title compound (28 mg; 60% over 3 steps) as an off-white solid. [MH]⁺=491.

### Example 1032

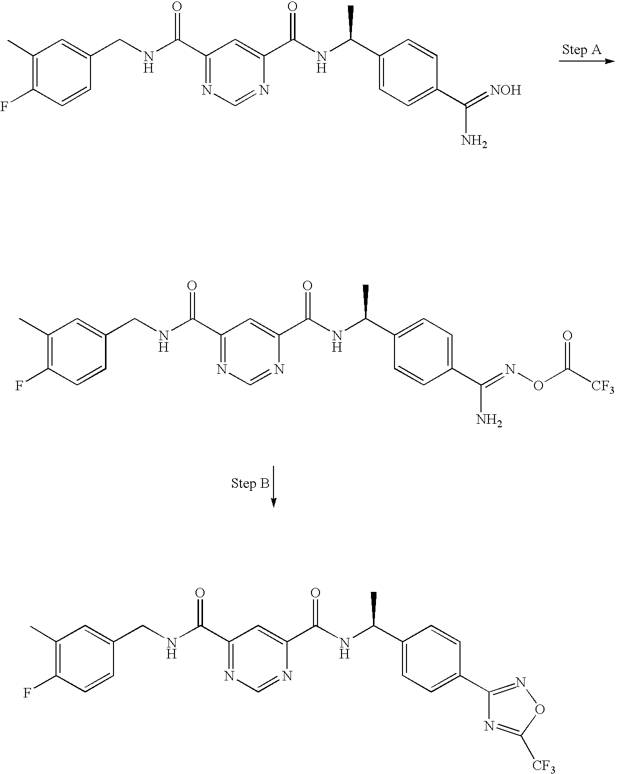 Step A

The title compound from Example 1019 (67.5 mg) was dissolved in tetrahydrofuran (2 mL), and cooled to 0° C. in an ice bath. Pyridine (15 μL) was added followed by the addition of trifluoroacetic anhydride (24 μL). The reaction was kept for 2 h, and concentrated to dryness to give the intermediate, which was used without further purification.

Step B

The intermediate from step A above was added chlorobenzene and refluxed overnight. The reaction mixture was concentrated to dryness. The crude material was purified by column chromatography to furnish the title compound (50 mg). [MH]⁺=529.

### Example 1033

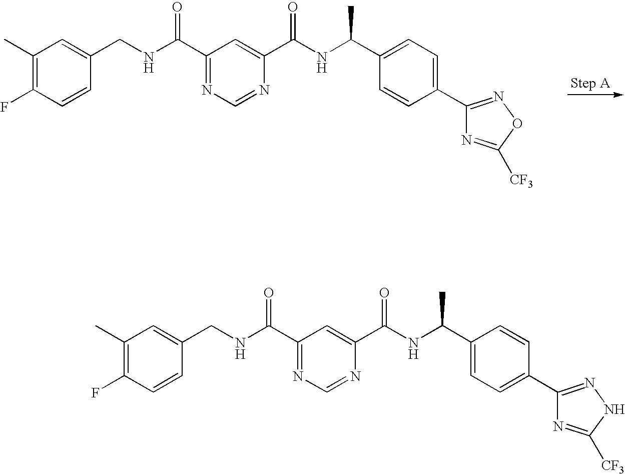 Step A

The title compound from Example 1032 (38 mg) in methanol was added hydrazine (0.1 mL). The reaction mixture was stirred at rt for 2 days and then concentrated to dryness. The crude material was purified by column chromatography to furnish the title compound (10 mg). [MH]⁺=528.

### Example 1034

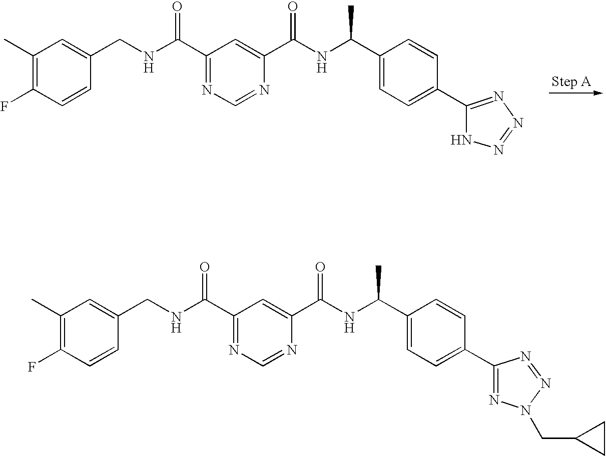 Step A

The title compound from Example 1007 (99 mg), cyclopropylmethyl bromide (25 μL) and K₂CO₃ (45 mg) were combined in DMF (1.5 mL) and stirred at room temperature for 12 h. The mixture was then concentrated under high vacuum and the remaining residue was chromatographed (dichlormethane/methanol 98:2) to give the title compound (50 mg; 55%) as a colourless solid. [MH]⁺=416.

### Example 1035

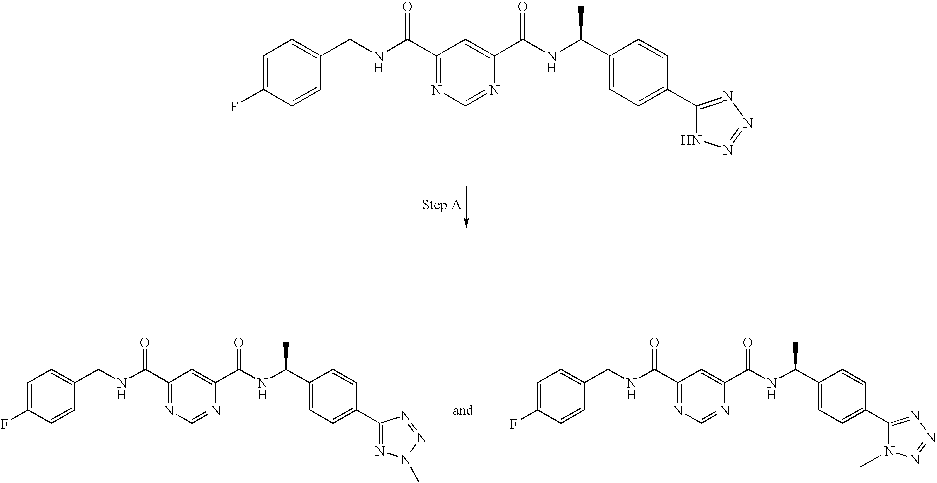 Step A

The title compound from Example 1007c (120 mg), methyl iodide (20 μL) and K₂CO₃ (55 mg) were combined in DMF (5 mL) and stirred at room temperature for 12 h. The mixture was then concentrated under high vacuum and the remaining residue was chromatographed (dichlormethane/methanol 98:2) to give the 2-methyl isomer (22 mg; 18%) and 1-methyl isomer (8 mg; 6%) as colourless solids, respectively. [MH]⁺=461.

### Example 1036

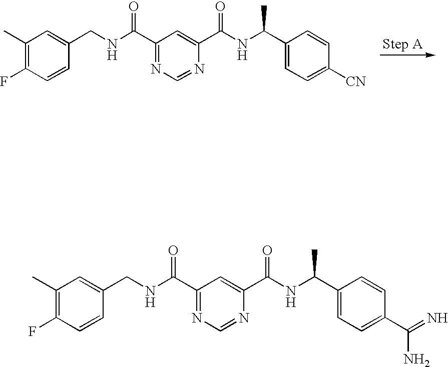 Step A

The title compound from Example 1007 (150 mg) was dissolved in anhydrous methanol (10 mL) and cooled to 0° C. To this stirring solution was bubbled anhydrous HCl gas for 3 minutes upon which the reaction vessel was sealed and placed in the freezer for 12 h. The reaction was then warmed to room temperature and concentrated under reduced pressure upon which the resulting residue was dissolved in ammonia (7M in methanol; 10 mL) and stirred at room temperature for additional 12 h. The mixture was concentrated under reduced pressure at the residue was chromatographed (dichlormethane/methanol 80:20) to give the title compound (60 mg; 39%) as a colourless solid. [MH]⁺=435.

### Example 1037

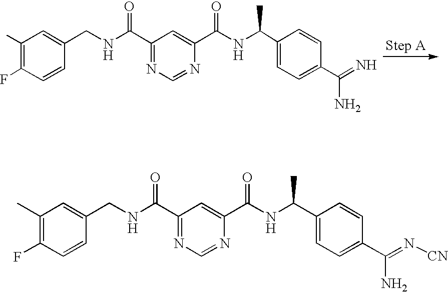 Step A

The title compound from Example 1036 (36 mg) was dissolved in anhydrous dichloromethane (1 mL) and combined with CNBr (11 mg) and diisopropylethyl amine (16 μL) with stirring. LC-MS showed that the reaction had only proceeded by ˜10% after 6 h so an additional amount of CNBr (50 mg) was added. The mixture was stirred for 12 h, concentrated under reduced pressure and chromatographed (dichloromethane) to give the title compound (23 mg; 60%) as a colourless solid. [MH]⁺=460.

### Example 1038

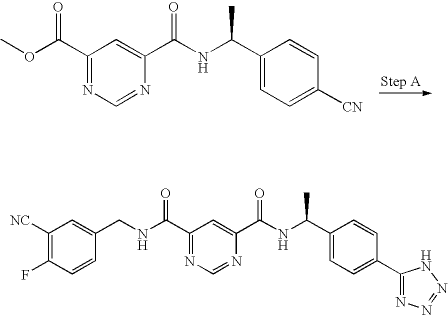

Step A

The title compound from Preparative Example 205 (200 mg) was dissolved in DMF (2 mL) at room temperature. 5-Aminomethyl-2-fluoro-benzonitrile (254 mg) was added and the reaction was stirred at 80° C. for 24 h. No starting material was observed by TLC, (10% MeOH/CH₂Cl₂) the reaction was cooled and the solvent removed in vaccuo to yield a brown solid. This was purified by silica chromatography in (dichloromethane/MeOH 4:1) to yield the title compound (40 mg; 15%) as a colourless solid. [MH]⁺=472.

### Example 1039

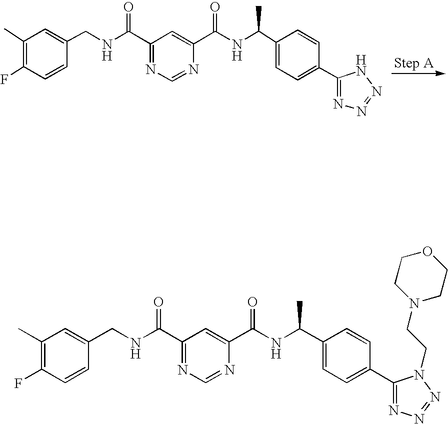

Step A

The title compound from Example 1007 (50 mg) was dissolved in dry THF (3 mL) and triphenyphosphine (43 mg) was added. The reaction was then flushed with nitrogen and 2-morpholin-4-yl-ethanol (21 mg) was added via a syringe. The reaction was then cooled to 0° C. and diethylazodicarboxylate (28 mg) was added dropwise. The reaction was stirred for 24 h, allowing it to warm to room temperature. TLC analysis showed the reaction contained no more starting material. The solvent was evaporated from the reaction and the residue purified by column chromatography to yield the title compound (30 mg) as a colourless solid. [MH]⁺=574.

### Example 1039a-1039b

Following the procedure described in Example 1039, except using the alcohols indicated in the table below, the title compound was prepared.

| | | | |
|---|---|---|---|
| Ex. # | Alcohol | Product | MS |

| | | | |
|---|---|---|---|
| 1039a | 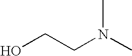 | 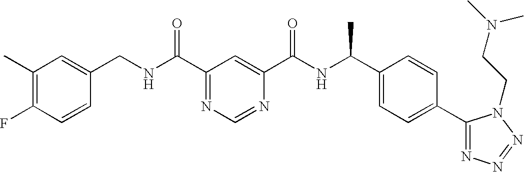 | [MH]⁺ =532 |
| 1039b | 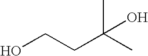 | 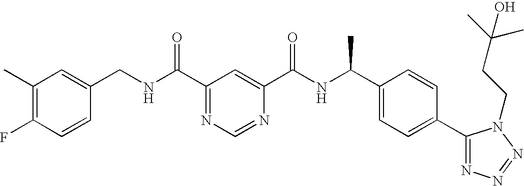 | [MH]⁺ =547 |

### Example 1040a

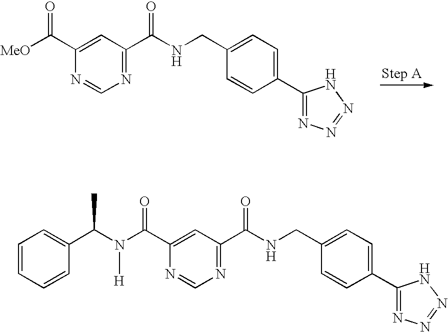 Step A

To a glass vial containing a stir bar was added 65 mg (0.19 mmole) of 6-[4-(1H-Tetrazol-5-yl)-benzylcarbamoyl]-pyrimidine-4-carboxylic acid methyl ester and (R) 1-Phenyl-ethylamine and 1 ml of dimethylformamide and mixture heated at 80° C. under closed; atmosphere for 12 h. The volatile components of the reaction mixture was then removed under reduced pressure the resulting residue was triturated with ether to give the crude amide. The crude product was purified by preparative thin layer chromatography to give the target diamide [MH]⁺=429.

### Preparative Examples 1040b-e

Following the procedure described in Example 1040a, except using the amines listed in the table below, the title compounds was prepared.

| Ex. # | Amine | Ester |
|---|---|---|
| 1040b | 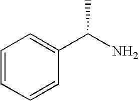 | 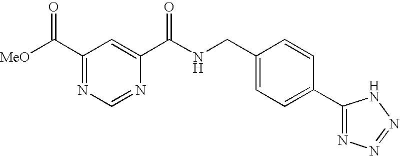 |
| 1040c | 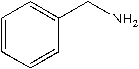 | 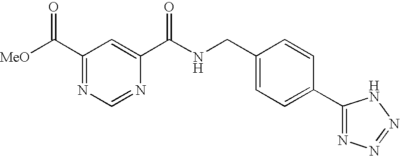 |
| 1040d | 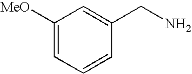 | 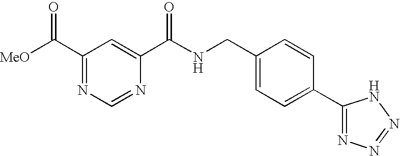 |
| 1040e | 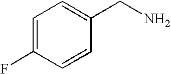 | 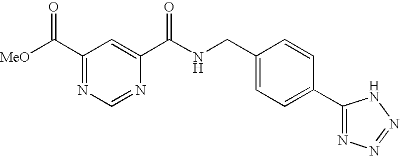 |

| Ex. # | Product | MS |
|---|---|---|
| 1040b | 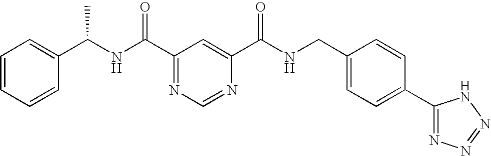 | [MH]⁺ =429 |
| 1040c | 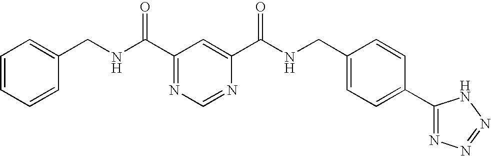 | [MH]⁺ =415 |
| 1040d | 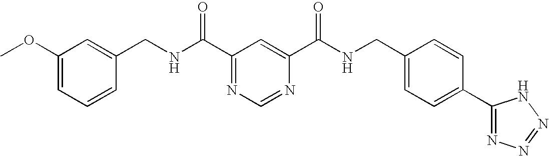 | [MH]⁺ =445 |
| 1040e | 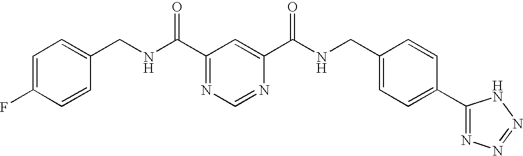 | [MH]⁺ =433 |

What is claimed is:

1. A compound according to Formula (I): 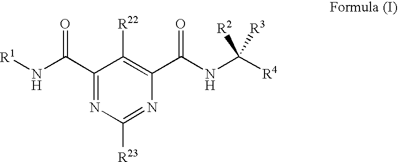
wherein:
R¹ is selected from the group consisting of hydrogen, alkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,
wherein R¹ is optionally substituted one or more times, or
wherein R¹ is optionally substituted by one R¹⁶ group and optionally substituted by one or more R⁹ groups;
R² is selected from the group consisting of hydrogen, alkyl, haloalkyl, fluoroalkyl, cycloalkyl, alkenyl, alkynyl, aryl, heteroaryl, cycloalkyl-alkyl, arylalkyl, heteroarylalkyl, COOR¹⁰, CONR¹⁰R¹¹, SO₂R¹⁰ and SO₂NR¹⁰R¹¹ wherein alkyl, haloalkyl, fluoroalkyl, cycloalkyl, alkenyl, alkynyl, aryl, heteroaryl, cycloalkyl-alkyl, arylalkyl, and heteroarylalkyl are optionally substituted one or more times;
R³ is selected from the group consisting of hydrogen, alkyl, haloalkyl, fluoroalkyl, cycloalkyl, alkenyl, alkynyl, aryl, heteroaryl, cycloalkyl-alkyl, arylalkyl, heteroarylalkyl, COOR¹⁰, CONR¹⁰R¹¹, SO₂R¹⁰ and SO₂NR¹⁰R¹¹ wherein alkyl, haloalkyl, fluoroalkyl, cycloalkyl, alkenyl, alkynyl, aryl, heteroaryl, cycloalkyl-alkyl, arylalkyl, and heteroarylalkyl are optionally substituted one or more times;
R⁴ is selected from the group consisting of alkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl, wherein R⁴ is optionally substituted one or more times;
R⁵ in each occurrence is independently selected from the group consisting of hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;
R⁹ in each occurrence is independently selected from the group consisting of R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,
wherein each R⁹ group is optionally substituted, or
wherein each R⁹ group is optionally substituted by one or more R¹⁴ groups;
R¹⁰ and R¹¹ are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted, or R¹⁰ and R¹¹ when taken together with the nitrogen to which they are attached complete a 3- to 8-membered ring containing carbon atoms and optionally containing a heteroatom selected from O, S, or NR⁵⁰ and which is optionally substituted;
R¹⁴ is independently selected from the group consisting of hydrogen, alkyl, arylalkyl, cycloalkylalkyl, heteroarylalkyl, heterocyclylalkyl and halo, wherein alkyl, arylalkyl, cycloalkylalkyl, heteroarylalkyl and heterocyclylalkyl are optionally substituted one or more times;
R¹⁶ is selected from the group consisting of cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii): 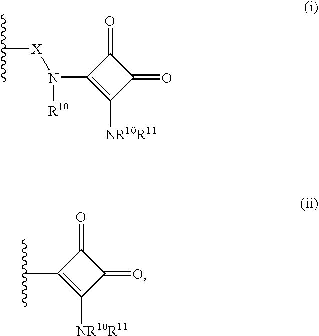
wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;
R²² and R²³ are independently selected from the group consisting of hydrogen, hydroxy, halo, alkyl, cycloalkyl, alkoxy, alkenyl, alkynyl, NO₂, NR¹⁰R¹¹, CN, SR¹⁰, SSR¹⁰, PO₃R¹⁰, NR¹⁰NR¹⁰R¹¹, NR¹⁰N═CR¹⁰R¹¹, NR¹⁰SO₂R¹¹, C(O)OR¹⁰, C(O)NR¹⁰R¹¹, SO₂R¹⁰, SO₂NR¹⁰R¹¹ and fluoroalkyl, wherein alkyl, cycloalkyl, alkoxy, alkenyl, alkynyl, and fluoroalkyl are optionally substituted one or more times;
R³⁰ is selected from the group consisting of alkyl and (C₀-C₆)-alkyl-aryl, wherein alkyl and aryl are optionally substituted;
R⁵⁰ is selected from the group consisting of hydrogen, alkyl, aryl, heteroaryl, C(O)R¹⁰, C(O)NR¹⁰R¹¹, SO₂R¹⁰ and SO₂NR¹⁰R¹¹, wherein alkyl, aryl, and heteroaryl are optionally substituted;
E is selected from the group consisting of a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and 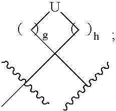
U is selected from the group consisting of C(R⁵R¹⁰), NR⁵, O, S, S═O and S(═O)₂;
W¹ is selected from the group consisting of O, NR⁵, S, S═O, S(═O)₂, N(R¹⁰)(C═O), N(R¹⁰)S(═O)₂ and S(═O)₂N(R¹⁰);
X is selected from the group consisting of a bond and (CR¹⁰R¹¹)_{w}E(CR¹⁰R¹¹)_{w};
g and h are independently selected from 0-2;
w is independently selected from 0-4;
x is selected from 0 to 2;
y is selected from 1 and 2;
with the proviso that R² and R³ are not both hydrogen; and
N-oxides, pharmaceutically acceptable salts, prodrugs, formulation, polymorphs, racemic mixtures and stereoisomers thereof.

2. A compound according to claim 1, wherein R¹ is selected from the group consisting of: 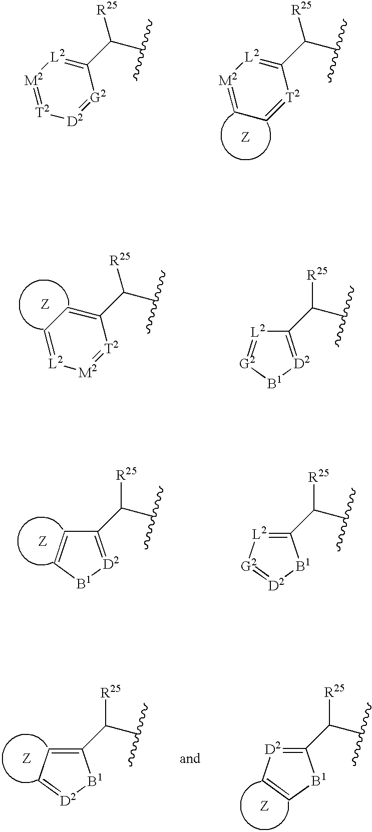
wherein:
R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂—NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, heteroaryl are optionally substituted one or more times;
R²⁵ is selected from the group consisting of hydrogen, alkyl, cycloalkyl, CO₂R¹⁰, C(O)NR¹⁰R¹¹ and haloalkyl, wherein alkyl, cycloalkyl, and haloalkyl are optionally substituted one or more times;
B₁ is selected from the group consisting of NR¹⁰, O and S(O)ₓ;
D², G², L², M² and T² are independently selected from the group consisting of CR¹⁸ and N; and
Z is a 5- to 8-membered ring selected from the group consisting of cycloalkyl, heterocycloalkyl, or a 5- to 6-membered ring selected from the group consisting of aryl and heteroaryl, wherein cycloalkyl, heterocycloalkyl, aryl and heteroaryl are optionally substituted one or more times.

3. A compound according to claim 1, wherein R¹ is selected from the group consisting of:

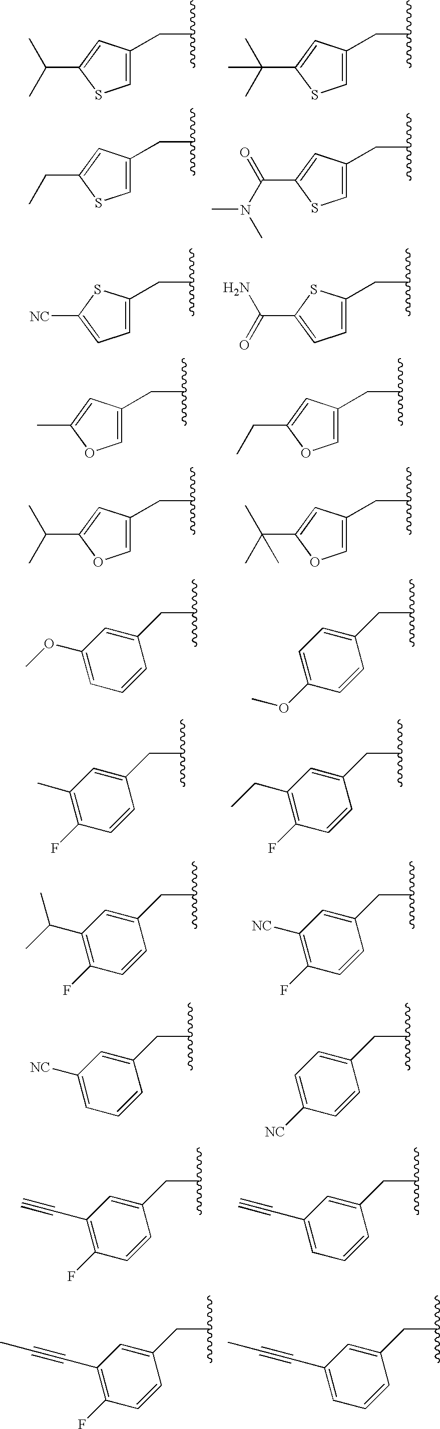

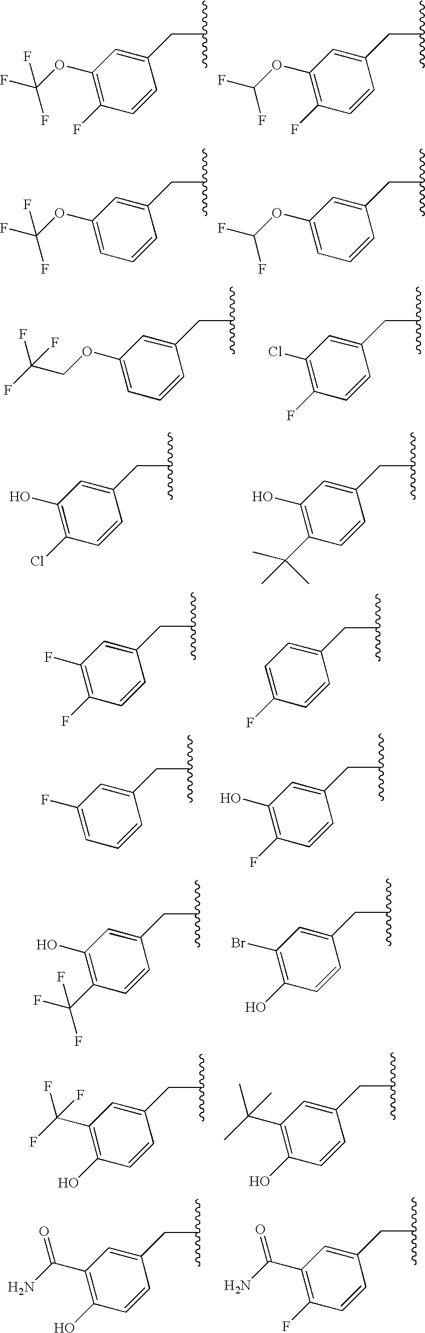

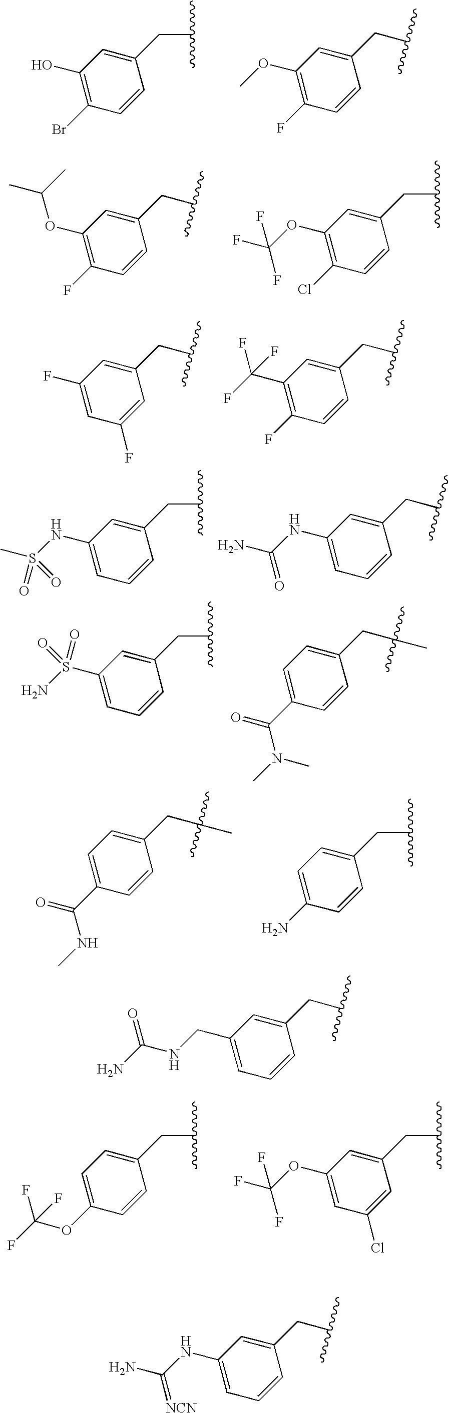

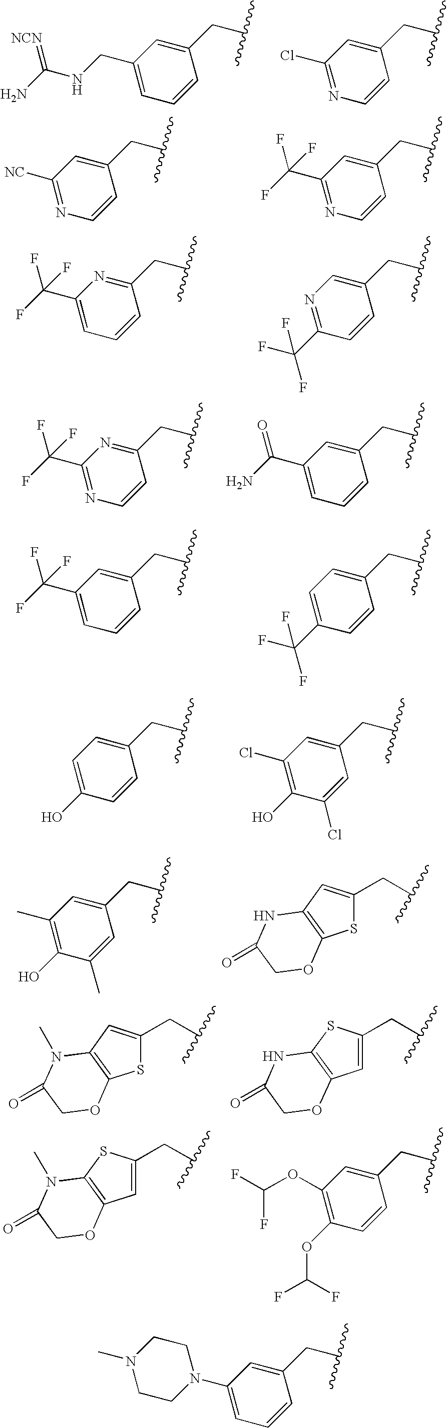

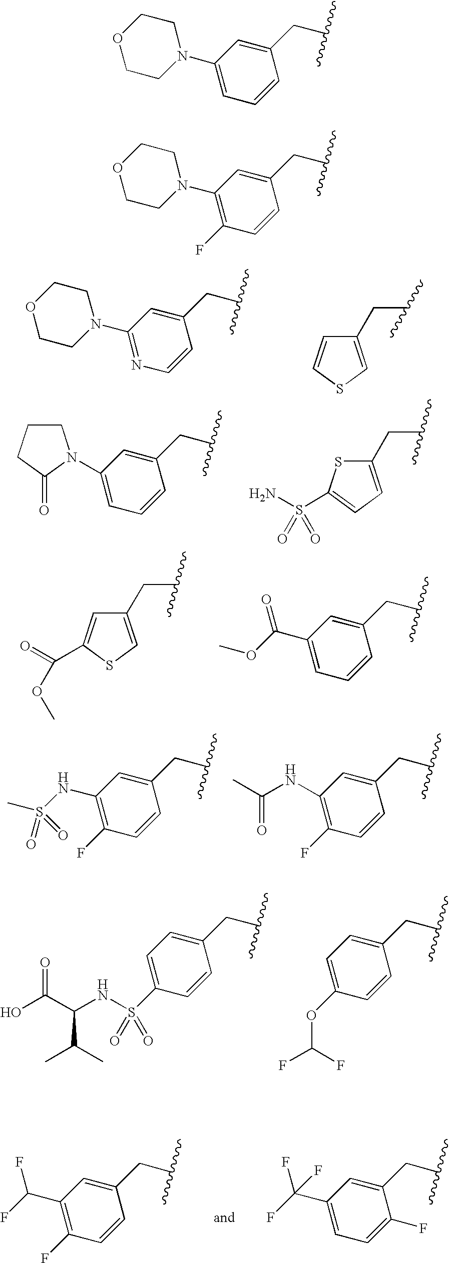

4. A compound according to claim 1, wherein R¹ is selected from the group consisting of: 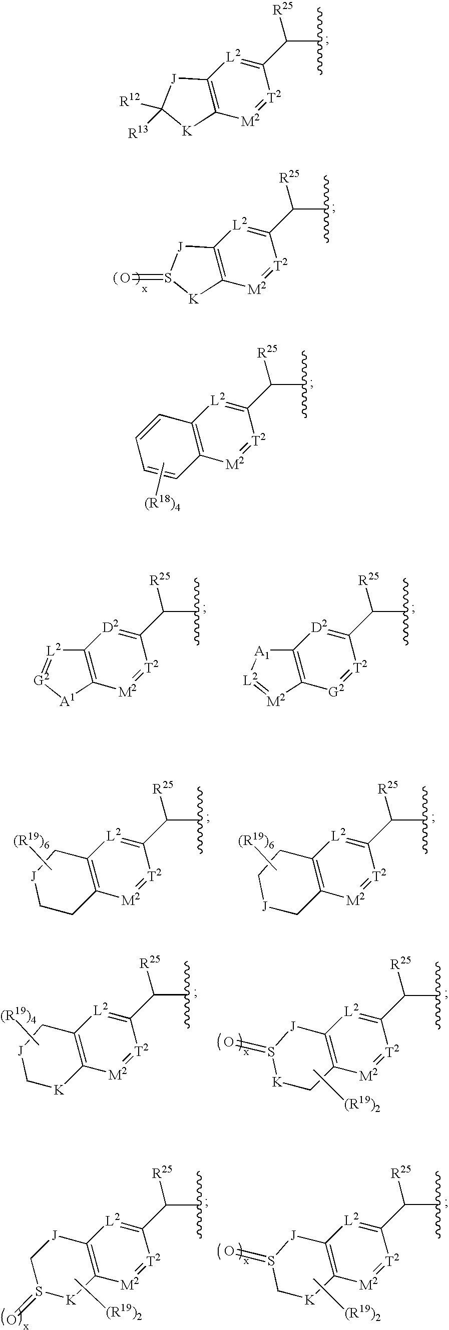 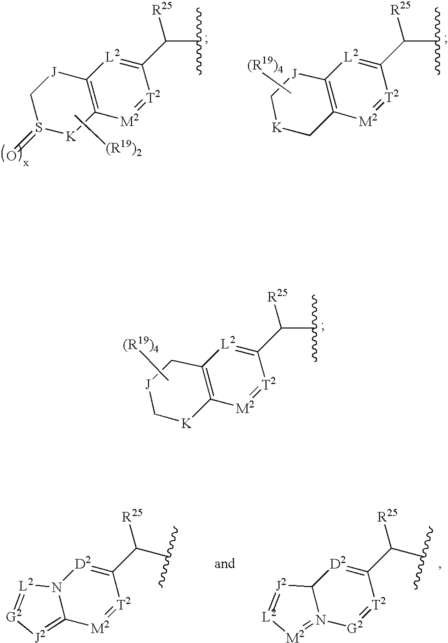
wherein:
R¹² and R¹³ are independently selected from the group consisting of hydrogen, alkyl and halo, wherein alkyl is optionally substituted one or more times, or optionally R¹² and R¹³ together form ═O, ═S or ═NR¹⁰;
R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;
R¹⁹ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;
R²⁵ is selected from the group consisting of hydrogen, alkyl, cycloalkyl, CO₂R¹⁰, C(O)NR¹⁰R¹¹ and haloalkyl, wherein alkyl, cycloalkyl, and haloalkyl are optionally substituted one or more times;
J and K are independently selected from the group consisting of CR¹⁰R¹⁸, NR¹⁰, O and S(O)ₓ;
A₁ is selected from the group consisting of NR¹⁰, O and S(O)ₓ; and
D², G², J², L², M² and T² are independently selected from the group consisting of CR¹⁸ and N.

5. A compound according to claim 1, wherein R¹ is selected from the group consisting of:

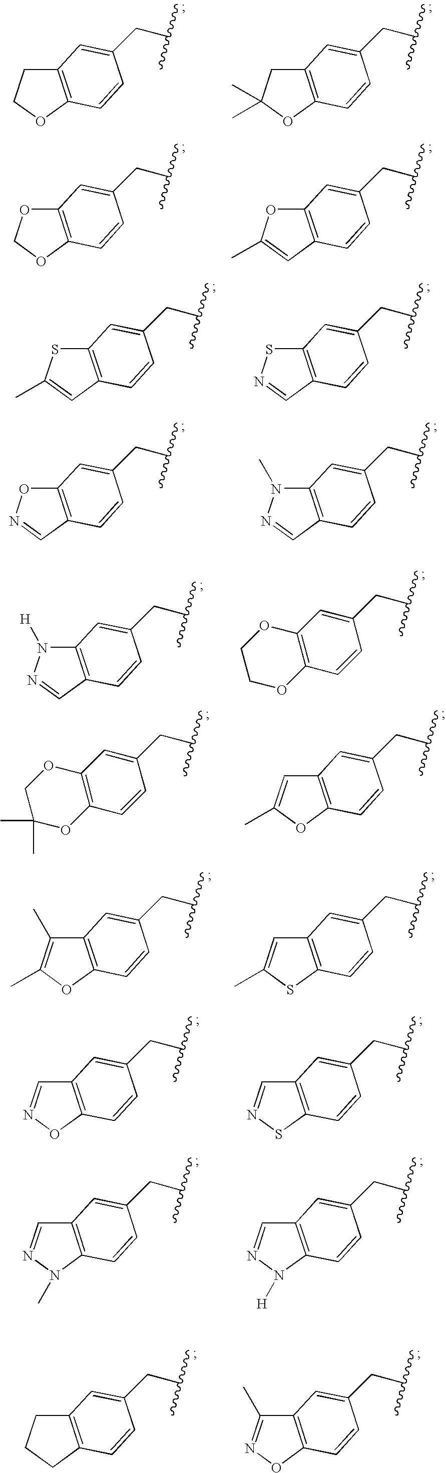

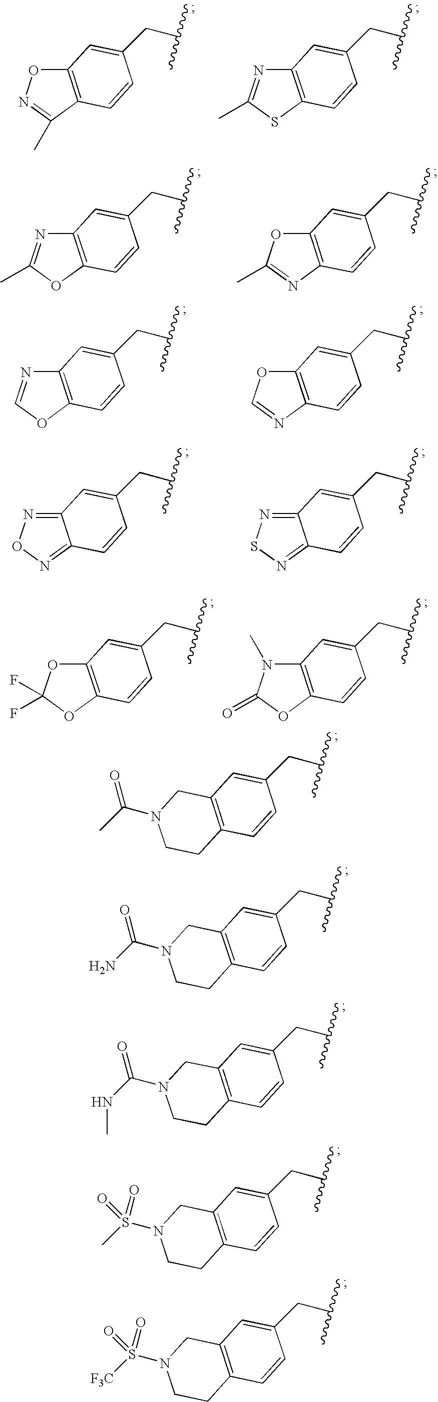

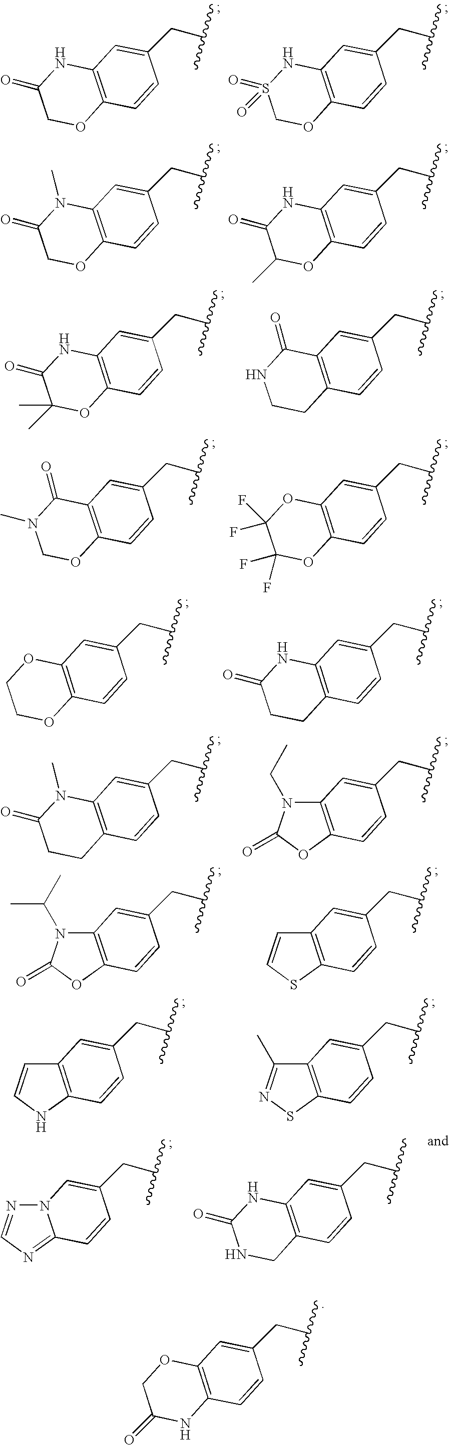

6. A compound according to claim 1, wherein R¹ is selected from the group consisting of: 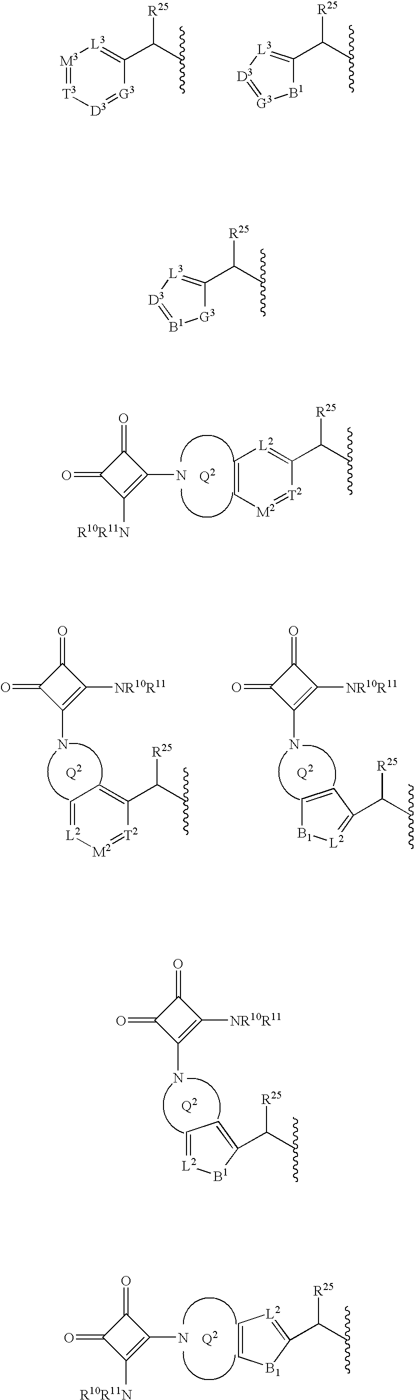 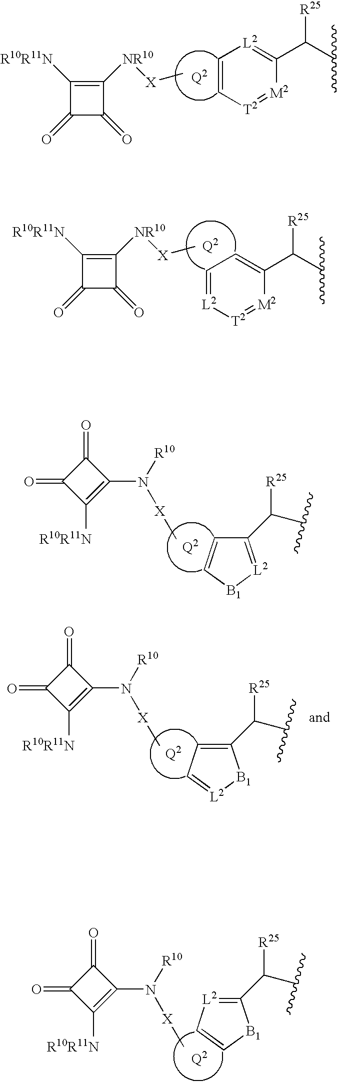
wherein
R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;
R¹⁹ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;
R²⁵ is selected from the group consisting of hydrogen, alkyl, cycloalkyl, CONR¹⁰R¹¹ and haloalkyl, wherein alkyl, cycloalkyl and haloalkyl are optionally substituted one or more times;
L², M², and T² are independently selected from the group consisting of CR¹⁸ and N;
D³, G³, L³, M³, and T³ are independently selected from N, CR¹⁸, (i), or (ii), 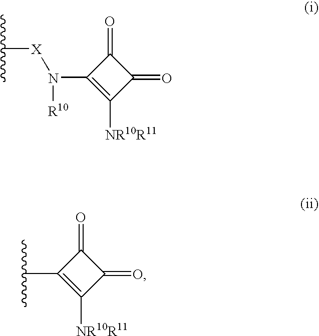
with the proviso that one of L³, M³, T³, D³, and G³ is (i) or (ii);
B₁ is selected from the group consisting of NR¹⁰, O and S(O)ₓ; and
Q² is a 5- to 8-membered ring selected from the group consisting of cycloalkyl, heterocycloalkyl, aryl, and heteroaryl, which is optionally substituted one or more times with R¹⁹.

7. A compound of claim 6, wherein R¹ is selected from the group consisting of:

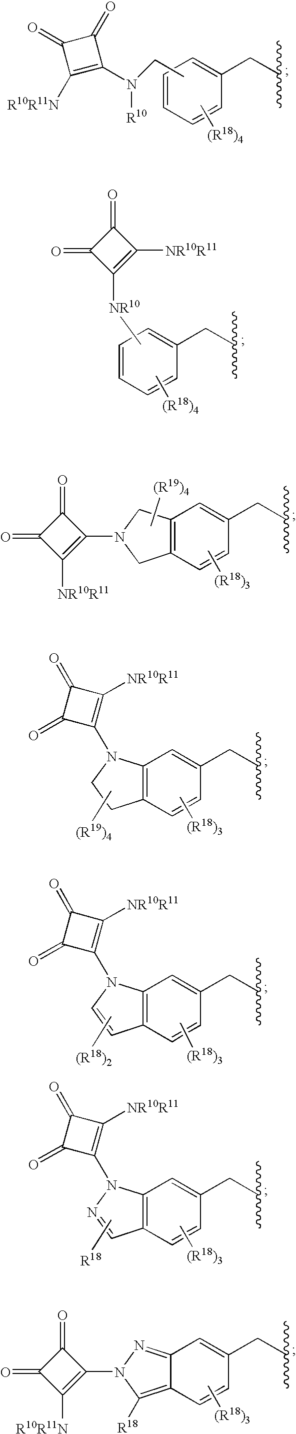

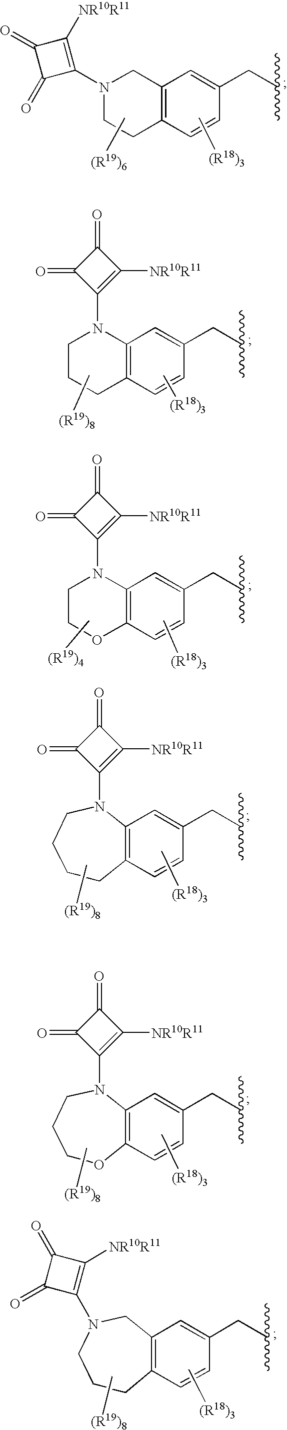

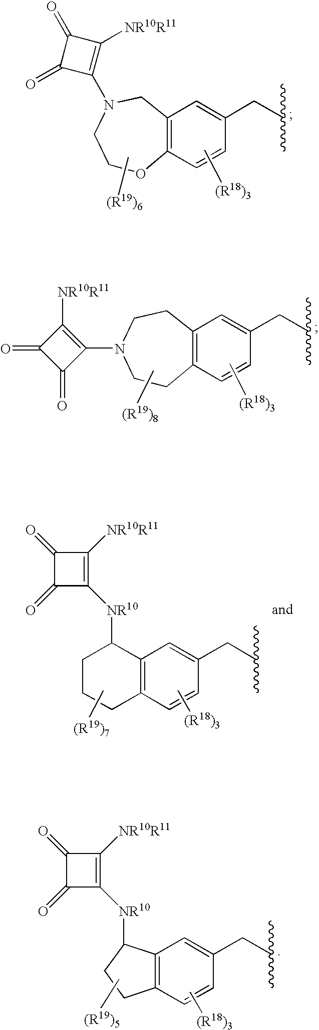

8. A compound of claim 1, wherein R¹ is selected from the group consisting of:

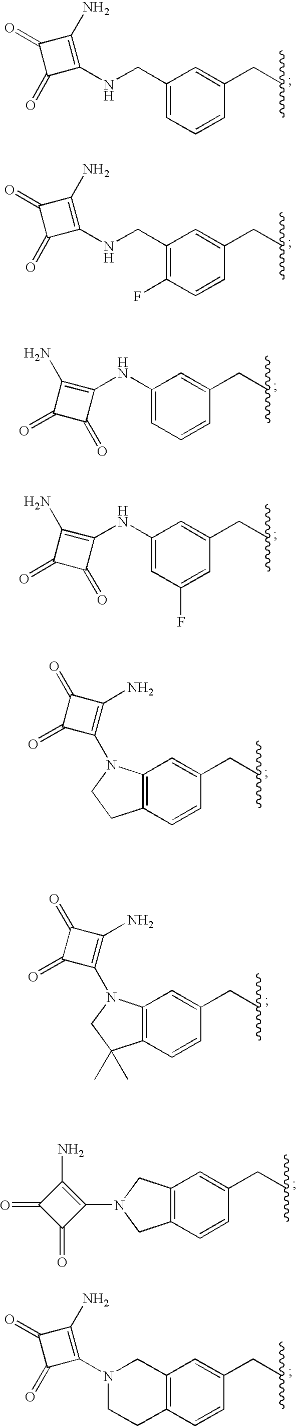

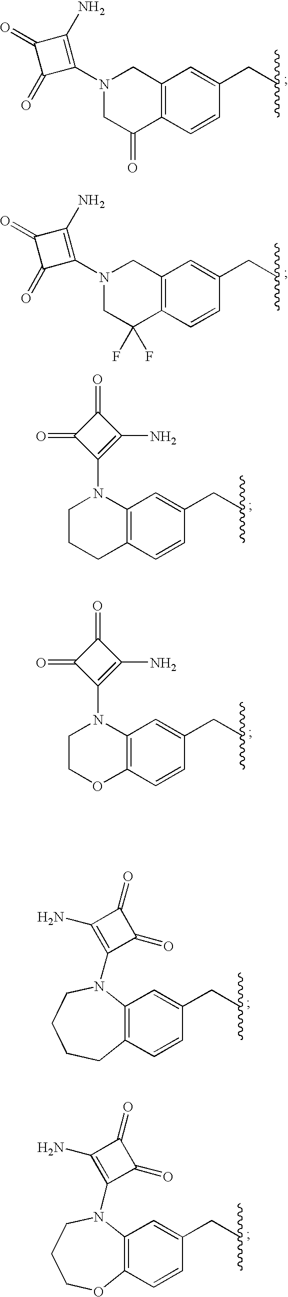

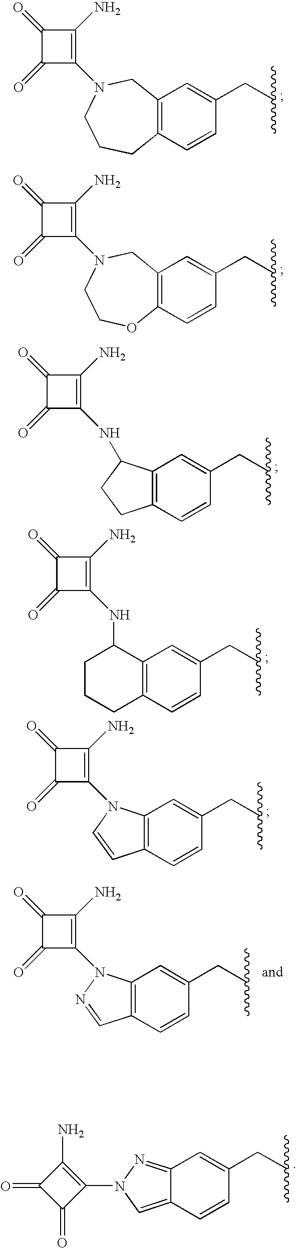

9. A compound of claim 1, wherein
R² is selected from the group consisting of alkyl, haloalkyl, fluoroalkyl, cycloalkyl, alkenyl, alkynyl, aryl, heteroaryl, cycloalkyl-alkyl, arylalkyl, heteroarylalkyl, COOR¹⁰, CONR¹⁰R¹¹, SO₂R¹⁰ and SO₂NR¹⁰R¹¹ wherein alkyl, haloalkyl, fluoroalkyl, cycloalkyl, alkenyl, alkynyl, aryl, heteroaryl, cycloalkyl-alkyl, arylalkyl, and heteroarylalkyl are optionally substituted one or more times; and
R³ is hydrogen.

10. A compound of claim 1, wherein
R² is selected from the group consisting of alkyl, haloalkyl, fluoroalkyl, COOR¹⁰, CONR¹⁰R¹¹, wherein alkyl, haloalkyl, fluoroalkyl are optionally substituted one or more times; and
R³ is hydrogen.

11. A compound of claim 1, wherein
R² is alkyl, which is optionally substituted one or more times; and
R³ is hydrogen.

12. A compound according to claim 1, wherein R⁴ is selected from the group consisting of: 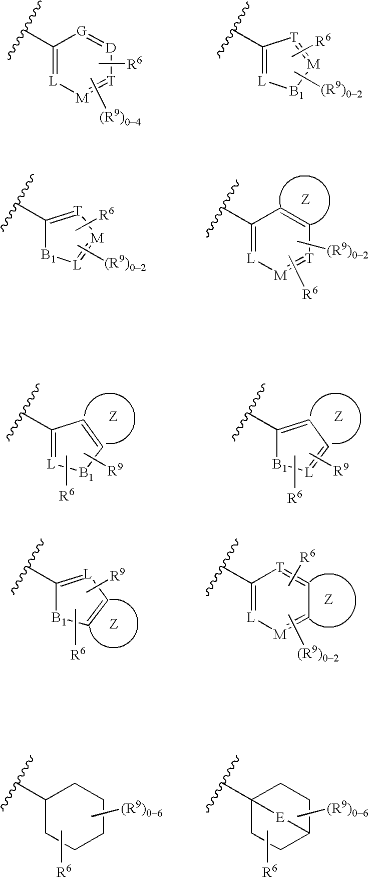
wherein
R⁶ is independently selected from the group consisting of R⁹, alkenyl, alkynyl, cycloalkyl, heterocycloalkyl, bicycloalkyl, heterobicycloalkyl, spiroalkyl, spiroheteroalkyl, aryl, heteroaryl, C(O)OR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓ R¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹¹—CN, O—(C₀-C₆)-alkyl-C(O)NR¹⁰R¹¹, S(O)ₓ—(C₀-C₆)alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl, wherein each R⁶ group is optionally substituted by one or more R¹⁴ groups;
B₁ is selected from NR¹⁰, O or S(O)ₓ
L, M, T, D and G are independently selected from C or N;
Z is a 5- to 8-membered ring selected from the group consisting of cycloalkyl, heterocycloalkyl, or a 5- to 6-membered ring selected from the group consisting of aryl and heteroaryl, wherein cycloalkyl, heterocycloalkyl, aryl and heteroaryl are optionally substituted one or more times.

13. A compound according to claim 1, wherein R⁴ is selected from the group consisting of: 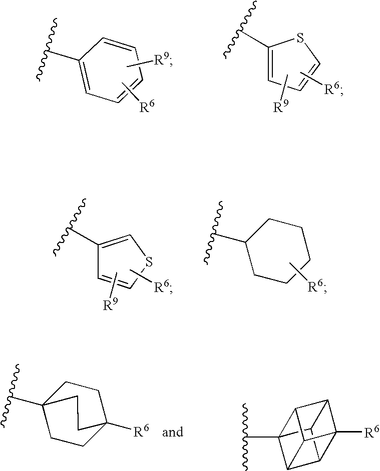
wherein
R⁶ is selected from the group consisting of 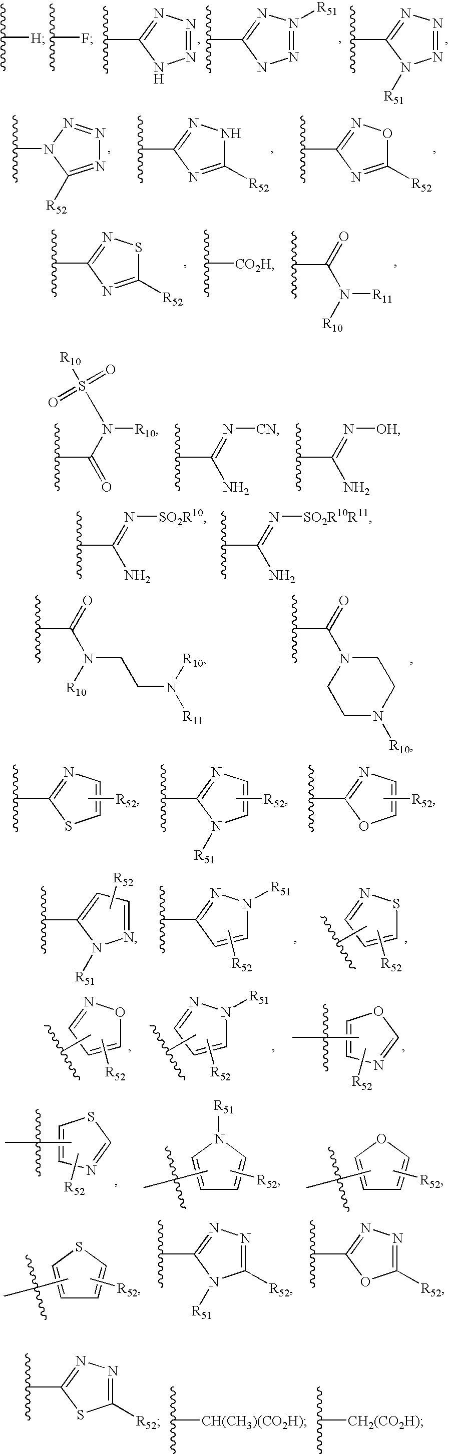 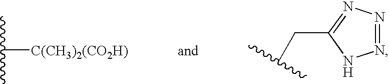
R⁹ is selected from the group consisting of hydrogen, alkyl, halo, CF₃, COR¹⁰, OR¹¹, NR¹⁰R¹¹, NO₂, CN, wherein alkyl is optionally substituted;
R⁵¹ is selected from the group consisting of hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted;
R⁵² is selected from the group consisting of hydrogen, halo, hydroxy, alkoxy, fluoroalkoxy, alkyl, aryl, heteroaryl, arylalkyl, cycloalkylalkyl, heteroarylalkyl, haloalkyl, C(O)NR¹⁰R¹¹ and SO₂NR¹⁰R¹¹, wherein alkoxy, fluoroalkoxy, alkyl, aryl, heteroaryl, arylalkyl, cycloalkylalkyl, heteroarylalkyl and haloalkyl are optionally substituted.

14. A compound according to claim 12, wherein R⁶ is COOH or heteroaryl.

15. A compound according to claim 12, wherein R⁶ is selected from the group consisting of COOH, dioxole, imidazole, furan, thiazole, isothiazole, isoxazole, morpholine, 1,2,4-oxadiazole, 1,3,4-oxadiazole, 1,2,4-oxadiazole, 1,2-oxazine, 1,3-oxazine, 1,4-oxazine, oxirane, oxazole, 5-oxo-1,2,4-oxadiazole, 5-oxo-1,2,4-thiadiazole, piperzine, piperidine, pyran, pyrazine, pyrazole, pyridazine, pyridine, pyrimidine, pyrrole, pyrrolidine, tetrazine, tetrazole, thiazine, 1,2,3-thiadiazole, 1,2,4-thiadiazole, 1,3,4-thiadiazole, 1,2,5-thiadiazole, thiatriazole, 1,2-thiazine, 1,3-thiazine, 1,4-thiazine, thiazole, 5-thioxo-1,2,4-diazole, thiomorpholine, thiophene, thiopyran, 1,2,3-triazine, 1,2,4-triazine, 1,3,5-triazine, 1,2,4-triazole, 1,2,3-triazole, and triazolones, wherein R⁶ is optionally substituted.

16. A compound according to claim 1, wherein R⁴ is selected from the group consisting of:

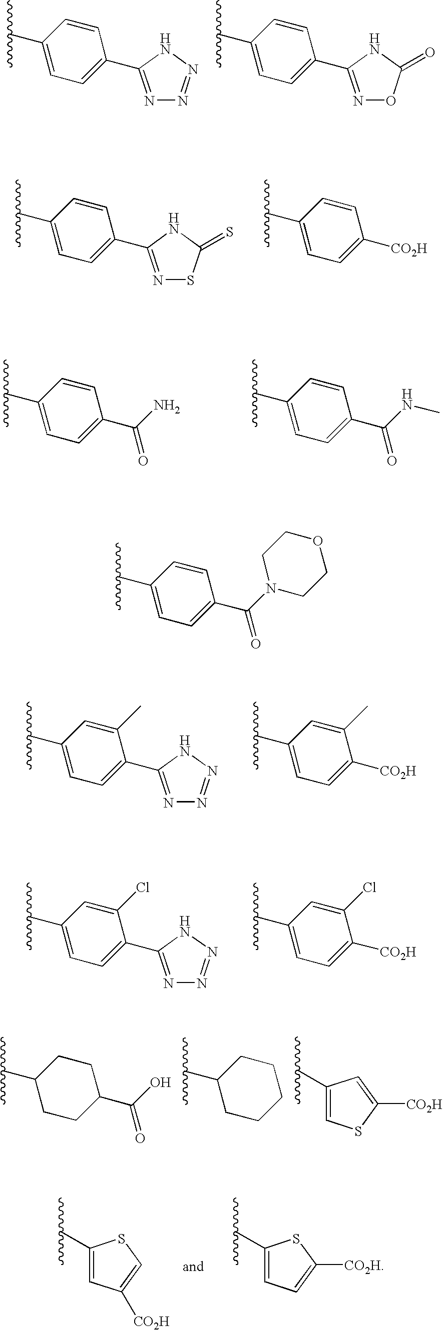

17. A compound according to claim 1, selected from the group consisting of:

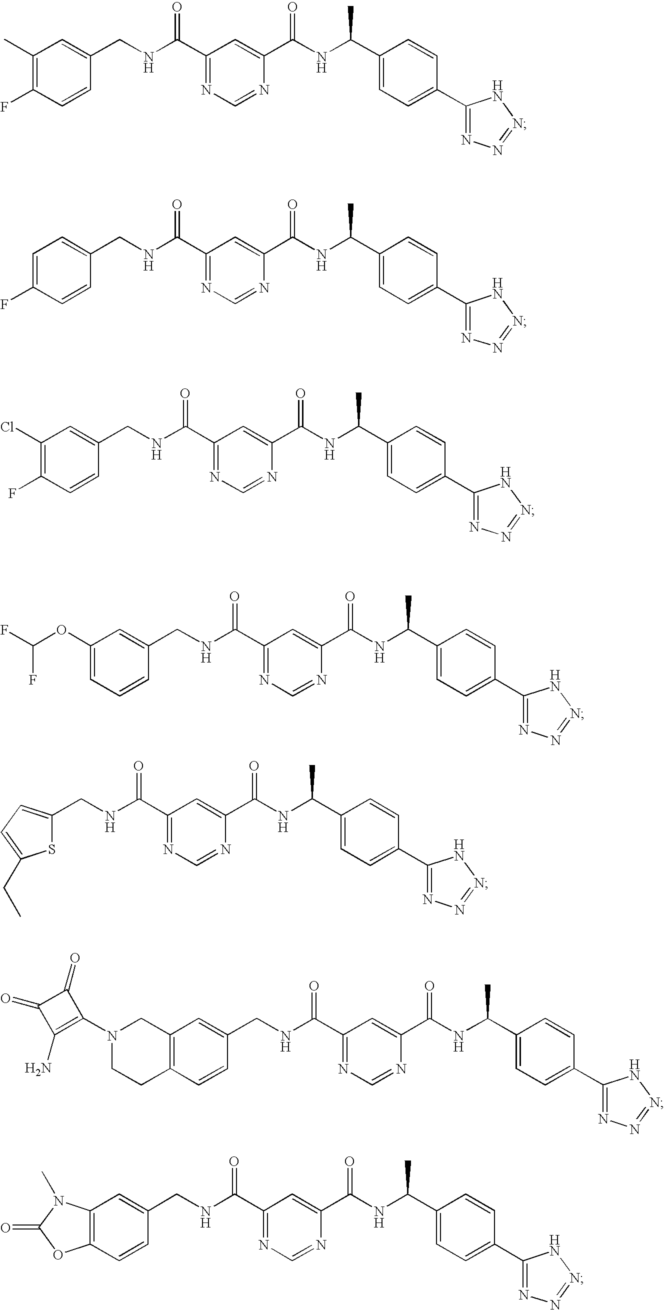

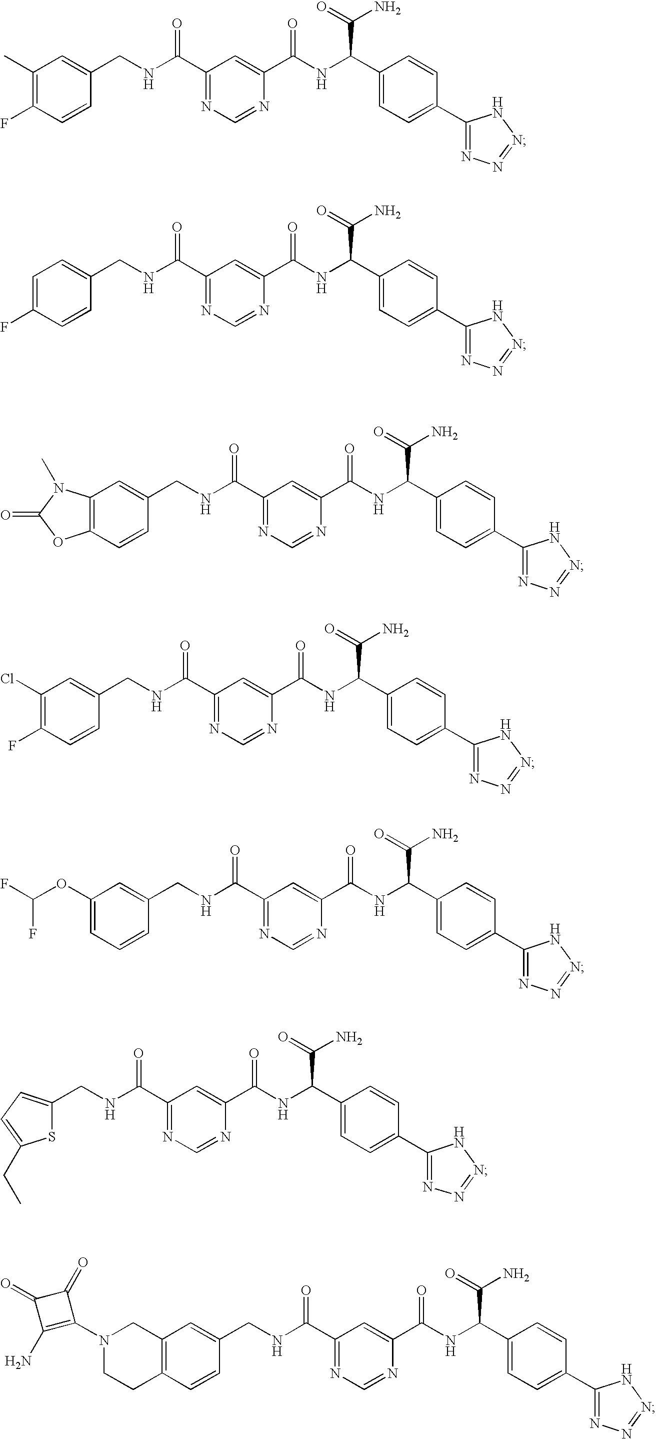

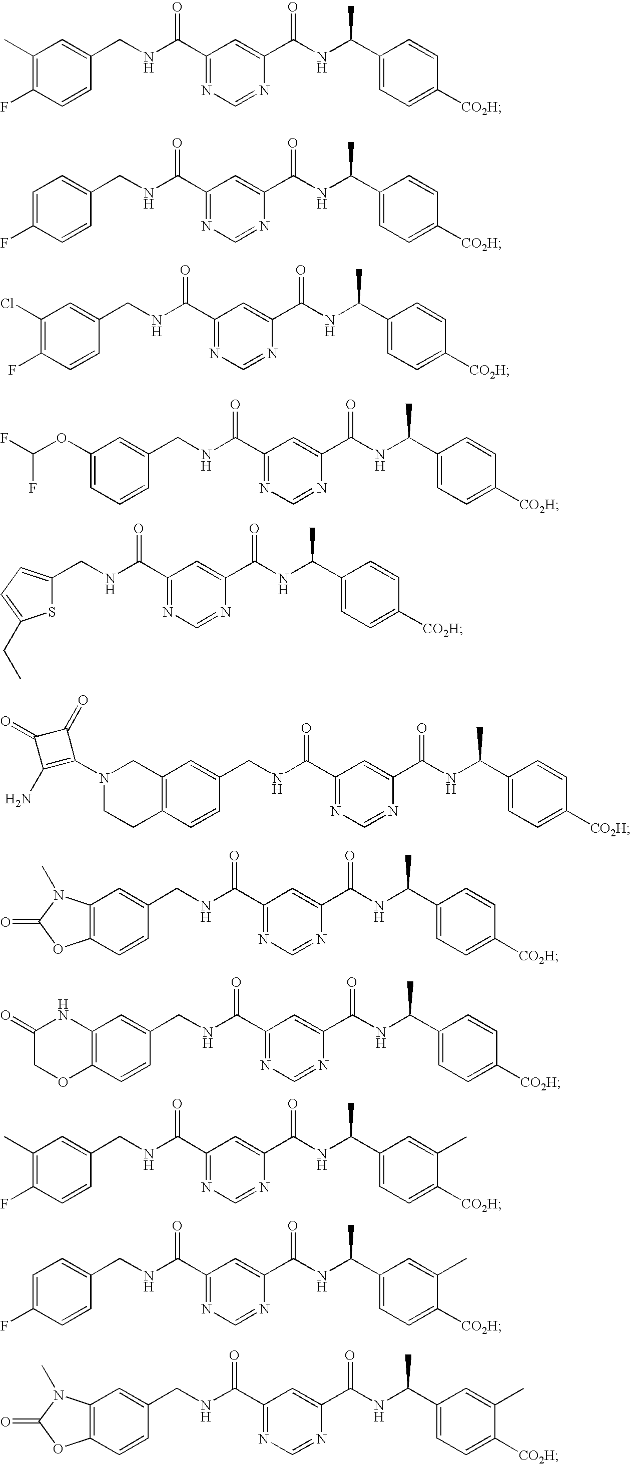

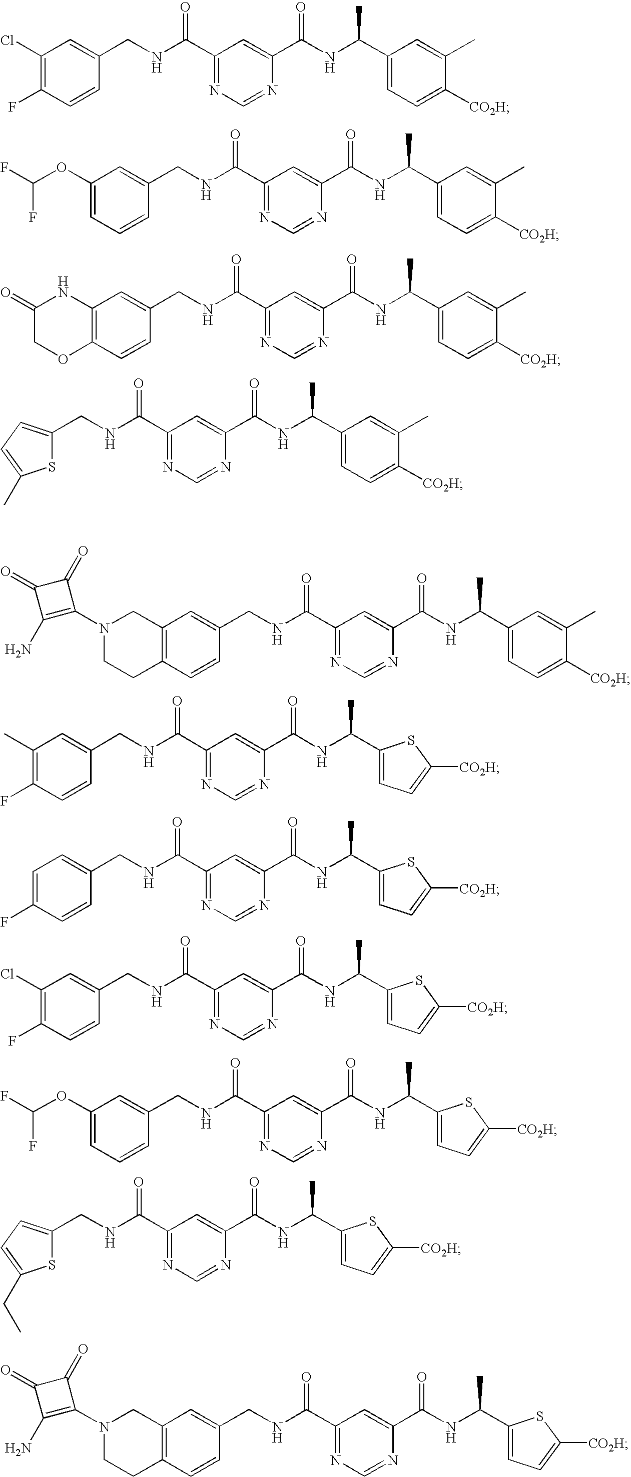

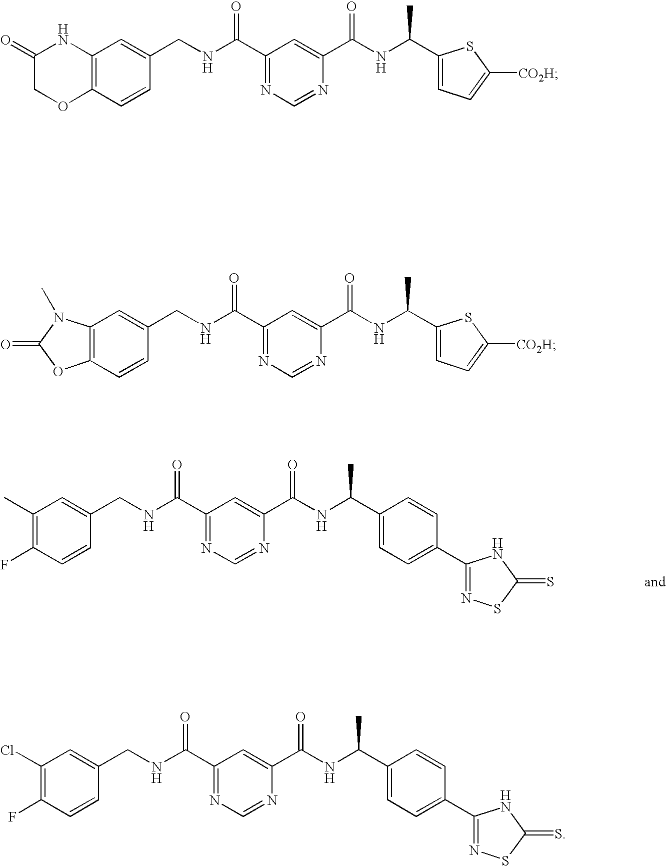

18. A compound according to claim 1, which comprises: 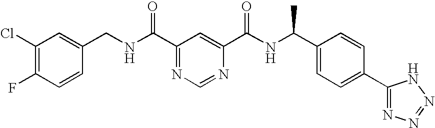
or a pharmaceutically acceptable salt thereof.

19. A compound according to claim 1, which comprises: 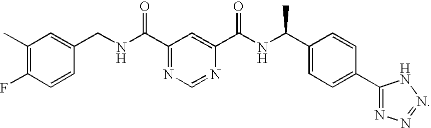
or a pharmaceutically acceptable salt thereof.

20. A compound according to claim 1, which comprises: 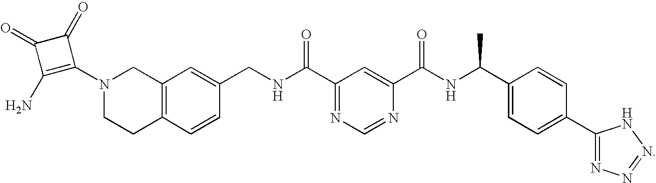
or a pharmaceutically acceptable salt thereof.

21. A compound according to claim 1, which comprises: 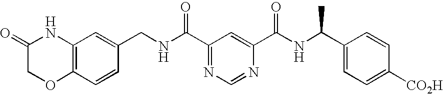
or a pharmaceutically acceptable salt thereof.

22. A compound according to claim 1, which comprises: 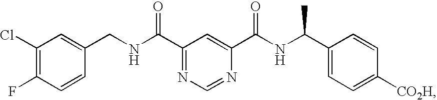
or a pharmaceutically acceptable salt thereof.

23. A compound according to claim 1, which comprises: 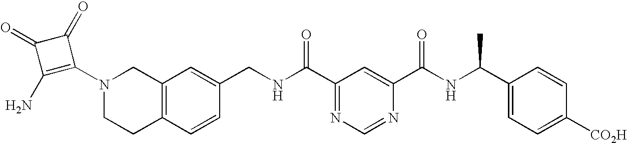
or a pharmaceutically acceptable salt thereof.

24. A compound according to claim 1, which comprises: 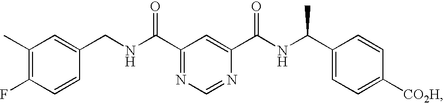
or a pharmaceutically acceptable salt thereof.

25. A compound according to claim 1, which comprises: 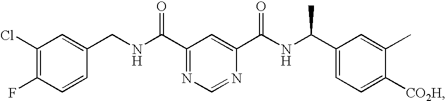
or a pharmaceutically acceptable salt thereof.

26. A compound according to claim 1, which comprises: 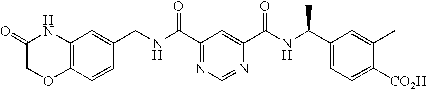
or a pharmaceutically acceptable salt thereof.

27. A compound according to claim 1, which comprises: 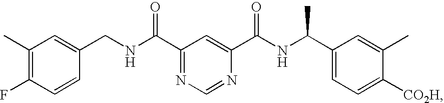
or a pharmaceutically acceptable salt thereof.

28. A compound according to claim 1, which comprises: 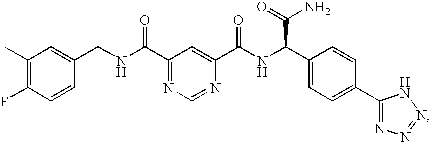
or a pharmaceutically acceptable salt thereof.

29. A compound according to claim 1, which comprises: 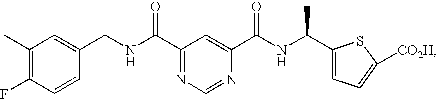
or a pharmaceutically acceptable salt thereof.

30. A compound according to claim 1, which comprises: 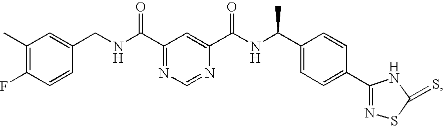
or a pharmaceutically acceptable salt thereof.

31. A pharmaceutical composition comprising an effective amount of a compound according to claim 1 and a pharmaceutically acceptable carrier.

32. A method of inhibiting a metalloprotease enzyme, comprising administering a compound selected from claim 1.

33. The method of claim 32, wherein said metalloprotease enzyme is selected from the MMP-13 enzyme.

34. A method of treating a metalloprotease mediated disease, comprising administering to a subject in need of such treatment an effective amount of a compound of claim 1.

35. The method of claim 34, wherein said metalloprotease mediated disease is a MMP-13 mediated disease.

36. The method according to claim 34, wherein the disease is selected from rheumatoid arthritis, osteoarthritis, abdominal aortic aneurysm, cancer, inflammation disorders, artherosclerosis, pain, inflammatory pain, bone pain, joint pain, chronic obstructive pulmonary disease, and multiple sclerosis.

37. Use of a compound selected from claim 1 in the manufacture of a medicament for the treatment of a disease mediated by a metalloprotease enzyme.

38. Use of a compound of claim 37, wherein said metalloprotease enzyme is selected from the MMP-13 enzyme.

39. Use of a compound according to claim 1, wherein a drug, agent or therapeutic is used in combination with said compound of claim 1, said drug, agent or therapeutic being selected from the group consisting of: (a) a disease modifying antirheumatic drug; (b) a nonsteroidal anti-inflammatory drug; (c) a COX-2 selective inhibitor; (d) a COX-1 inhibitor; (e) an immunosuppressive; (f) a steroid; (g) a biological response modifier; and (h) other anti-inflammatory agents or therapeutics useful for the treatment of chemokine mediated diseases.

40. The use of claim 39 wherein said disease modifying antirheumatic drug is selected from the group consisting of methotrexate, azathioptrineluflunomide, penicillamine, gold salts, mycophenolate, mofetil and cyclophosphamide.

41. The use of claim 39 wherein said nonsteroidal anitinflammatory drug is selected from the group consisting of piroxicam, ketoprofen, naproxen, indomethacin, and ibuprofen.

42. The use of claim 39 wherein said COX-2 selective inhibitor is selected from the group consisting of rofecoxib, celecoxib, and valdecoxib.

43. The use of claim 39 wherein said COX-1 inhibitor is piroxicam.

44. The use of claim 39 wherein said immunosuppressive is selected from the group consisting of methotrexate, cyclosporin, leflunimide, tacrolimus, rapamycin and sulfasalazine.

45. The use of claim 39 wherein said steroid is selected from the group consisting of p-methasone, prednisone, cortisone, prednisolone and dexamethasone.

46. The use of claim 39 wherein said biological response modifier is selected from the group consisting of anti-TNF antibodies, TNF-α antagonists, IL-1 antagonists, anti-CD40, anti-CD28, IL-10 and anti-adhesion molecules.

47. The use of claim 39 wherein said other anti-inflammatory agents or therapeutics are selected from the group consisting of p38 kinase inhibitors, PDE4 inhibitors, TACE inhibitors, chemokine receptor antagonists, thalidomide, leukotriene inhibitors and other small molecule inhibitors of pro-inflammatory cytokine production.

48. A pharmaceutical composition comprising:
a) an effective amount of a compound according to claim 1;
b) a pharmaceutically acceptable carrier; and
c) a member selected from the group consisting of: (a) a disease modifying antirheumatic drug; (b) a nonsteroidal anti-inflammatory drug; (c) a COX-2 selective inhibitor; (d) a COX-1 inhibitor; (e) an immunosuppressive; (f) a steroid; (g) a biological response modifier; and (h) a small molecule inhibitor of pro-inflammatory cytokine production.

49. The pharmaceutical composition according to claim 48, wherein said COX-2 selective inhibitor is selected from the group consisting of rofecoxib, celecoxib, and valdecoxib.

50. The pharmaceutical composition according to claim 48, wherein said COX-1 inhibitor is piroxicam.

51. Use of a compound selected from the group consisting of: 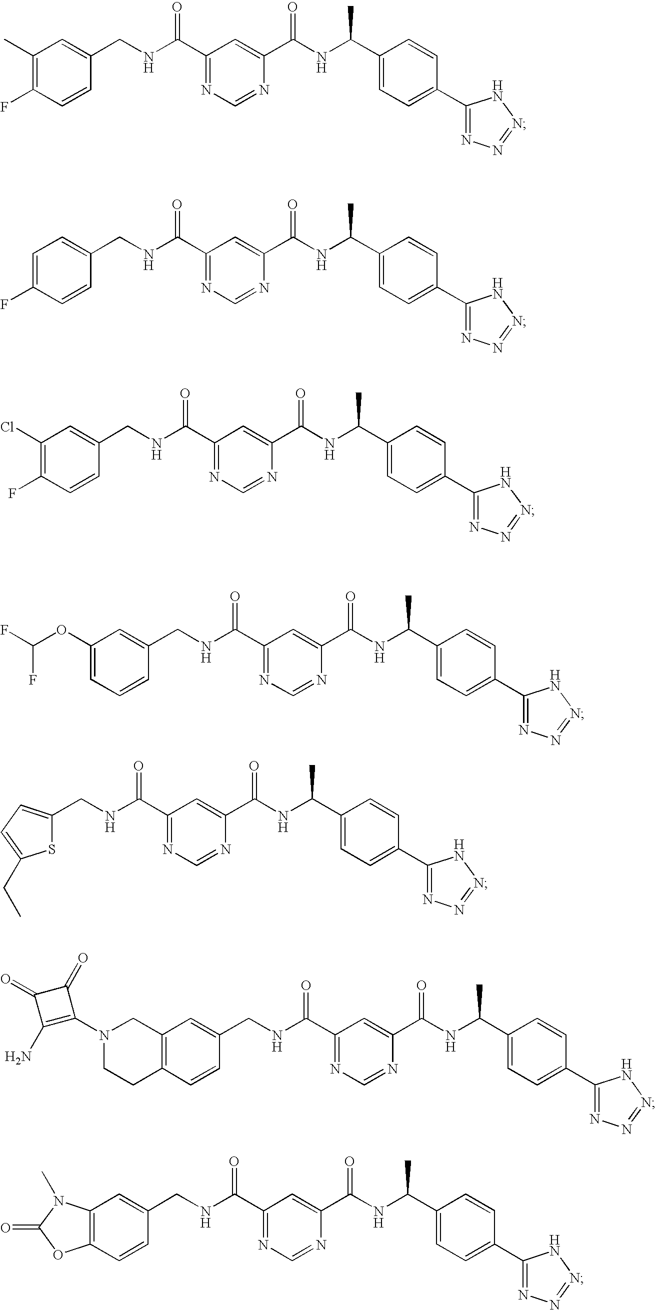 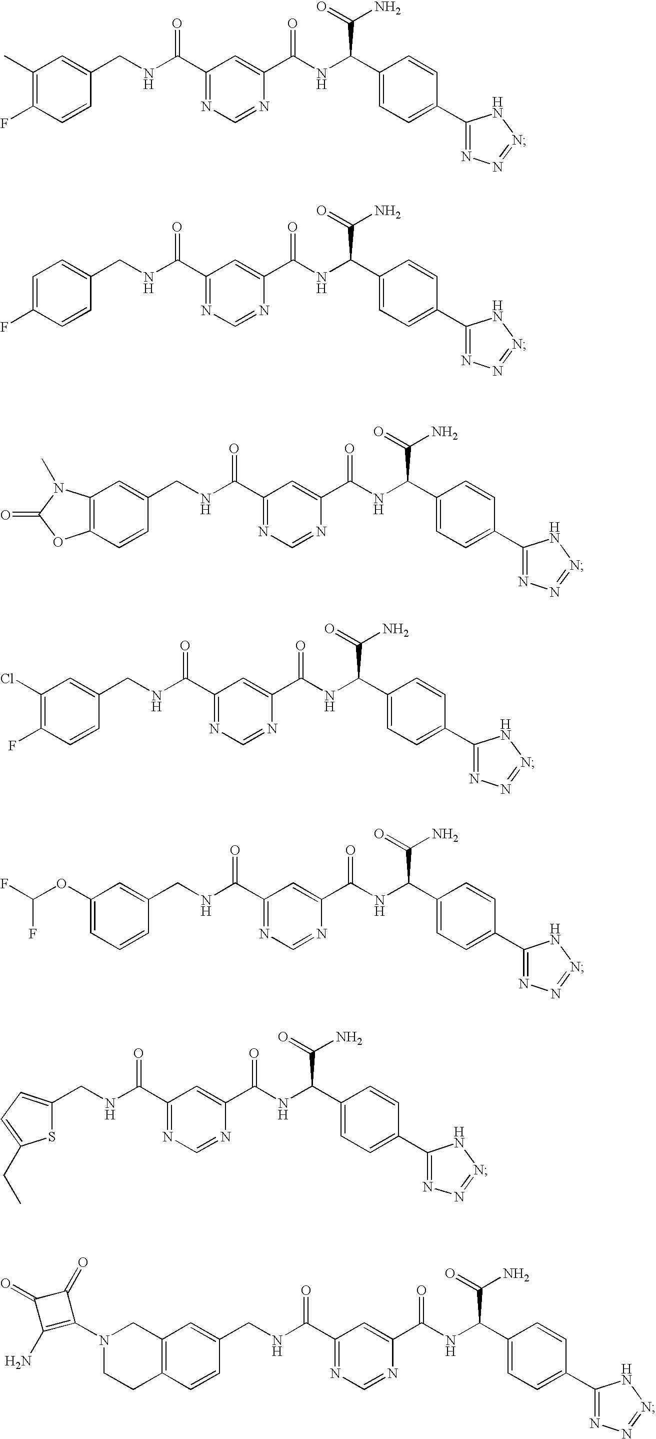 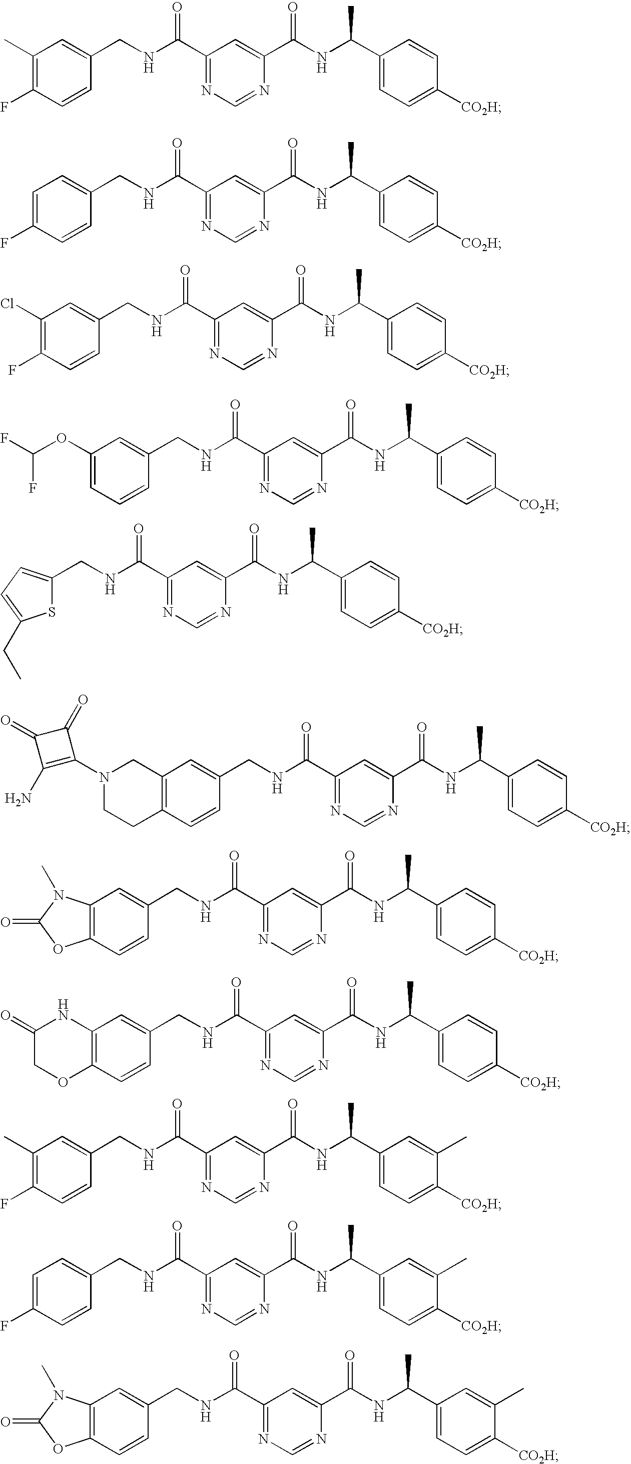 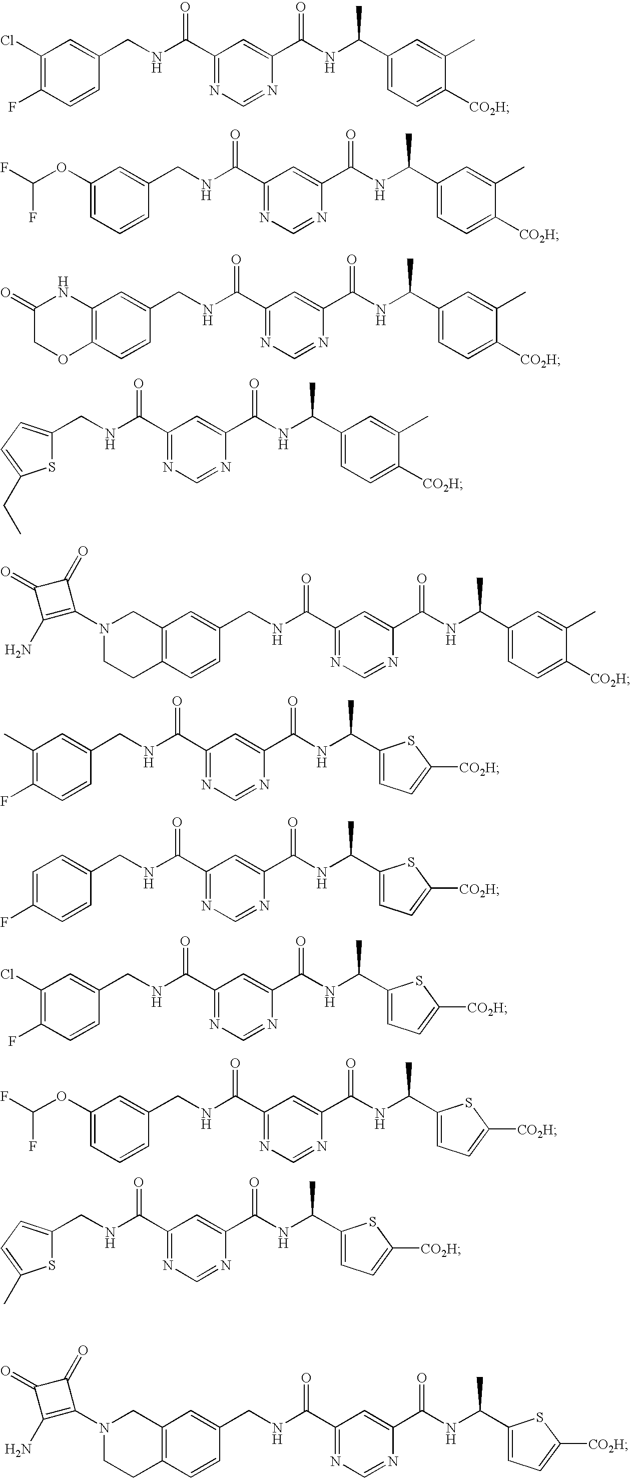 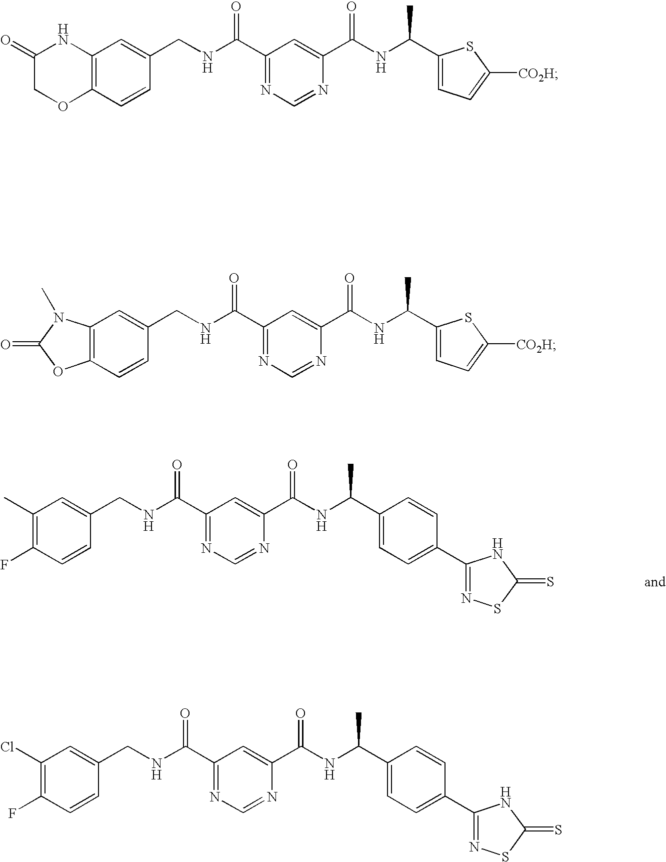
or a pharmaceutically acceptable salt thereof, in the manufacture of a medicament for the treatment of a disease mediated by an MMP-13 enzyme.

52. The use of a compound according to claim 51, wherein a drug, agent or therapeutic is used in combination with said compound, said drug, agent or therapeutic being selected from the group consisting of: (a) a disease modifying antirheumatic drug; (b) a nonsteroidal anti-inflammatory drug; (c) a COX-2 selective inhibitor; (d) a COX-1 inihibitor; (e) an immunosuppressive; (f) a steroid; (g) a biological response modifier; and (h) other anti-inflammatory agents or therapeutics useful for the treatment of chemokine mediated diseases.
